(19) United States
(12) Patent Application Publication
(10) Pub. No.: US 2015/0297745 A1
(43) Pub. Date: Oct. 22, 2015

(54) Agents and Methods

(71) Applicant: THE UNIVERSITY OF BIRMINGHAM, Birmingham (GB)

(72) Inventors: Mark Cobbold, Birmingham (GB); David Millar, Birmingham (GB)

(73) Assignee: University of Birmingham, Birmingham (GB)

(21) Appl. No.: 14/428,449

(22) PCT Filed: Sep. 17, 2013
(86) PCT No.: PCT/GB2013/052427

§ 371 Date: Mar. 16, 2015

(30) Foreign Application Priority Data
Sep. 18, 2012 (GB) 1216649.2

(51) Int. Cl. A61K 47/48; A61K 39/395; A61K 45/06; C12N 7/00; C07K 16/30; A61K 39/245

(52) U.S. Cl. CPC A61K 47/48569; C07K 16/30; A61K 47/48415; A61K 39/245; A61K 45/06; C12N 7/00; A61K 39/39558; A61K 2039/572

(57) ABSTRACT

The invention provides an agent comprising: (i) a T cell antigen, and (ii) a binding partner for any of CD22, CD23, CD30, CD74, CD70, CD43, CD44, CD47, CD54, CD58, CD62L, CD95, HLA-DR, CD59, CD55, wherein, following binding of the agent to a cell that expresses any of CD22, CD23, CD30, CD74, CD70, CD43, CD44, CD47, CD54, CD58, CD62L, CD95, HLA-DR, CD59, CD55, the agent is internalised and the T cell antigen is presented on the surface of the cell in a form that can be recognised by a T cell.

The present invention relates to immunotherapeutic agents. In particular, it relates to agents that can be used to prevent or treat a condition characterised by the presence of unwanted cells, such as tumours or other disease causing cells.

Immunotherapeutic strategies for targeting malignant disease are an active area of translational clinical research, and have been for several decades. The current models dictate that cancer represents either a functional or constitutional immunodeficiency which can be treated with immunotherapeutic manipulation of the host. These efforts can be broadly classified into two groups. The first serves to augment or support endogenous anti-tumour immunity through measures such as vaccination, cytokine support (IL-2, IFNγ) or reducing immunosuppressant environment (ipilimumab) whilst the second seeks to restore an absolute deficiency with components of a functional immune response (passive immunotherapy with antibodies, TCR transfer, Stem Cell Transplantation and adoptive immunotherapy). These approaches are unified by the argument that a highly effective functional anti-tumour immune response is indeed possible. Although irrefutable evidence exists for an effective anti-tumour immune response in some cases, this central pillar of tumour immunology is overwhelmingly countered by the current clinical reality that despite great efforts, no effective immunotherapeutics are available for the majority of patients with cancer. Almost all cancer vaccination trials have provided negative results, with those providing positive data most frequently demonstrating a small effect. The reality is that therapeutic antibodies, with a few exceptions, offer very modest clinical benefit in the area of oncology.

If a therapeutic strategy could be developed which can efficiently molecularly re-direct an endogenous anti-viral immune response to instead target malignant tissue, this may afford a new powerful and safe approach to treat malignant disease.

The majority of cytotoxic therapeutic antibodies rely on immunological effector mechanisms to deliver their anti-cancer effect such as complement dependent cytotoxicity (CDC) and Antibody Dependent Cellular Cytotoxicity (ADCC). Importantly, all cells (both healthy and malignant) have numerous mechanisms to limit attack by the immune response to avert autoimmunity. This is evident in the context of autoimmune disease where high levels of tissue-reactive antibodies, which although frequently evoke organ inflammation, rarely induce complete organ destruction. Indeed, autoimmune diseases where complete tissue destruction is observed, such as diabetes mellitus, are known to be dependent on CTL responses rather than antibody-directed mechanisms.

To improve upon the poor efficacy of therapeutic antibodies, immunoconjugates (radionuclides/toxins) and engineered antibodies which better engage with the cytotoxic effector mechanisms (e.g. glycoengineering) have been used. However clinical trials of such agents remain largely disappointing and are plagued by toxicity. One example is antibody-drug conjugates (ADCs) that have been developed to selectively target anti-tumour agents to tumours (see U.S. Pat. No. 5,773,001; U.S. Pat. No. 5,767,285; U.S. Pat. No. 5,739,116; U.S. Pat. No. 5,693,762; U.S. Pat. No. 5,585,089; US 2006/0088522; US 2011/0008840; U.S. Pat. No. 7,659,241; Hughes (2010) Nat Drug Discov 9: 665, Lash (2010); In vivo: The Business & Medicine Report 32-38; Mahato et al (2011) Adv Drug Deliv Rev 63: 659; Jeffrey et al (2006) BMCL 16: 358; Drugs R D 11(1): 85-95). ADCs generally comprise a monoclonal antibody against a target present on a tumour cell, a cytotoxic drug, and a linker that attaches the antibody to the drug. However, only a few ADCs are currently in the late stage of clinical development, and of those that are, clinical success has proven elusive.

Thus, there remains a demand for more effective immunotherapeutic agents with greater efficacy and lower toxicity.

The agents of the invention are an example of re-directed immunotherapy. This refers to the concept of re-directing an existing immune response that normally target cells harbouring foreign antigens, to target unwanted cells in conditions such as cancer. The concept requires the presentation of marker antigens on unwanted cells such that they become a target for immune cells.

WO 95/17212 describes conjugates consisting of peptidic T cell antigens and cell binding partners and their use in re-directed immunotherapy. The conjugates are said to be internalised into target cells following binding of the binding partner to surface receptors, and the T cell antigen is processed from the conjugate and expressed on the cell surface in the form of a complex with MHC molecules. Recognition of the complex by a T cell receptor induces a cytotoxic T cell response against the target cells. However, which binding partners enable internalisation and hence subsequent presentation of the T cell antigen, and which do not, cannot be predicted from WO 95/17212. The only receptors shown to present peptide antigens effectively were antigen receptors of B cells, whose normal role is to bind to and internalise antigen for presentation to helper T cells. However, WO 95/17212 offers no guidance on which other receptors, if any, are guaranteed to provide the same result.

Surprisingly and unexpectedly, the inventors have now identified particular antigens that have utility in re-directed immunotherapy, namely CD70, CD74, CD22, HLA-DR, CD23, CD30, CD43, CD44, CD47, CD54, CD55, CD58, CD59, CD62L or CD95. Targeting each of these antigens with viral derived peptides led to increased activation of virus-specific T cells in vitro, indicating that, surprisingly, each of the antigens enables internalisation of the peptides and subsequent presentation on the cell surface.

CD70 (TNFSF7) is a member of the tumour necrosis factor (TNF) superfamily. It is a type II integral membrane protein and a ligand for CD27. The protein is transiently expressed in antigen-activated T and B lymphocytes and its interaction with CD27 regulates T- and B-cell functions. In particular, the protein acts to control death, survival and co-stimulation of target cells. Although CD70 is expressed by limited subsets of normal lymphocytes and dendritic cells, it is aberrantly expressed by a broad range of hematologic malignancies and some solid tumours.

CD74 is an MHC class II chaperone and functions as a membrane receptor for the pro-inflammatory cytokine macrophage migration inhibitory factor (MIF) on immune cells. MIF binding to CD74 activates downstream signalling through the MAPK and Akt pathways and promotes cell proliferation and survival. Beside expression by immune cells, CD74 overexpression has been observed in several non-CNS cancers, and CD74 expression in these tumours is generally associated with aggressive behaviour and poor patient prognosis.

CD22 is a molecule belonging to the SIGLEC family of lectins which specifically binds sialic acid with an immunoglobulin (Ig) domain located at its N-terminus. It is found on the surface of mature B cells and to a lesser extent on some immature B cells. It is thought that CD22 is a regulatory molecule that prevents the overactivation of the immune system and the development of autoimmune diseases. It is present on many B cell malignancies including chronic lymphocytic leukaemia and non-Hodgkin's lymphoma.

HLA-DR MHC class II cell surface receptor encoded by the human leukocyte antigen complex on chromosome 6. The complex of HLA-DR and its ligand, a peptide of 9 amino acids in length or longer, constitutes a ligand for the T-cell receptor (TCR). HLA-DR molecules are upregulated in response to signalling. HLA-DR is found on various cancers including colorectal carcinoma where a increased HLA-DR expression relates to better prognostic outcome.

CD23 is the “low-affinity” receptor for IgE, an antibody isotype involved in allergy and resistance to parasites, and is important in regulation of IgE levels. Unlike many of the antibody receptors, CD23 is a C-type lectin. It is found on mature B cells, activated macrophages, eosinophils, follicular dendritic cells, and platelets. CD23 is found on B cells in B cell malignancies such as Hodgkin Lymphoma, Non-Hodgkin Lymphoma and B-cell chronic lymphocytic leukaemia.

CD30 is a cell membrane protein of the tumor necrosis factor receptor family and tumor marker. This receptor is expressed by activated, but not by resting, T and B cells. TRAF2 and TRAF5 can interact with this receptor, and mediate the signal transduction that leads to the activation of NF-kappaB. It is a positive regulator of apoptosis, and also has been shown to limit the proliferative potential of auto-reactive CD8 effector T cells and protect the body against autoimmunity. CD30 is found on T cell lymphomas including anaplastic large cell lymphoma as well as being expressed by B cell lymphomas including Hodgkin lymphoma.

CD43 is a major sialoglycoprotein on the surface of human T lymphocytes, monocytes, granulocytes, and some B lymphocytes, which appears to be important for immune function and may be part of a physiologic ligand-receptor complex involved in T-cell activation. CD43 is present in over 90% of T-cell lymphomas and may also be useful as part of a panel to demonstrate B-cell lymphoblastic lymphoma, since the malignant cells in this condition are often CD43 positive. It also stains granulocytes and their precursors, and therefore may be an effective marker for myeloid tumours,

CD44 is a cell-surface glycoprotein involved in cell-cell interactions, cell adhesion and migration. It is a receptor for hyaluronic acid and can also interact with other ligands, such as osteopontin, collagens, and matrix metalloproteinases (MMPs). It participates in a wide variety of cellular functions including lymphocyte activation, recirculation and homing, hematopoiesis, and tumor metastasis. CD44 is found in various splice formats and variations in CD44 are reported as cell surface markers for some breast and prostate cancer stem cells. It has also been seen as an indicator of increased survival time in epithelial ovarian cancer patients. CD44 variant isoforms are also relevant to the progression of head and neck squamous cell carcinoma.

CD47 is a membrane protein, which is involved in the increase in intracellular calcium concentration that occurs upon cell adhesion to extracellular matrix. The protein is also a receptor for the C-terminal cell binding domain of thrombospondin, and it may play a role in membrane transport and signal transduction. CD47 is a molecule found in many types of cancer and is used by bladder cancer cells to hide from normal scavenging by macrophages.

CD54 (also known as intracellular adhesion molecule 1) is a cell surface glycoprotein which is typically expressed on endothelial cells and cells of the immune system. It binds to integrins of type CD11a/CD18, or CD11b/CD18. CD54 is found on B-cell lymphoblastic lymphoma, mucosa associated lymphoid tissue lymphoma as well as other endothelial cancers as is implicated to play a role in metastasis.

CD55 (also known as complement decay accelerating factor) is a 70 kDa membrane protein that regulates the complement system on the cell surface. It prevents the assembly of the C3bBb complex (the C3-convertase of the alternative pathway) or accelerates the disassembly of preformed convertase, thus blocking the formation of the membrane attack complex. This glycoprotein is broadly distributed among hematopoietic and non-hematopoietic cells. It is found on many endothelial carcinomas including colorectal and prostate.

CD58 is a cell adhesion molecule expressed on Antigen Presenting Cells (APC), particularly macrophages. It binds to CD2 on T cells and is important in strengthening the adhesion between the T cells and Professional Antigen Presenting Cells. This adhesion occurs as part of the transitory initial encounters between T cells and Antigen Presenting Cells before T cell activation, when T cells are roaming the lymph nodes looking at the surface of APCs for peptide:MHC complexes. It is expressed on many lymphomas including B-cell lymphoblastic lymphoma and mucosa associated lymphoid tissue lymphoma.

CD59 inhibits the complement membrane attack complex by binding C5b678 and preventing C9 from binding and polymerizing. It is present on “self” cells to prevent complement from damaging them. It has a wide tissue distribution and has been implicated in breast and prostate cancers.

CD62L is a cell adhesion molecule found on lymphocytes. It belongs to the selectin family of proteins, which recognize sialylated carbohydrate groups. It is cleaved by ADAM17. It acts as a “homing receptor” for lymphocytes to enter secondary lymphoid tissues via high endothelial venules. It has been found on B cell lymphomas including chronic lymphocytic leukaemia as well as T cell lymphomas including adult T cell lymphoma.

CD95 (also known as Fas ligand) is a death receptor on the surface of cells that leads to programmed cell death (apoptosis). It is found on many cell types and has been implicated in many types of cancer including ovarian and colorectal carcinoma.

Accordingly, a first aspect of the invention provides an agent comprising:
(i) a T cell antigen, and
(ii) a binding partner for any of CD70, CD74, CD22, HLA-DR, CD23, CD30, CD43, CD44, CD47, CD54, CD55, CD58, CD59, CD62L or CD95,
wherein, following binding of the agent to a cell that expresses any of CD70, CD74, CD22, HLA-DR, CD23, CD30, CD43, CD44, CD47, CD54, CD55, CD58, CD59, CD62L or CD95, the agent is internalised and the T cell antigen is presented on the surface of the cell in a form that can be recognised by a T cell.

For the avoidance of doubt, when the binding partner is for CD70, the agent will bind to a cell that expresses CD70, and so on.

### T Cell Antigen

By a ‘T cell antigen’ we include the meaning of any antigen which can be presented to a T cell so as to elicit a T cell response. For example, the T cell antigen may be presented to a T cell by an MHC molecule or by a Group I CD1 molecule on the surface of the cell expressing any of CD70, CD74, CD22, HLA-DR, CD23, CD30, CD43, CD44, CD47, CD54, CD55, CD58, CD59, CD62L or CD95. Once the antigen is presented on the surface of the cell, the cell is recognised as foreign and becomes the target of T cells, some of which have the natural function of eliminating foreign cells either infected by foreign organisms such as viruses, fungi, bacteria, mycobacteria or protozoa, or which have become cancerous (eg malignant). Thus, it will be appreciated that the T cell antigen may be one that is capable of being presented by a molecule on an unwanted cell expressing any of CD70, CD74, CD22, HLA-DR, CD23, CD30, CD43, CD44, CD47, CD54, CD55, CD58, CD59, CD62L or CD95.

It will be appreciated that the T cell antigen is one that can elicit an existing T cell response in the subject to which the agent of the invention is administered. Typically, the T cell antigen is not one which generates a new primary T cell response for that antigen via cross-presentation in APCs. To put it another way, the T cell antigen is one to which a number of T cells in the subject are already sensitised to. Determining whether a subject's cells are sensitised to a given antigen can be done by contacting isolated peripheral mononuclear blood cells from the subject with the antigen and using standard assays for cell proliferation, as described further below and in the Examples.

In an embodiment, the agent of the invention is not one which generates a new T cell response specific for the T cell antigen contained in it. Accordingly, the invention includes an agent comprising a T cell antigen and a binding partner for any of CD70, CD74, CD22, HLA-DR, CD23, CD30, CD43, CD44, CD47, CD54, CD55, CD58, CD59, CD62L or CD95, wherein, following binding of the agent to a cell that expresses any of CD70, CD74, CD22, HLA-DR, CD23, CD30, CD43, CD44, CD47, CD54, CD55, CD58, CD59, CD62L or CD95, the agent is internalised and the T cell antigen is presented on the surface of the cell in a form that can be recognised by a T cell, and wherein the T cell antigen is capable of eliciting an existing T cell response in a subject.

By ‘T cell’, we include all types of T cell including CD4+, CD8+, γδ T cells, and NK-T cells. Preferably, the T cell is a CD4+ T cell, of which both helper and cytotoxic CD4+ T cells are known (Appay V (2004) Clin Exp Immunol 138(1): 10-13).

As is known in the art, the mechanism of antigen presentation will depend upon the type of T cell. It is understood that any presentation route may be used provided that the antigen elicits a T cell response. In other words, following internalisation of the agent of the invention, the T cell antigen must be capable of being presented on the surface of the cell such that it can elicit a T cell response. Preferably, the T cell antigen enters the MHC Class II processing machinery and is presented on an MHC Class II molecule, as described further below.

Preferably, the T cell antigen is an immunodominant antigen (eg an antigen that elicits an existing immunodominant response). By Immunodominant′ we include the meaning that the antigen elicits a T cell response with high magnitude, sensitivity, tissue homing characteristics and efficiency in killing antigen bearing cells. Generally, an immunodominant response comprises more than 0.1% of a subject's CD8⁺ or CD4⁺ T cells. Determining the extent of a T cell response for a given antigen can be done for example by contacting isolated peripheral mononuclear blood cells from the subject with the antigen and using standard assays for cell proliferation known in the art. Suitable assays for determining the extent of an immune response include ELISpot, intracellular cytokine staining, HLA-peptide tetramer staining, proliferation assay, activation assays (eg CD69), CD107 mobilisation assays or metabolic assays (eg MTT).

Examples of suitable T cell antigens include any of a peptide, a polypeptide, a phosphopeptide or a lipid such as a phospholipid or a sphingolipid, and further examples of each of these are provided below. Most preferably, the T cell antigen is a peptide or polypeptide.

When the T cell antigen is a peptide or polypeptide, typically it is one that is capable of being recognised by a T cell receptor when bound to an MHC Class II molecule. The T cell antigen may be an MHC Class II restricted antigen that binds only to MHC Class II molecules. It is appreciated that the antigen may bind only to particular variant MHC Class II molecules (e.g. natural variants found in particular subjects), or that the antigen may be capable of binding to any MHC Class II molecule (i.e. the antigen is promiscuous).

In one embodiment, the T cell antigen is capable of binding to a MHC Class II molecule such as any of HLA-DP, HLA-DQ or HLA-DR. Common MHC Class II types include DR1, DR3, DR4, DR7, DR52, DQ1, DQ2, DQ4, DQ8 and DP1. MHC Class II molecules are expressed on immune cells including antigen presenting cells such as dendritic cells, B cells and macrophages. Thus, when the T cell antigen is MHC Class II restricted, the agent of the invention may be used to treat conditions such as lymphomas or autoimmune diseases. However, it will be appreciated that MHC Class II molecules can be upregulated on non-immune cells (eg upon stimulation with IFN-gamma) and so other conditions may also be treated.

An example of a promiscuous peptide that may be used is the PADRE MHC Class-II epitope defined in Alexander et al (2000) The Journal of Immunology 164: 1625-1633; aKXVAAWTLKAAaZC (a=d-Alanine, X=L-cyclohexylalanine, Z=aminocaproic acid)) (SEQ ID No: 1). Since this epitope is artificial, it would first need to be introduced to the patient in a vaccine to generate an immune response prior to administering the agent of the invention. Another promiscuous peptide that may be used is the tetanus fragment C peptide.

Conveniently, the T cell antigen is an immunogenic peptide that is recognised by an MHC Class II molecule. Such peptides usually have a length of 9 to 22 amino acids. Preferably, the peptide is an immunodominant peptide.

Examples of immunodominant peptides include viral derived peptides that elicit endogenous anti-viral responses. Thus, the peptide may be derived from an endogenous virus such as Varicella-Zoster virus, Herpes simplex virus, cytomegalovirus, Epstein Barr virus, or influenza. Particularly preferred examples, which may be used in combination with any of the binding partners described herein (eg CD22 binding partner) include peptides derived from human cytomegalovirus (CMV or Human herpesvirus 5/HHV5) or Epstein-Barr Virus (EBV or HHV4); herpesviruses such as HHV1, HHV2 and HHV3; influenza virus A; influenza virus B; rhinovirus; adenovirus; and Hepadnaviridae, specific examples of which are given below.

For human cytomegalovirus (HHV5) the immunodominant antigens are well characterised (see Sylwester A W et al J Exp Med. 2005 Sep. 5; 202(5):673-85, incorporated herein by reference), and so an antigen described in Sylwester et al may be used in the present invention. In particular, Sylester et al synthesised consecutive 15mer peptides, overlapping by 10 amino acids, for 213 predicted human CMV proteins. This generated 13,687 peptides that were arranged in ORF or sub-ORF specific mixes. Peptides derived from ORFs UL55 (gB), UL 83 (pp65), UL 86, UL 99 (pp28), UL 122 (IE2), UL 36, UL 48, UL32 (pp150), UL 113, IRS-1, UL 123 (IE1), UL25, UL 141, UL 52 and UL 82 (pp71) were found to elicit the most CD 4+ T cell responses, and so it is particularly preferred if the peptide is derived from one of these ORFs.

Particular cytomegalovirus T cell antigens that may be used are listed below.

CD4+ T cell epitopes for cytomegalovirus antigens such as pp65 include PQYSEHPTFTSQYRIQ (SEQ ID No: 1), FTSQYRIQGKLEYRHT (SEQ ID No: 2), LLQTGIHVRVSQPSL (SEQ ID No: 43), NPQPFMRPHERNGFT (SEQ ID No: 4), EPDVYYTSAFVFPTK (SEQ ID No: 5), IIKPGKISHIMLDVA (SEQ ID No: 6), AGILARNLVPMVATV (SEQ ID No: 7), KYQEFFWDANDIYRI (SEQ ID No: 8); for gB they include DYSNTHSTRYV (SEQ ID No: 9), CMLTITTARSKYPYH (SEQ ID No: 10), and VFETSGGLWFWQGI (SEQ ID No: 11); for IE1 they include VRVDMVRHRIKEHMLKKYTQ (SEQ ID No: 12) and NYIVPEDKREMWMACIKELH (SEQ ID No: 13); and for gH they include HELLVLVKKAQL (SEQ ID No: 14). For Epstein Barr Virus (EBV or HHV4), immunodominant proteins are also well characterised and are provided in Hislop A D et al Annu Rev Immunol. 2007; 25:587-617 (incorporated herein by reference). A list of suitable T cell epitopes, adapted from Hislop et al is provided below.

TABLE 3
CD4+ T cell epitopes identified in EBV
lytic and latent cycle proteins
(adapted from Hislop et al)

| | | | |
|---|---|---|---|
| EBV | Epitope | Epitope sequence | HLA |
| Antigen | coordinates | (SEQ ID No) | restriction |

Latent cycle proteins

| | | | |
|---|---|---|---|
| EBNA1 | 71-85 | RRPQKRPSCIGCKGT (15) | |
| | 403-417 | RPFFHPVGEADYFEY (16) | |
| | 429-448 | VPPGAIEQGPADDPGEGPST (17) | |
| | 434-458 | IEQGPTDDPGEGPSTGPRGQGDGGR (18) | |
| | 455-469 | DGGRRKKGGWFGRHR (19) | |
| | 474-493 | SNPKFENIAEGLRVLLARSH (20) | |
| | 475-489 | NPKFENIAEGLRALL (21) | |
| | 479-498 | ENIAEGLRVLLARSHVERTT (22) | DQ7 |
| | 481-500 | IAEGLRALLARSHVERTTDE (23) | DQ2/3 |
| | 485-499 | LRALLARSHVERTTD (24) | |
| | 499-523 | EEGNWVAGVFVYGGSKTSLYNLRRG (25) | |
| | 509-528 | VYGGSKTSLYNLRRGTALAI (26) | DR11 |
| | 515-528 | TSLYNLRRGTALAI (27) | DR1 |
| | 518-530 | YNLRRGTALAIPQ (28) | DP3 |
| | 519-533 | NLRRGRTALAIPQCRL (29) | |
| | 519-543 | EEGNWVAGVFVYGGSKTSLYNLRRG (30) | |
| | 527-541 | AIPQCRLTPLSRLPF (31) | DR13 |
| | 529-543 | PQCRLTPLSRLPFGM (32) | DR14 |
| | 544-563 | APGPGPQPLRESIVCYFM (S43)(33) | |
| | 549-568 | PQPGPLRESIVCYFMVFLQT (S44)(34) | |
| | 551-570 | PGPLRESIVCYFMVFLQTHI (35) | DR1 |
| | 554-573 | LRESIVCYFMVFLQTHIFAE (36) | |
| | 554-578 | LRESIVCYFMVFLQTHIFAEVLKDA (37) | |
| | 561-573 | YFMVFLQTHIFAE (38) | DR11,12,13 |
| | 563-577 | MVFLQTHIFAEVLKD (39) | DR15 |
| | 564-583 | VFLQTHIFAEVLKDAIKDL (40) | DP5 |
| | 574-593 | VLKDAIKDLVMTKPAPTCNI (41) | |
| | 589-613 | PTCNIKVTVCSFDDGVDLPPWFPPM (42) | |
| | 594-613 | RVTVCSFDDGVDLPPWFPPM (43) | |
| | 607-619 | PPWFPPMVEGAAA (44) | DQ2 |
| EBNA2 | 11-30 | GQTYHLIVDTLALHGGQTYH (45) | DR4 |
| | 46-65 | IPLTIFVGENTGVPPPLPPP (46) | |
| | 131-150 | MRMLWMANYIVRQSRGDRGL (47) | |
| | 206-225 | LPPATLVPPRPTRPTTLPP (48) | |
| | 276-295 | PRSPTVFYNIPPMPLPPSQL (49) | DR7,52a,52b,52c |
| | 280-290 | TVFYNIPPMPL (50) | DQ2/DQ7 |
| | 301-320 | PAQPPPGVINDQQLHHLPSG (51) | DR17 |
| EBNA3A | 364-383 | EDLPCIVSRGGPKVKRPPIF (52) | DR15 |
| | 780-799 | GPWVPEQWMFQGAPPSQGTP (53) | DR1 |
| | 649-668 | QVADWRAPGVPAMQPQYF (54) | |
| EBNA3B | | | |
| EBNA3C | 66-80 | NRGWMQRIRRRRRR (55) | |
| | 100-119 | PHDITYPYTARNIRDAACRAV (56) | DR16 |
| | 141-155 | ILCFVMAARQRLQDI (57) | DR13 |
| | 386-400 | SDDELPYIDPNMEPV (58) | DQ5 |
| | 401-415 | QQRPVMFVSRVPAKK (59) | |
| | 546-560 | QKRAAPPTVSPSDTG (60) | |
| | 586-600 | PPAAGPPAAGPRILA (61) | |
| | 626-640 | PPWRMFMRERQLPQ (62) | |
| | 649-660 | PQCFWEMRAGREITQ (63) | |
| | 741-760 | PAPQAPYQGYQEPPAPQAPY (64) | DR1/DR4 |
| | 916-930 | PSMPFASDYSQGAFT (65) | |
| | 961-986 | AQEILSDNSEISVFPK (66) | |
| LMP1 | 11-30 | GPPRPPLGPPLSSSIGLALL (67) | DR7 & DR9 |
| | 130-144 | LWRLGATIWQLLAFF (68) | |
| | 181-206 | LIWMYYHGPRHTDEHHHDDS (69) | DR16 |
| | 206-225 | QATDDSSHESDSNSNEGRHH (70) | DQ2 |
| | 211-236 | SSHESDSNSNEGRHHLLVSG (71) | DQB1*0601 |
| | 212-226 | SGHESDSNSNEGRHHH (72) | |
| | 340-354 | TDGGGGHSHDSGHGG (73) | |
| LMP2 | 73-87 | DYQPLGTQDQSLYLG (74) | DR4 or DR16 |
| | 149-163 | STVVTATGLALSLLL (75) | |
| | 169-182 | SSYAAAQRKLLTPV (76) | |
| | 189-208 | VTFFAICLTVVRIEDPPFNSI (77) | DRB1*0901 |
| | 194-213 | ICLTWRIEDPPFNSILFALL (78) | DRB1*1001 |
| | 224-243 | VLVMLVLLILAYRRRWRRLT (79) | |
| | 385-398 | STEFIPNLFCMLLL (80) | |
| | 419-438 | TYGPVFMSLGGLLTMVAGAV (81) | DQB1*0601 |

Lytic Cycle Proteins

| | | | |
|---|---|---|---|
| BHRF1 | 171-189 | AGLTLSLLVICSYLFISRG (82) | DR2 |
| | 122-133 | PYYVVDLSVRGM (83) | DR4 |
| | 45-57 | TVVLRYHVLLEEI (84) | DR4 |
| BZLF1 | 174-188 | ELEIKRYKNRVASRK (85) | DR13 |
| | 207-221 | KSSENDRLRLLLKQM (86) | DQB1*0402 |
| BLLF1 | 61-81 | LDLFGQLTPHTKAVYQPRGA (87) | DRw15 |
| (gp350) | 65-79 | FGQLTPHTKAVYQPR (88) | DRB1*1301 |
| | 130-144 | VYFQDVFGTMWCHHA (89) | DQB1*0402 |
| | 163-183 | DNCNSTNITAWRAQGLDVTL (90) | DRw11 |
| BALF4 | 482-496 | AWCLEQKRQNMVLRE (91) | DPB1*1301 |
| (gp110) | 575-589 | DNEIFLTKKMTEVCQ (92) | DRB1*0801 |

It is appreciated that the T cell antigen (e.g. peptide) may be one derived from a live vaccine such as Measles, Mumps, Rubella (MMR) or HHV3; or one derived from intracellular bacteria such as mycobacteria, particularly those evoked through immunization with BCG. Such peptides are well known in the art. Similarly, the T cell antigen (e.g. peptide) may be derived from the tetanus toxoid such as P2, P4 or P30. Thus, it will be understood that the T cell antigen (e.g. peptide) may be one that elicits an existing immune response in a subject that has been generated by prior vaccination against an infectious agent. It follows that in order to increase the number of T cells sensitised to a T cell antigen, it may be desirable to vaccinate or boost a subject with a vaccine that comprises the T cell antigen. For example, the subject may be vaccinated with a tetanus toxin, before being administered the agent of the invention comprising the relevant T cell antigen.

It will be appreciated that because many people are vaccinated in childhood with these vaccines, they are likely to contain T cells which are sensitized to these T cell antigens. Thus, in one embodiment the T cell antigen is one which is found in a childhood vaccine, preferably one that is routinely used such as MMR, measles, BCG, yellow fever, polio, V2V and influenza.

Although not preferred, the T cell antigen (eg peptide) may also be one that elicits an existing immune response in a subject that has been generated by exposing that subject's T cells to the antigen in vitro.

Peptides can be produced by well known chemical procedures, such as solution or solid-phase synthesis, or semi-synthesis in solution beginning with protein fragments coupled through conventional solution methods as is known in the art. Alternatively, the peptide can be synthesised by established methods including recombinant methods.

Although it is preferred that the T cell antigen is a polypeptide or peptide, it is known that other antigens are also capable of eliciting immune responses and so have utility in the present invention. For example, γδ T cells do not recognise MHC-associated peptide antigens and are not MHC restricted. Some γδ T cell clones recognise small phosphorylated molecules, pyrophosphorylated compounds (eg HMBPP (E-4-hydroxy-3-methyl-but-2-enyl-pyrophosphate) and IPP (isopentenyl pyrophosphate)), alkyl amines or lipids (e.g. phosphorylated lipids) that may be presented by ‘non-classical’ class I MHC-like molecules called CD1 molecules. Similarly, NK-T cells (e.g. Vα24Vβ11 cells) recognise lipids (e.g. ceramides such as a-gal-ceramide) bound to CD1 molecules. Thus, the T cell antigen may be any of these molecules that are known to elicit a T cell response. Of course, the T cell antigen must be one that is capable of being presented on any of these molecules following internalisation of the agent into the cell.

When the agent is used as described below to treat autoimmune or allergic diseases, it will be appreciated that the T cell antigen may be an autoantigen or allergen respectively. In this way the immune response that is contributing to the disorder is redirected to unwanted cells so as to combat the disorder.

It is appreciated that the T cell antigen may be chemically modified provided that it is still capable of eliciting a T cell response. Such chemical modification may include, for instance, the addition of a metal such as nickel, since it has been shown that in certain allergic patients there are T cells which recognise a peptide with a bound nickel atom (Romagnoli et al 1991, EMBO J 10: 1303-1306). The T cell antigen can also be modified by an organic molecule which enhances the immunogenicity (Romero et al 1993, J Immunol 150: 3825-3831). Other modifications include phosphorylation, acetylation, alkylation, acylation, amidation, glycosylation, methylation, citrulination, nitration, sulphation and hydroxylation, forming salts with acids or bases, forming an ester or amide of a terminal carboxyl group, and attaching amino acid protecting groups such as N-t-butoxycarbonal.

When the T cell antigen is a peptide, it is appreciated that it may comprise naturally occurring amino acids encoded by DNA, and/or one or more non-natural amino acids, including amino acids in the “D” isomeric form or incorporating the use of N-methylated amino acids or beta amino acids or peptoids, provided that it is recognised by the corresponding T cell. Thus, the peptide may be a peptide ‘mimetic’ ie peptidomimetic which mimics the structural features of any of the peptides mentioned above. For example, the T cell antigen may be a retro-inverso peptide.

Similarly, the T cell antigen, when a peptide, may be a mimotope, ie a peptide composed of natural or non-natural amino acids that mimics the structure of the natural epitope. Mimotopes often stimulate T cells more potently.

Preferably, the T cell antigens are substantially non-toxic in the absence of T lymphocytes. By ‘substantially non-toxic’ we mean that the antigens have considerably lower or preferably no detectable toxicity, compared to toxins such as Pseudomonas exotoxin.

The skilled person will be able to identify further T cell antigens that may be used in the invention using the database available at http://www.immuneepitope.org (Vita R, Zarebski L, Greenbaum J A, Emami H, Hoof I, Salimi N, Damle R, Sette A, Peters B. The immune epitope database 2.0. Nucleic Acids Res. 2010 January; 38(Database issue):D854-62. Epub 2009 Nov. 11).

### Binding Partner

By ‘binding partner of any of CD70, CD74, CD22, HLA-DR, CD23, CD30, CD43, CD44, CD47, CD54, CD55, CD58, CD59, CD62L or CD95’, we include the meaning of any molecule that binds to any of CD70, CD74, CD22, HLA-DR, CD23, CD30, CD43, CD44, CD47, CD54, CD55, CD58, CD59, CD62L or CD95 respectively. In this way, the agent of the invention can bind to the surface of cells that express any of CD70, CD74, CD22, HLA-DR, CD23, CD30, CD43, CD44, CD47, CD54, CD55, CD58, CD59, CD62L or CD95.

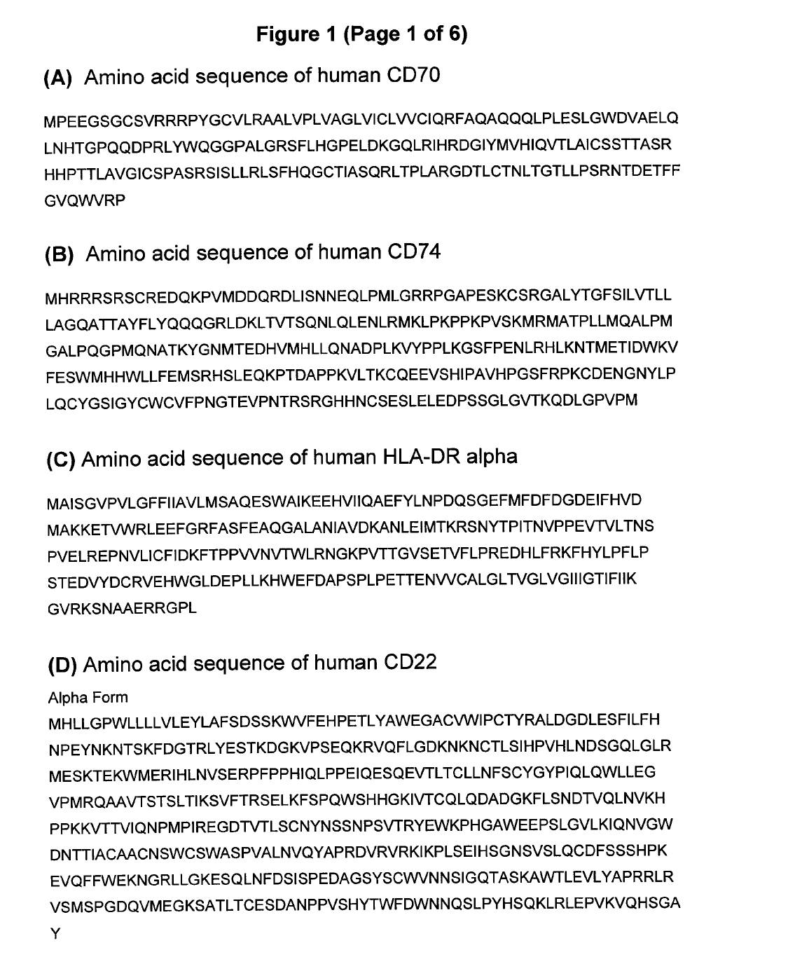

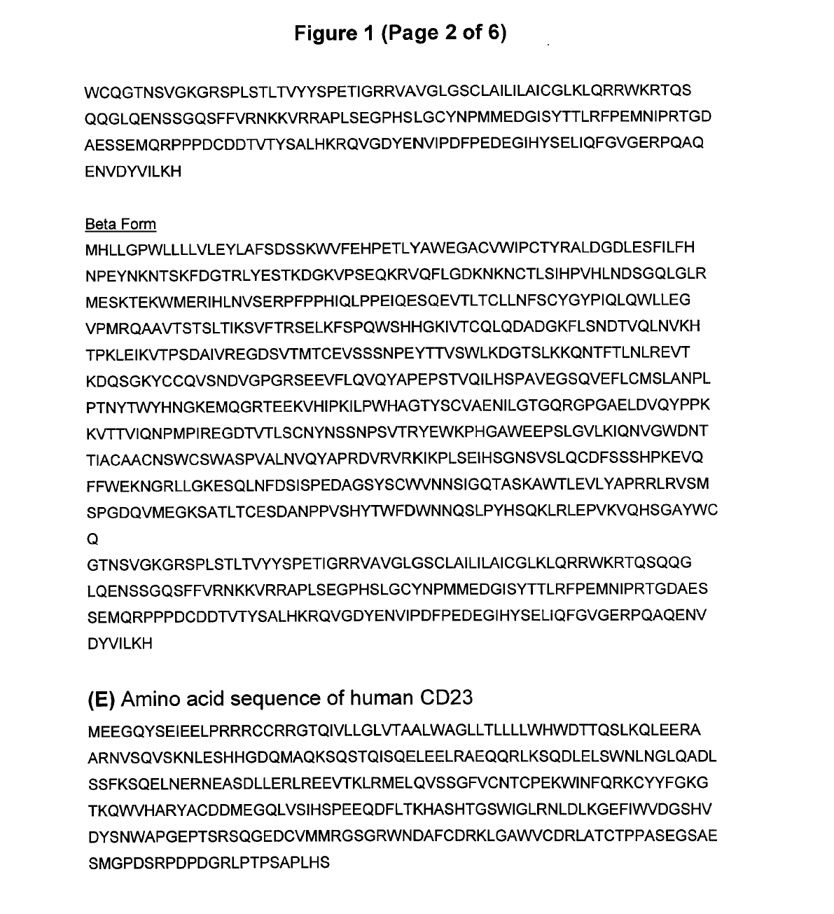

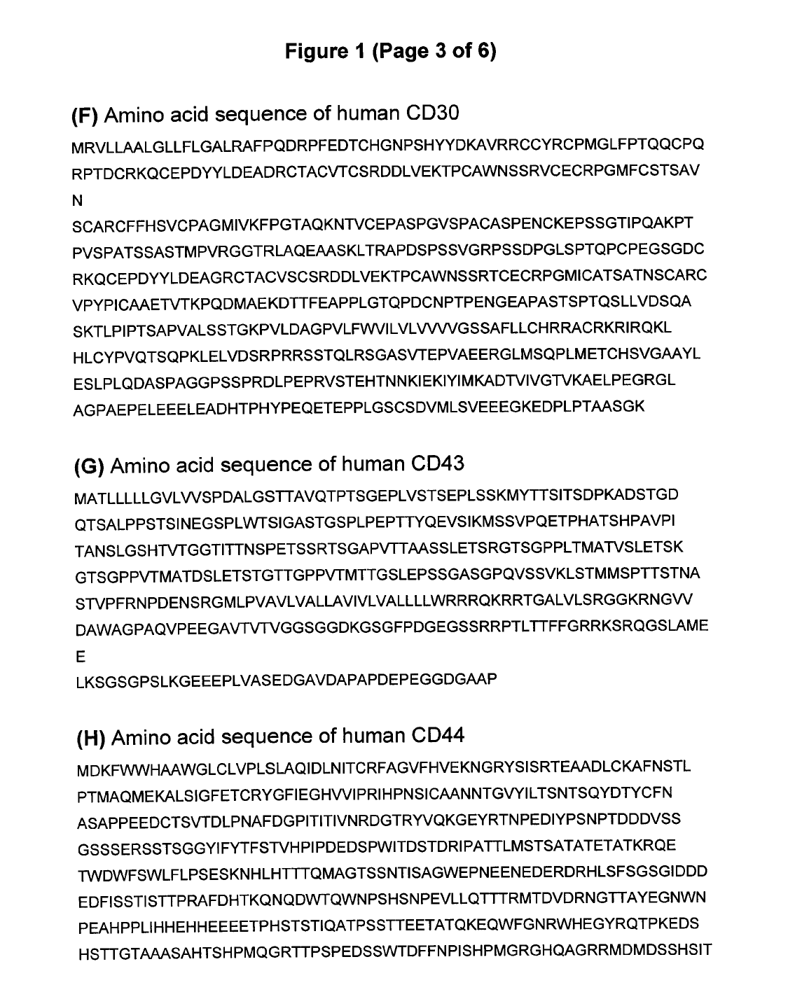

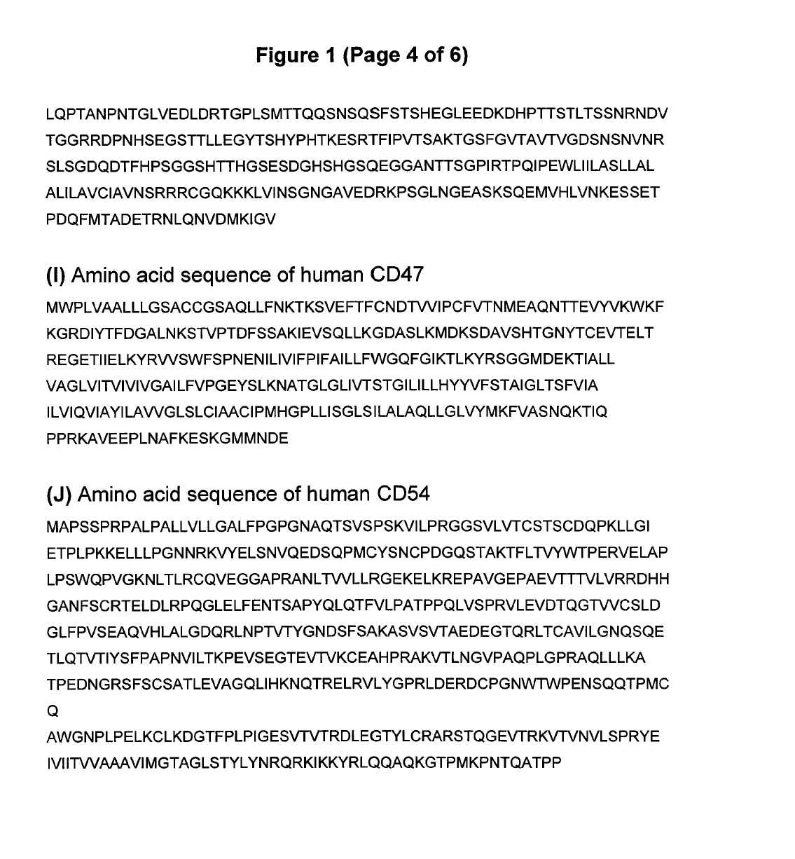

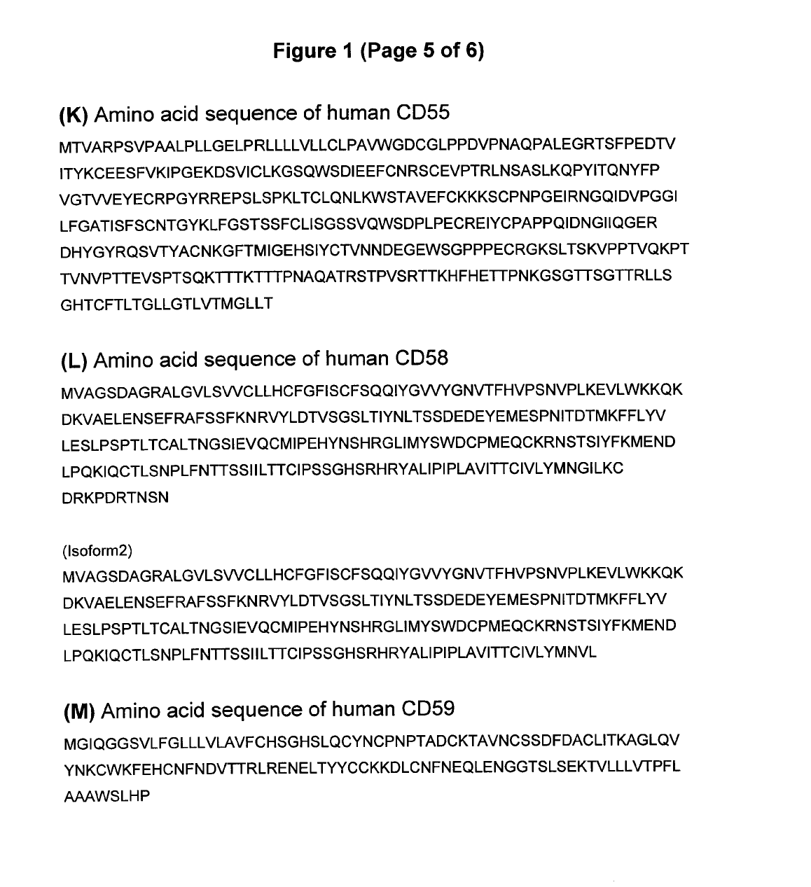

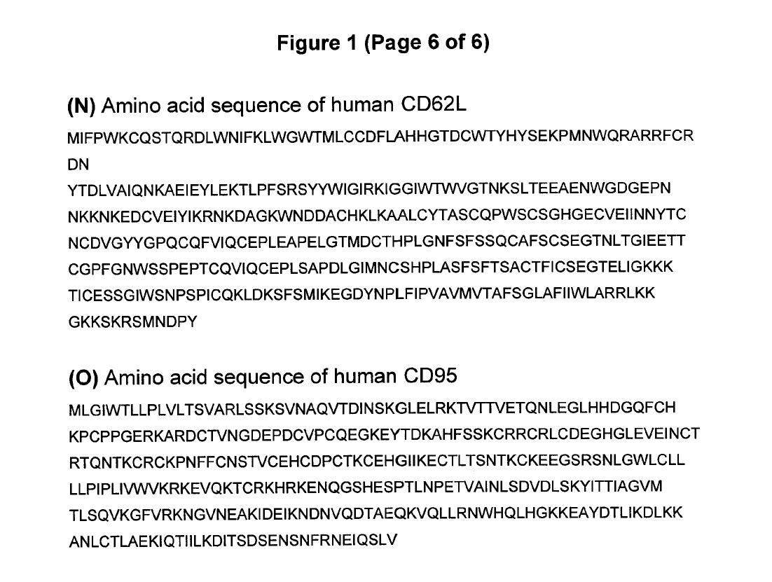

By ‘CD70’ we include human CD70, the amino sequence of which is provided in FIG. 1A and which has Accession Number P32970. However, it is well known that certain polypeptides are polymorphic, and it is appreciated that some natural variation of this sequence may occur. Thus, in an embodiment, the binding partner of CD70 is not limited to the binding partner of human CD70 having the sequence listed in FIG. 1A, but includes binding partners to naturally occurring variants thereof in which one or more of the amino acid residues have been replaced with another amino acid. The binding partner of CD70 also includes binding partners of CD70 in other species which have an orthologous sequence to that in FIG. 1A, for example CD70 from other mammals such as horse, dog, pig, cow, sheep, rat, mouse, guinea pig or a primate.

By ‘CD74’ we include human CD74, the amino sequence of which is provided in FIG. 1B and which has Accession Number P04233. However, it is well known that certain polypeptides are polymorphic, and it is appreciated that some natural variation of this sequence may occur. Thus, in an embodiment, the binding partner of CD74 is not limited to the binding partner of human CD74 having the sequence listed in FIG. 1B, but includes binding partners to naturally occurring variants thereof in which one or more of the amino acid residues have been replaced with another amino acid. The binding partner of CD74 also includes binding partners of CD74 in other species which have an orthologous sequence to that in FIG. 1B, for example CD74 from other mammals such as horse, dog, pig, cow, sheep, rat, mouse, guinea pig or a primate.

By ‘CD22’ we include human CD22, the amino sequence of which is provided in FIG. 1B and which has Accession Number P20273. However, it is well known that certain polypeptides are polymorphic, and it is appreciated that some natural variation of this sequence may occur. Thus, in an embodiment, the binding partner of CD22 is not limited to the binding partner of human CD22 having the sequence listed in FIG. 1C, but includes binding partners to naturally occurring variants thereof in which one or more of the amino acid residues have been replaced with another amino acid. The binding partner of CD22 also includes binding partners of CD22 in other species which have an orthologous sequence to that in FIG. 1C, for example CD22 from other mammals such as horse, dog, pig, cow, sheep, rat, mouse, guinea pig or a primate.

By ‘HLA-DR’ we include human HLA-DR, the amino sequence of which is provided in FIG. 1D and which has Accession Number Q29769. However, it is well known that certain polypeptides are polymorphic, and it is appreciated that some natural variation of this sequence may occur. Thus, in an embodiment, the binding partner of HLA-DR is not limited to the binding partner of human HLA-DR having the sequence listed in FIG. 1D, but includes binding partners to naturally occurring variants thereof in which one or more of the amino acid residues have been replaced with another amino acid. The binding partner of HLA-DR also includes binding partners of HLA-DR in other species which have an orthologous sequence to that in FIG. 1D, for example HLA-DR from other mammals such as horse, dog, pig, cow, sheep, rat, mouse, guinea pig or a primate.

By ‘CD23’ we include human CD23, the amino sequence of which is provided in FIG. 1E and which has Accession Number P06734. However, it is well known that certain polypeptides are polymorphic, and it is appreciated that some natural variation of this sequence may occur. Thus, in an embodiment, the binding partner of CD23 is not limited to the binding partner of human CD23 having the sequence listed in FIG. 1E, but includes binding partners to naturally occurring variants thereof in which one or more of the amino acid residues have been replaced with another amino acid. The binding partner of CD23 also includes binding partners of CD23 in other species which have an orthologous sequence to that in FIG. 1E, for example CD23 from other mammals such as horse, dog, pig, cow, sheep, rat, mouse, guinea pig or a primate.

By ‘CD30’ we include human CD30, the amino sequence of which is provided in FIG. 1F and which has Accession Number P28908. However, it is well known that certain polypeptides are polymorphic, and it is appreciated that some natural variation of this sequence may occur. Thus, in an embodiment, the binding partner of CD30 is not limited to the binding partner of human CD30 having the sequence listed in FIG. 1F, but includes binding partners to naturally occurring variants thereof in which one or more of the amino acid residues have been replaced with another amino acid. The binding partner of CD30 also includes binding partners of CD30 in other species which have an orthologous sequence to that in FIG. 1F, for example CD30 from other mammals such as horse, dog, pig, cow, sheep, rat, mouse, guinea pig or a primate.

By ‘CD43’ we include human CD43, the amino sequence of which is provided in FIG. 1G and which has Accession Number P16150. However, it is well known that certain polypeptides are polymorphic, and it is appreciated that some natural variation of this sequence may occur. Thus, in an embodiment, the binding partner of CD43 is not limited to the binding partner of human CD43 having the sequence listed in FIG. 1G, but includes binding partners to naturally occurring variants thereof in which one or more of the amino acid residues have been replaced with another amino acid. The binding partner of CD43 also includes binding partners of CD43 in other species which have an orthologous sequence to that in FIG. 1G, for example CD43 from other mammals such as horse, dog, pig, cow, sheep, rat, mouse, guinea pig or a primate.

By ‘CD44’ we include human CD44, the amino sequence of which is provided in FIG. 1H and which has Accession Number P16070. However, it is well known that certain polypeptides are polymorphic, and it is appreciated that some natural variation of this sequence may occur. Thus, in an embodiment, the binding partner of CD44 is not limited to the binding partner of human CD44 having the sequence listed in FIG. 1H, but includes binding partners to naturally occurring variants thereof in which one or more of the amino acid residues have been replaced with another amino acid. The binding partner of CD44 also includes binding partners of CD44 in other species which have an orthologous sequence to that in FIG. 1H, for example CD44 from other mammals such as horse, dog, pig, cow, sheep, rat, mouse, guinea pig or a primate.

By ‘CD47’ we include human CD47, the amino sequence of which is provided in FIG. 11 and which has Accession Number P08722. However, it is well known that certain polypeptides are polymorphic, and it is appreciated that some natural variation of this sequence may occur. Thus, in an embodiment, the binding partner of CD47 is not limited to the binding partner of human CD47 having the sequence listed in FIG. 1I, but includes binding partners to naturally occurring variants thereof in which one or more of the amino acid residues have been replaced with another amino acid. The binding partner of CD47 also includes binding partners of CD47 in other species which have an orthologous sequence to that in FIG. 1I, for example CD47 from other mammals such as horse, dog, pig, cow, sheep, rat, mouse, guinea pig or a primate.

By ‘CD54’ we include human CD54, the amino sequence of which is provided in FIG. 1J and which has Accession Number P05362. However, it is well known that certain polypeptides are polymorphic, and it is appreciated that some natural variation of this sequence may occur. Thus, in an embodiment, the binding partner of CD54 is not limited to the binding partner of human CD54 having the sequence listed in FIG. 1J, but includes binding partners to naturally occurring variants thereof in which one or more of the amino acid residues have been replaced with another amino acid. The binding partner of CD54 also includes binding partners of CD54 in other species which have an orthologous sequence to that in FIG. 1J, for example CD54 from other mammals such as horse, dog, pig, cow, sheep, rat, mouse, guinea pig or a primate.

By ‘CD55’ we include human CD55, the amino sequence of which is provided in FIG. 1K and which has Accession Number P08174. However, it is well known that certain polypeptides are polymorphic, and it is appreciated that some natural variation of this sequence may occur. Thus, in an embodiment, the binding partner of CD55 is not limited to the binding partner of human CD55 having the sequence listed in FIG. 1K, but includes binding partners to naturally occurring variants thereof in which one or more of the amino acid residues have been replaced with another amino acid. The binding partner of CD55 also includes binding partners of CD55 in other species which have an orthologous sequence to that in FIG. 1K, for example CD55 from other mammals such as horse, dog, pig, cow, sheep, rat, mouse, guinea pig or a primate.

By ‘CD58’ we include human CD58, the amino sequence of which is provided in FIG. 1L and which has Accession Number P19256. However, it is well known that certain polypeptides are polymorphic, and it is appreciated that some natural variation of this sequence may occur. Thus, in an embodiment, the binding partner of CD58 is not limited to the binding partner of human CD58 having the sequence listed in FIG. 1L, but includes binding partners to naturally occurring variants thereof in which one or more of the amino acid residues have been replaced with another amino acid. The binding partner of CD58 also includes binding partners of CD58 in other species which have an orthologous sequence to that in FIG. 1L, for example CD58 from other mammals such as horse, dog, pig, cow, sheep, rat, mouse, guinea pig or a primate.

By ‘CD59’ we include human CD59, the amino sequence of which is provided in FIG. 1M and which has Accession Number P13987. However, it is well known that certain polypeptides are polymorphic, and it is appreciated that some natural variation of this sequence may occur. Thus, in an embodiment, the binding partner of CD59 is not limited to the binding partner of human CD59 having the sequence listed in FIG. 1M, but includes binding partners to naturally occurring variants thereof in which one or more of the amino acid residues have been replaced with another amino acid. The binding partner of CD59 also includes binding partners of CD59 in other species which have an orthologous sequence to that in FIG. 1M, for example CD59 from other mammals such as horse, dog, pig, cow, sheep, rat, mouse, guinea pig or a primate.

By ‘CD62L’ we include human CD62L, the amino sequence of which is provided in FIG. 1N and which has Accession Number P14151. However, it is well known that certain polypeptides are polymorphic, and it is appreciated that some natural variation of this sequence may occur. Thus, in an embodiment, the binding partner of CD62L is not limited to the binding partner of human CD62L having the sequence listed in FIG. 1N, but includes binding partners to naturally occurring variants thereof in which one or more of the amino acid residues have been replaced with another amino acid. The binding partner of CD62L also includes binding partners of CD62L in other species which have an orthologous sequence to that in FIG. 1N, for example CD62L from other mammals such as horse, dog, pig, cow, sheep, rat, mouse, guinea pig or a primate.

By ‘CD95’ we include human CD95, the amino sequence of which is provided in FIG. 10 and which has Accession Number P25445. However, it is well known that certain polypeptides are polymorphic, and it is appreciated that some natural variation of this sequence may occur. Thus, in an embodiment, the binding partner of CD95 is not limited to the binding partner of human CD95 having the sequence listed in FIG. 1O, but includes binding partners to naturally occurring variants thereof in which one or more of the amino acid residues have been replaced with another amino acid. The binding partner of CD95 also includes binding partners of CD95 in other species which have an orthologous sequence to that in FIG. 1O, for example CD95 from other mammals such as horse, dog, pig, cow, sheep, rat, mouse, guinea pig or a primate.

As described further below, the agents of the invention may be administered to subjects for use in medicine. With respect to the subject to which the agent is administered, it is preferred that the agent comprises a binding partner that binds to any of CD70, CD74, CD22, HLA-DR, CD23, CD30, CD43, CD44, CD47, CD54, CD55, CD58, CD59, CD62L or CD95 of that species. For example, when the subject is a human, the agent comprises a binding partner of human any of CD70, CD74, CD22, HLA-DR, CD23, CD30, CD43, CD44, CD47, CD54, CD55, CD58, CD59, CD62L or CD95, and so on.

Preferably, the binding partner binds selectively to any of CD70, CD74, CD22, HLA-DR, CD23, CD30, CD43, CD44, CD47, CD54, CD55, CD58, CD59, CD62L or CD95. For example, it is preferred if the binding partner of any of CD70, CD74, CD22, HLA-DR, CD23, CD30, CD43, CD44, CD47, CD54, CD55, CD58, CD59, CD62L or CD95 has a K_{d} value (dissociation constant) which is at least five or ten times lower (i.e. higher affinity) than for at least one other entity expressed by that cell, and preferably more than 100 or 500 times lower. More preferably, the binding partner of any of CD70, CD74, CD22, HLA-DR, CD23, CD30, CD43, CD44, CD47, CD54, CD55, CD58, CD59, CD62L or CD95 has a K_{d} value more than 1000 or 5000 times lower than for at least one other entity expressed by that cell. K_{d} values can be determined readily using methods well known in the art.

The binding partner may be any of a polypeptide, a peptide, a small molecule or a peptidomimetic.

In a preferred embodiment, the binding partner is an antibody that binds to CD70, CD74, CD22, HLA-DR, CD23, CD30, CD43, CD44, CD47, CD54, CD55, CD58, CD59, CD62L or CD95.

The binding partner may be an anti-CD70 antibody such as any of BU69 or SGN-70 or SGN-75 or a commercial antibody from eBioscience (anti-human CD74 purified, clone LN2) (Epstein et al, 1984, Immunol. 133(2): 1028; Lamb et al, 1991, PNAS 88(14): 5998).

The binding partner may be an anti-CD74 antibody such as milatuzumab (Becker-Herman et al, 2005, Mol. Biol. Cell. 16(11): 5061).

The binding partner may be an anti-CD22 antibody such as Epratuzumab (Stein R. et al, Cancer Immunol Immunother 37: 293-298 (October 1993).

The binding partner may be an anti-HLA-DR antibody such as a commercial antibody from eBioscience (anti-human HLA-DR, clone L243) (Brodsky F M. A. Immunogenetics 1984; 19(3): 179-94; Engleman E G, Warnke R, Fox R I, Dilley J, Benike C J, Levy R. Proc Natl Acad Sci USA 1981 March; 78(3): 1791-5.).

The binding partner may be an anti-CD23 antibody such as a commercial antibody from eBioscience (anti-human CD23 purified, clone EBVCS2) (Knapp, W., B. Dorken, et al eds. (1989) Leucocyte Typing IV: White Cell Differentiation Antigens. Oxford University Press. New York; McMichael, A. J., P. C. L. Beverly, et al eds. (1987) Leucocyte Typing III: White Cell Differentiation Antigens. Oxford University Press. New York; Bernard, A., et al eds. (1981) Leukocyte Typing. Springer-Verlag.

The binding partner may be an anti-CD30 antibody such as a commercial antibody from eBioscience (anti-human CD30 purified, clone BerH2) Tamiolakis D, et al Int J Biol Sci 2005; 1: 135-140; Polski J M, Janney CG. Ber-H2 Mod Pathol. 1999 September; 12(9): 903-6; Horie R, Watanabe, T. J Immunol 1998; 10: 457-470.)

The binding partner may be an anti-CD43 antibody such as a commercial antibody from eBioscience (anti-human CD43 purified, clone eBio84-3C1) (Borche L, Lozano F, Vilella R, Vives J. Eur J Immunol. 1987 October; 17(10):1523-6; Schlossman, S., L. Bloumsell, et al eds. 1995. Leucocyte Typing V: White Cell Differentiation Antigens. Oxford University Press. New York.).

The binding partner may be an anti-CD44 antibody such as a commercial antibody from eBioscience (anti-human CD44 purified, clone IM7) (Trowbridge, I. S., J. Lesley, at al 1982. Immunogenetics 15(3): 299-312; Lesley, J. and I. S. Trowbridge 1982. Immunogenetics 15(3): 313-20; Maiti A, Maki G, Johnson P. Science. 1998. Oct. 30; 282(5390): 941-3.).

The binding partner may be an anti-CD47 antibody such as a commercial antibody from eBioscience (anti-human CD47 purified, clone B6H12) (Grimbert P, Bouguermouh S, et al J Immunol. 2006 Sep. 15; 177(6): 3534-41; Lagadec P, Dejoux O, et al 2003 Jun. 15; 101(12): 4836-43.).

The binding partner may be an anti-CD54 antibody such as a commercial antibody from eBioscience (anti-human CD54 purified, clone eBio KAT1) (Lehmann J C, et al J Immunol. 2003 Sep. 1; 171(5): 2588-93; Arai K, et al Int J Pancreatol. 1999 August; 26(1): 23-31).

The binding partner may be an anti-CD55 antibody such as a commercial antibody from eBioscience (anti-human CD55 purified, clone 143-30) (Knapp, W., B. Dorken, et al eds. (1989). Leucocyte Typing IV: White Cell Differentiation Antigens. Oxford University Press. New York).

The binding partner may be an anti-CD58 antibody such as a commercial antibody from eBioscience (anti-human CD58 purified, clone TS2/9) (Ariel O, et al, Cellular Signaling 2009: 21: 1100-1108; Osborn L, et al J. Exp. Med. January 1995; 181: 429-434.).

The binding partner may be an anti-CD59 antibody such as a commercial antibody from eBioscience (anti-human CD59 purified, clone OV9A2) (Alegretti A P et al Cell Immunol. 2010; 265(2): 127-32; Deckert M, et al Eur J Immunol. 1992 November; 22(11): 2943-7).

The binding partner may be an anti-CD62L antibody such as a commercial antibody from eBioscience (anti-human CD59 purified, clone DREG-56) (Jutila M A, et al J. Immunol, August 15; 169(4): 1768-73; Schlossman, S., L. Bloumsell et al eds. 1995. Leucocyte Typing V: White Cell Differentiation Antigens. Oxford University Press. New York).

The binding partner may be an anti-CD95 antibody such as a commercial antibody from eBioscience (anti-human CD59 purified, clone APO-1-1) (Rajasagi, M et al Journal of Leukocyte Biology 2009; 85: 251-261; Fluhr, H et al Journal of Cell Science 2007; 120: 4126-4133.).

Alternatively, the binding partner may be any molecule or part thereof that specifically binds, in a non-immune sense, to any of CD70, CD74, CD22, HLA-DR, CD23, CD30, CD43, CD44, CD47, CD54, CD55, CD58, CD59, CD62L or CD95. Thus, the specific binding partner may be any of a hormone, a growth factor, a cytokine, or a receptor ligand (e.g. agonist or antagonist).

CD70 is known to bind to CD27, and so in one embodiment, the binding partner of CD70 is CD27.

CD74 is known to bind to macrophage migration inhibitory factor (MIF), and so in one embodiment, the binding partner of CD74 is MIF.

CD23 is known to bind to IgE, and so in one embodiment, the binding partner of CD23 is IgE.

CD30 is known to bind to CD30L, and so in one embodiment, the binding partner of CD30 is CD30L.

CD43 is known to bind to sialic acid residues, and so in one embodiment, the binding partner of CD43 is sialic acid.

CD44 is known to bind to hyaluronic acid, collagen and oestopontin, and so in one embodiment, the binding partner of CD44 is any of hyaluronic acid, collagen and oestopontin.

CD47 is known to bind to thrombospondin, and so in one embodiment, the binding partner of CD47 is thrombospondin.

CD54 is known to bind to LFA-2, and so in one embodiment, the binding partner of CD54 is LFA-2.

CD58 is known to bind to CD2, and so in one embodiment, the binding partner of CD58 is CD2.

CD62L is known to bind to peripheral lymphonode addressin, and so in one embodiment, the binding partner of CD62L is peripheral lymphonode addressin.

CD95 is known to bind to FasL (CD95L), and so in one embodiment, the binding partner of CD95 is FasL (CD95L).

HLA-DR is known to bind to CD4, and so in one embodiment, the binding partner of HLA-DR is CD4.

As used herein, the term “antibody” includes but is not limited to polyclonal, monoclonal, chimeric, single chain, Fab fragments, fragments produced by a Fab expression library and bispecific antibodies. Such fragments include fragments of whole antibodies which retain their binding activity for a target substance, Fv, F(ab′) and F(ab′)2 fragments, as well as single chain antibodies (scFv), fusion proteins and other synthetic proteins which comprise the antigen-binding site of the antibody. A binding partner comprising only part of an antibody may be advantageous by virtue of optimising the rate of clearance from the blood and may be less likely to undergo non-specific binding due to the Fc part. Also included are domain antibodies (dAbs), diabodies, camelid antibodies and engineered camelid antibodies. Furthermore, for administration to humans, the antibodies and fragments thereof may be humanised antibodies, which are now well known in the art (Janeway et al (2001) Immunobiology., 5th ed., Garland Publishing); An et al (2009) Therapeutic Monoclonal Antibodies: From Bench to Clinic, ISBN: 978-0-470-11791-0).

Also included are asymmetric IgG-like antibodies (eg triomab/quadroma, Trion Pharma/Fresenius Biotech; knobs-into-holes, Genentech; Cross MAbs, Roche; electrostatically matched antibodies, AMGEN; LUZ-Y, Genentech; strand exchange engineered domain (SEED) body, EMD Serono; biolonic, Merus; and Fab-exchanged antibodies, Genmab), symmetric IgG-like antibodies (eg dual targeting (DT)-Ig, GSK/Domantis; two-in-one antibody, Genentech; crosslinked MAbs, karmanos cancer center; mAb², F-star; and Cov X-body, Coy X/Pfizer), IgG fusions (eg dual variable domain (DVD)-Ig, Abbott; IgG-like bispecific antibodies, Eli Lilly; Ts2Ab, Medimmune/AZ; BsAb, ZymoGenetics; HERCULES, Biogen Idec; TvAb, Roche) Fc fusions (eg ScFv/Fc fusions, Academic Institution; SCORPION, Emergent BioSolutions/Trubion, ZymoGenetics/BMS; dual affinity retargeting technology (Fc-DART), MacroGenics; dual (ScFv)₂-Fab, National Research Center for Antibody Medicine) Fab fusions (eg F(ab)₂, Medarex/AMGEN; dual-action or Bis-Fab, Genentech; Dock-and-Lock (DNL), ImmunoMedics; bivalent bispecific, Biotechnol; and Fab-Fv, UCB-Celltech), ScFv- and diabody-based antibodies (eg bispecific T cell engagers (BiTEs), Micromet; tandem diabodies (Tandab), Affimed; DARTs, MacroGenics; Single-chain diabody, Academic; TCR-like antibodies, AIT, Receptor Logics; human serum albumin ScFv fusion, Merrimack; and COMBODIES, Epigen Biotech), IgG/non-IgG fusions (eg immunocytokins, EMDSerono, Philogen, ImmunGene, ImmunoMedics; superantigen fusion protein, Active Biotech; and immune mobilising mTCR Against Cancer, ImmTAC) and oligoclonal antibodies (eg Symphogen and Merus).

The antibody may possess any of the antibody-like scaffolds described by Carter (2006) “Potent antibody therapeutics by design”, Nat Rev Immunol. 6(5): 343-57, and Carter (2011) “Introduction to current and future protein therapeutics: a protein engineering perspective”, Exp Cell Res. 317(9): 1261-9. incorporated herein by reference, together with the specificity determining regions described herein. Thus, the term “antibody” also includes affibodies and non-immunoglobulin based frameworks. Examples include adnectins, anticalins, affilins, trans-bodies, darpins, trimerX, microproteins, fynomers, avimers, centgrins and kalbitor (ecallantide).

The advantages of using antibody fragments, rather than whole antibodies, are several-fold. The smaller size of the fragments may lead to improved pharmacological properties, such as better penetration of solid tissue. Moreover, antigen-binding fragments such as Fab, Fv, ScFv and dAb antibody fragments can be expressed in and secreted from E. coli or yeast, thus allowing convenient production in the laboratory and economical production on a commercial scale. Also, such fragments allow for increased toxological safety because of the lack of the Fc component.

The antibody may be of any of the IgG, IgE, IgA, IgM and IgD classes and may be derived from any species. If the antibody is an IgG, it may be any of IgG1, IgG2, IgG3 or IgG4. It is preferred, however, that when the agent is for administration to a particular host, that the antibody, or at least the constant regions thereof, are derived from that host. For example, when the agent is to be administered to a human, the antibody is preferably a human antibody or a humanized antibody, and so on.

Suitable antibodies that bind to CD70 or CD74 can be made by the skilled person using technology long-established in the art. Methods of preparation of monoclonal antibodies and antibody fragments are well known in the art and include hybridoma technology (Kohler & Milstein (1975) “Continuous cultures of fused cells secreting antibody of predefined specificity. Nature 256: 495-497); antibody phage display (Winter et al (1994) “Making antibodies by phage display technology.” Annu. Rev. Immunol. 12: 433-455); ribosome display (Schaffitzel et al (1999) “Ribosome display: an in vitro method for selection and evolution of antibodies from libraries.” J. Immunol. Methods 231: 119-135); and iterative colony filter screening (Giovannoni et al (2001) “Isolation of anti-angiogenesis antibodies from a large combinatorial repertoire by colony filter screening.” Nucleic Acids Res. 29: E27). Further, antibodies and antibody fragments suitable for use in the present invention are described, for example, in the following publications: “Monoclonal Hybridoma Antibodies: Techniques and Application”, Hurrell (CRC Press, 1982); “Monoclonal Antibodies: A Manual of Techniques”, H. Zola, CRC Press, 1987, ISBN: 0-84936-476-0; “Antibodies: A Laboratory Manual” 1^{st} Edition, Harlow & Lane, Eds, Cold Spring Harbor Laboratory Press, New York, 1988. ISBN 0-87969-314-2; “Using Antibodies: A Laboratory Manual” 2^{nd} Edition, Harlow & Lane, Eds, Cold Spring Harbor Laboratory Press, New York, 1999. ISBN 0-87969-543-9; and “Handbook of Therapeutic Antibodies” Stefan Dübel, Ed., 1st Edition,—Wiley-VCH, Weinheim, 2007. ISBN: 3-527-31453-9.

### Internalisation and Presentation on Surface of Cell

By “the agent is internalised and the T cell antigen is presented on the surface of the cell in a form that can be recognised by a T cell”, we include the meaning that the agent is taken into the cell (e.g. by endocytosis) and the T cell antigen is subsequently presented on the surface of the cell in a form that allows recognition by a T cell. Such recognition can be readily determined by assessing activation of the T cell, for example after contacting the T cell with the cell presenting the T cell antigen and using standard assays for cell proliferation known in the art. Suitable assays for determining the extent of an immune response include ELISpot, intracellular cytokine staining, proliferation assay, activation assays (eg CD69), CD107 mobilisation assays or metabolic assays (eg MTT). Also suitable are assays to detect activation-induced secreted cytokines, for example using ELISA or multiplexed bead technologies.

Internalisation can be assessed using any suitable assay known in the art such as a flow cytometric based assay. For example the agent may be coupled to a fluorochrome such as fluorescein isothiocyanater (FITC) and target cells labelled with the agent. After 1-24 hours, a weak acid (eg citric acid) is added to the target cells and after washing the cells would be analysed on a flow cytometer. The acid will quench the fluorochrome if it is still on the cell surface and there would be no signal on the flow cytometer, thereby demonstrating that the agent has not been internalised. If there is internalisation of the agent, the fluorochrome is not accessible to the acid and there would be no quenching effect. This would mean that there would be a positive signal on the flow cytometer, thereby demonstrating internalisation of the agent.

It will be appreciated that one can determine whether the T cell antigen is being presented to a T cell following internalisation of the T cell antigen rather than by external loading of the antigen onto a cell's surface, using routine procedures. For example, cells may be exposed to the agent of the invention under conditions in which internalisation is prevented. Suitable conditions may be lightly fixing the cells using agents such as paraformaldehyde or glutaraldehyde, or performing the experiments at temperatures around 4° C. or below. If internalisation is a requisite to the T cell antigen being presented, there should be no activation of T cells once internalisation is suppressed. Alternatively, one may use inhibitors of intracellular processing pathways to establish whether presentation of the T cell antigen follows internalisation or is the result of external loading. For example, inhibitors of the MHC Class II intracellular processing pathway may be used as is well known in the art and described further below.

In a preferred embodiment, the T cell antigen is one that is internalised and enters the classical MHC Class II processing pathway. For example, the T cell antigen peptide may be released from the agent by proteolytic degradation in endocytic vesicles and become bound to MHC Class II molecules before being exported to the cell surface. Assessing whether a T cell antigen is processed by the MHC Class II pathway is standard practice in art, and may include testing for processing in the presence and absence of known inhibitors of the pathway such as chloroquinone and monensin.

To facilitate processing of the T cell antigen inside the cell, it is preferred if the T cell antigen is attached to the binding partner for any of CD70, CD74, CD22, HLA-DR, CD23, CD30, CD43, CD44, CD47, CD54, CD55, CD58, CD59, CD62L or CD95 such that the T cell antigen can be released from the binding partner within the cell that expresses any of CD70, CD74, CD22, HLA-DR, CD23, CD30, CD43, CD44, CD47, CD54, CD55, CD58, CD59, CD62L or CD95. Suitable attachments are provided below and include various heterobifunctional crosslinkers such as sulfo-SMCC which can attach peptides to free amine groups, eg on the external surface of an antibody. Other crosslinkers may be used to attach peptides to other functional groups (eg carboxyl, hydroxyl moieties) or to carbohydrate groups. Typically, the attachments are covalent, although strong non-covalent attachments such as Biotin-Avidin or hapten-specific antibodies (eg Digoxigenin) may be used.

In an embodiment, the T cell antigen can be released from the binding partner by an intracellular protease.

Without wishing to be bound by any theory, the inventors believe that, following internalisation of an agent comprising an MHC Class II restricted peptide into the cell, the agent will be processed in the same manner as the MHC Class II processing pathway where the endolysosome would become acidified. Acidification would activate various endosomal and lysosomal based proteases, such as cathepsins, which would together break down the agent and thereby release the peptide from the agent. It will be appreciated that the proteolysis is not acting on the peptide itself. The peptide may then be loaded onto MHC Class II molecules and presented on the cell surface.

Release of the T cell antigen from the binding partner may be tested using T cell antigen specific T cells that would recognise the T cell antigen presented on the cell surface. If the T cell antigen was released, T cells would recognise the T cell antigen on the cell surface and this would be determined by a positive signal in T cell recognition assays as mentioned previously. If the T cell antigen was not released, there would be a negative signal in T cell recognition assays. The T cell antigen may also be labelled using a fluorophore and techniques such as direct cellular imaging used to assess distribution of the T cell antigen.

It will be appreciated that the T cell antigen is not attached to the binding partner of any of CD70, CD74, CD22, HLA-DR, CD23, CD30, CD43, CD44, CD47, CD54, CD55, CD58, CD59, CD62L or CD95 in such a way that the T cell antigen can be released from the binding partner extracellularly. Rather, the T cell antigen must be internalised into the cell expressing any of CD70, CD74, CD22, HLA-DR, CD23, CD30, CD43, CD44, CD47, CD54, CD55, CD58, CD59, CD62L or CD95 and presented on its surface. Preferably, therefore, the T cell antigen is not attached to the binding partner of any of CD70, CD74, CD22, HLA-DR, CD23, CD30, CD43, CD44, CD47, CD54, CD55, CD58, CD59, CD62L or CD95 in a way that the T cell antigen can be released from the binding partner extracellularly, for example by any one or more of an extracellular protease, a nuclease, a lipase, a lyase, a phosphatase or a carbohydrase. To ensure this, it is preferred if the agent does not include a site cleavable by an extracellular molecule (e.g. protease, nuclease, lipase, lyase, phosphatise, carbohydrase) that would act to release the T cell antigen from the binding partner of any of CD70, CD74, CD22, HLA-DR, CD23, CD30, CD43, CD44, CD47, CD54, CD55, CD58, CD59, CD62L or CD95. For example, the agent may not include a site (eg a specific protease cleavage site) cleavable by an extracellular molecule (eg a specific protease) residing in the vicinity of a cell expressing any of CD70, CD74, CD22, HLA-DR, CD23, CD30, CD43, CD44, CD47, CD54, CD55, CD58, CD59, CD62L or CD95, such as a cancer cell. It will be appreciated that this will reduce the necessary size of the agent of the invention. For example, where the T cell antigen is a peptide, the agent of the invention typically comprises (i) a peptide having a length less than 22 amino acids (e.g. less than 21, 20, 19, 18, 17, 16, 15, 14, 13, 12, 11 or 10 amino acids) which peptide comprises or consists of the T cell antigen, and (ii) a binding partner (e.g. antibody) that is attached to the peptide.

Determining whether a given sequence can be cleaved by a protease, and if so which protease, is routine practice for the skilled person. There has been a lot of research into proteolytic cleavage sequences, with many programs available to determine the proteolytic activity towards a given sequence (eg Sigma Aldrich programs). There are also databases (eg MEROPs and PMAP) that contain a wealth of information about proteolysis and protease recognition sequences. Any suitable method may be used.

### Synthesis of Agent of Invention

Conveniently, the T cell antigen is joined to the binding partner of any of CD70, CD74, CD22, HLA-DR, CD23, CD30, CD43, CD44, CD47, CD54, CD55, CD58, CD59, CD62L or CD95 by a linker. By ‘linker’ we include the meaning of a chemical moiety that attaches the binding partner to the T cell antigen.

In an embodiment, the linker does not include a site cleavable by an extracellular molecule, such as an extracellular protease. Thus, in an embodiment any moiety that joins the binding partner to the T cell antigen does not include a site cleavable by an extracellular molecule such as an extracellular protease.

It is appreciated that the T cell antigen may either be bound covalently or non-covalently to the binding partner of any of CD70, CD74, CD22, HLA-DR, CD23, CD30, CD43, CD44, CD47, CD54, CD55, CD58, CD59, CD62L or CD95. Preferably, the T cell antigen is covalently attached to the binding partner of any of CD70, CD74, CD22, HLA-DR, CD23, CD30, CD43, CD44, CD47, CD54, CD55, CD58, CD59, CD62L or CD95.

In one embodiment, the T cell antigen and binding partner of any of CD70, CD74, CD22, HLA-DR, CD23, CD30, CD43, CD44, CD47, CD54, CD55, CD58, CD59, CD62L or CD95, are covalently attached by a linker.

Thus, the T cell antigen (e.g. peptide) and binding partner of any of CD70, CD74, CD22, HLA-DR, CD23, CD30, CD43, CD44, CD47, CD54, CD55, CD58, CD59, CD62L or CD95, may be conveniently linked by any of the conventional ways of cross-linking molecules, such as those generally described in O'Sullivan et al Anal. Biochem. (1979) 100, 100-108. For example, one of the T cell antigen (e.g. peptide) or binding partner of CD70 or CD74 may be enriched with thiol groups and the other reacted with a bifunctional agent capable of reacting with those thiol groups, for example the N-hydroxysuccinimide ester of iodoacetic acid (NHIA) or N-succinimidyl-3-(2-pyridyldithio)propionate (SPDP), a heterobifunctional cross-linking agent which incorporates a disulphide bridge between the conjugated species. Amide and thioether bonds, for example achieved with m-maleimidobenzoyl-N-hydroxysuccinimide ester, are generally more stable in vivo than disulphide bonds.

It is known that bis-maleimide reagents allow the attachment of a thiol group (e.g. thiol group of a cysteine residue of an antibody) to another thiol-containing moiety (e.g. thiol group of a T cell antigen or a linker intermediate), in a sequential or concurrent fashion. Other functional groups besides maleimide, which are reactive with a thiol group include iodoacetamide, bromoacetamide, vinyl pyridine, disulfide, pyridyl disulfide, isocyanate, and isothiocyanate.

Further useful cross-linking agents include S-acetylthioglycolic acid N-hydroxysuccinimide ester (SATA) which is a thiolating reagent for primary amines which allows deprotection of the sulphydryl group under mild conditions (Julian et al (1983) Anal. Biochem. 132, 68), dimethylsuberimidate dihydrochloride and N,N′-o-phenylenedimaleimide.

Particularly preferred crosslinking agents include sulfosuccinimidyl 4-[N-maleimidomethyl]cyclohexane-1-carboxylate (Sulfo-SMCC), sulfosuccinimidyl 6-(3′-[2-pyridyldithio]-propionamido) hexanoate (Sulfo-LC-SPDP) and N-[β-Maleimidopropionic acid] hydrazide, trifluoroacetic acid salt (BMPH).

It will be understood that a large number of homobifunctional and heterobifunctional crosslinking chemistries would be appropriate to join the binding partner of any of CD70, CD74, CD22, HLA-DR, CD23, CD30, CD43, CD44, CD47, CD54, CD55, CD58, CD59, CD62L or CD95 to the T cell antigen, and any such chemistry may be used. For example, Click Chemistry using Staudinger Ligation Chemistry (phosphine-azido chemistry) may be used.

It is appreciated that the T cell antigen and binding partner of any of CD70, CD74, CD22, HLA-DR, CD23, CD30, CD43, CD44, CD47, CD54, CD55, CD58, CD59, CD62L or CD95 do not need to be cross-linked directly to each other, but may be attached via one or more spacer moieties. For example, the T cell antigen may be crosslinked to a chemical moiety which in turn is crosslinked to the binding partner of any of CD70, CD74, CD22, HLA-DR, CD23, CD30, CD43, CD44, CD47, CD54, CD55, CD58, CD59, CD62L or CD95. Generally, a spacer moiety may serve to prevent steric hindrance; however, since the agent is expected to be broken down intracellularly such that the T cell antigen is released, it will be understood that one or more spacer moieties are not required.

In a specific embodiment where the T cell antigen and binding partner of any of CD70, CD74, CD22, HLA-DR, CD23, CD30, CD43, CD44, CD47, CD54, CD55, CD58, CD59, CD62L or CD95 are covalently attached and where both the antigen and binding partner are peptides or polypeptides, it is appreciated that the two components may be part of a fusion polypeptide that may be encoded by a nucleic acid molecule. The invention includes such a nucleic acid molecule and host cells containing them. For example, an antibody binding partner may be genetically engineered to contain the T cell antigen using genetic engineering techniques well established in the art. Thus, it will be appreciated that the T cell antigen may be embedded within, or at the termini of, the polypeptide sequence of the binding partner of any of CD70, CD74, CD22, HLA-DR, CD23, CD30, CD43, CD44, CD47, CD54, CD55, CD58, CD59, CD62L or CD95, provided that it can be released so as to be capable of being presented on the surface of a cell that expresses any of CD70, CD74, CD22, HLA-DR, CD23, CD30, CD43, CD44, CD47, CD54, CD55, CD58, CD59, CD62L or CD95 following internalisation. Suitably, the T cell antigen and the binding partner of any of CD70, CD74, CD22, HLA-DR, CD23, CD30, CD43, CD44, CD47, CD54, CD55, CD58, CD59, CD62L or CD95 are joined so that both portions retain their respective activities such that the agent may be targeted to a cell expressing any of CD70, CD74, CD22, HLA-DR, CD23, CD30, CD43, CD44, CD47, CD54, CD55, CD58, CD59, CD62L or CD95 and the T cell antigen may be presented by the cell so as to elicit an immune response. The T cell antigen and binding partner may be joined by a linker peptide. Suitable linker peptides are those that typically adopt a random coil conformation, for example the polypeptide may contain alanine or proline or a mixture of alanine plus proline residues. Preferably, the linker contains between 2 and 100 amino acid residues, more preferably between 2 and 50 and still more preferably between 4 and 20. However, as discussed above, it will be realised that a linker peptide is not essential given that the agent is broken down intracellularly such that the T cell antigen is released.

Polynucleotides which encode suitable binding partners of any of CD70, CD74, CD22, HLA-DR, CD23, CD30, CD43, CD44, CD47, CD54, CD55, CD58, CD59, CD62L or CD95 are known in the art or can be readily designed from known sequences such as from sequences of proteins known to interact with any of CD70, CD74, CD22, HLA-DR, CD23, CD30, CD43, CD44, CD47, CD54, CD55, CD58, CD59, CD62L or CD95 or contained in nucleotide sequence databases such as the GenBank, EMBL and dbEST databases. Polynucleotides which encode suitable T cell antigens are known in the art or can readily be designed from known sequences and made.

Polynucleotides which encode suitable linker peptides can readily be designed from linker peptide sequences and made.

Thus, polynucleotides which encode the agents used in the invention can readily be constructed using well known genetic engineering techniques.

The nucleic acid is then expressed in a suitable host to produce an agent of the invention. Thus, the nucleic acid encoding the agent of the invention may be used in accordance with known techniques, appropriately modified in view of the teachings contained herein, to construct an expression vector, which is then used to transform an appropriate host cell for the expression and production of the agent of the invention of the invention.

It is appreciated that the nucleic acid encoding the agent of the invention may be joined to a wide variety of other nucleic acid sequences for introduction into an appropriate host. The companion nucleic acid will depend upon the nature of the host, the manner of the introduction of the nucleic acid into the host, and whether episomal maintenance or integration is desired, as is well known in the art.

In an alternative embodiment, the T cell antigen and the binding partner of any of CD70, CD74, CD22, HLA-DR, CD23, CD30, CD43, CD44, CD47, CD54, CD55, CD58, CD59, CD62L or CD95 are non-covalently attached. However, it will be appreciated that the non-covalent attachment must be sufficiently stable to allow the agent to be localised to the cell following administration of the agent to a subject, and to allow the T cell antigen to be presented on its surface. Typically, non-covalent bindings should have an affinity with a K_{d}<10⁻⁹. For non-covalent bindings, immunological bindings or such binding as via biotin/avidin or streptavidin, respectively, are preferred. For example, the binding partner of CD70, CD74, CD22, HLA-DR, CD23, CD30, CD43, CD44, CD47, CD54, CD55, CD58, CD59, CD62L or CD95 may be a bispecific antibody, one specificity of which is directed to an entity expressed by the unwanted cell and one specificity of which is directed to the T cell antigen or part thereof. Also, it is possible to couple the T cell antigen to another substance against which, in turn, the specificity of the bispecific antibody will be directed to. For instance, the T cell antigen may contain further peptidic sequences which are recognised by the bispecific antibody. Another possibility involves coupling the binding partner of any of CD70, CD74, CD22, HLA-DR, CD23, CD30, CD43, CD44, CD47, CD54, CD55, CD58, CD59, CD62L or CD95, for example to streptavidin whilst the T cell antigen is coupled to biotin, and vice versa. Other means by which non-covalent interactions can be formed include leucine zipper sequences or affinity bonds. In any event, the attachment between the T cell antigen and the binding partner of any of CD70, CD74, CD22, HLA-DR, CD23, CD30, CD43, CD44, CD47, CD54, CD55, CD58, CD59, CD62L or CD95 must be such that, following internalisation of the agent into the cell expressing any of CD70, CD74, CD22, HLA-DR, CD23, CD30, CD43, CD44, CD47, CD54, CD55, CD58, CD59, CD62L or CD95 respectively, the T cell antigen can be presented on the surface of the cell in a form that can be recognised by a T cell.

Amino acid residues described herein are generally in the natural “L” isomeric form. However, residues in the “D” isomeric form can be substituted for L-amino acid residues in certain situations, provided that the T cell antigen of the agent can still be presented on the surface of the cell expressing CD70 or CD74 in a form that can be recognised by a T cell. The definition also includes, unless otherwise specifically indicated, chemically-modified amino acids, including amino acid analogues (such as penicillamine, 3-mercapto-D-valine), naturally-occurring non-proteogenic amino acids (such as norleucine), beta-amino acids, azapeptides, N-methylated amino acids and chemically-synthesised compounds that have properties known in the art to be characteristic of an amino acid. The term “proteogenic” indicates that the amino acid can be incorporated into a protein in a cell through well-known metabolic pathways. The definition also includes amino acids in which the functional side group has been chemically derivatised. Such derivatised molecules include, for example, those molecules in which free amino groups have been derivatised to form amine hydrochlorides, p-toluene sulfonyl groups, carbobenzoxy groups, t-butyloxycarbonyl groups, chloroacetyl groups or formyl groups. Free carboxyl groups may be derivatised to form salts, methyl and ethyl esters or other types of esters or hydrazides. Free hydroxyl groups may be derivatised to form O-acyl or O-alkyl derivatives. Also included as derivatives are those peptide portions that contain one or more naturally occurring amino acid derivatives of the twenty standard amino acids.

Accordingly, it is appreciated that the peptide portions of the agent of the invention can be peptide “mimetics”, i.e. peptidomimetics which mimic the structural features of peptides comprising or consisting of the amino acid sequence as described herein. Peptidomimetics can be even more advantageous in therapeutic use, in the resistance to degradation, in permeability or in possible oral administration.

A primary goal in the design of peptide mimetics has been to reduce the susceptibility of mimetics to cleavage and inactivation by peptidases. In one approach, such as disclosed by Sherman et al (1990), one or more amide bonds have been replaced in an essentially isosteric manner by a variety of chemical functional groups. This stepwise approach has met with some success in that active analogues have been obtained. In some instances, these analogues have been shown to possess longer biological half-lives than their naturally-occurring counterparts. In another approach, a variety of uncoded or modified amino acids such as D-amino acids and N-methyl amino acids have been used to modify mammalian peptides. Alternatively, a presumed bioactive conformation has been stabilised by a covalent modification, such as cyclization or by incorporation of γ-lactam or other types of bridges (Veber et al, 1978) and Thorsett et al, 1983). Another approach, disclosed by Rich (1986) has been to design peptide mimics through the application of the transition state analogue concept in enzyme inhibitor design. For example, it is known that the secondary alcohol of statine mimics the tetrahedral transition state of the sessile amide bond of the pepsin substrate. Other approaches include the use of azapeptides and beta-amino acids.

Also included in the definition of ‘peptidomimetics’, are retro-inverso peptides. By retro-inverso peptides (also known as all-D-retro or retro-enantio peptides) we include the meaning of a peptide in which all of the L-amino acids are replaced with D-amino acids and the peptide bonds are reversed. Thus, the peptides are composed of D-amino acids assembled in the reverse order from that of the parent L-sequence. Retro-inverso peptides can be synthesised by methods known in the art, for example such as those described in Meziere et al (1997) J. Immunol. 159 3230-3237. This approach involves making pseudopeptides containing changes involving the backbone, and not the orientation of side chains which remain very similar to the parent peptide. Retro-inverse peptides are much more resistant to proteolysis.

Therefore, it will be appreciated that when any of the binding partner of any of CD70, CD74, CD22, HLA-DR, CD23, CD30, CD43, CD44, CD47, CD54, CD55, CD58, CD59, CD62L or CD95, T cell antigen, and spacer moieties as described herein are peptides or polypeptides, any one or more of those peptides or polypeptides may be substituted for a corresponding peptidomimetic that retains the respective activity of the parent peptide or polypeptide. This may help to confer protease resistance on the agent of the invention and thereby improve its stability. Thus, for example, when a T cell antigen is attached to a binding partner of any of CD70, CD74, CD22, HLA-DR, CD23, CD30, CD43, CD44, CD47, CD54, CD55, CD58, CD59, CD62L or CD95 via one or more peptide spacer moieties, it may be desirable for one or more of those spacer moieties to be peptidomimetics, e.g. wherein one or more of the naturally occurring amino acids of the spacer moieties are replaced or modified, for example, to improve stability.

Another approach to increase stability of peptide portions of the agent of the invention is to have stabilising groups at one or both termini. Typical stabilising groups include amido, acetyl, benzyl, phenyl, tosyl, alkoxycarbonyl, alkyl carbonyl, benzyloxycarbonyl and the like end group modifications. Additional modifications include using a “D” amino acid in place of a “L” amino acid at the termini, and amide rather than amino or carboxy termini or acetyl rather than amino termini, to inhibit exopeptidase activity. Thus, it is appreciated that whenever the agent of the invention has an exposed peptide terminus, that terminus may have a capping moiety, preferably a moiety that is less than 200 Da in molecular weight. Further capping moieties include a naftyl group or a polyethylene glycol group. It is appreciated that retro-inverso peptides are already relatively stable and so may not require additional capping moieties.

Preferably, the agent of the invention has a half-life in plasma of at least 24 hours at 37° C.

It may be desirable to modify the agent of the invention so that it can be more easily detected, for example by biotinylating it or by incorporating any detectable label known in the art such as radiolabels, fluorescent labels or enzymatic labels.

As described above, the inventors have shown that agents of the invention may be used to redirect existing immune responses to kill particular cells expressing any of CD70, CD74, CD22, HLA-DR, CD23, CD30, CD43, CD44, CD47, CD54, CD55, CD58, CD59, CD62L or CD95 in a specific manner. Since cells expressing any of CD70, CD74, CD22, HLA-DR, CD23, CD30, CD43, CD44, CD47, CD54, CD55, CD58, CD59, CD62L or CD95 are often cells which, at least in part, mediate the pathology of a biological or medical condition or disorder, the agents of the invention offer significant therapeutic potential.

Accordingly, a second aspect of the invention provides an agent according to the first aspect of the invention for use in medicine.

A third aspect of the invention also provides a pharmaceutical composition, comprising an agent according to the first aspect of the invention and a pharmaceutically acceptable carrier, diluent or excipient.

A fourth aspect of the invention provides a method of preventing or treating a condition characterised by the presence of cells expressing any of CD70, CD74, CD22, HLA-DR, CD23, CD30, CD43, CD44, CD47, CD54, CD55, CD58, CD59, CD62L or CD95, the method comprising administering an agent according to the first aspect of the invention. In this way, the agent of the invention will bind to the cells expressing any of CD70, CD74, CD22, HLA-DR, CD23, CD30, CD43, CD44, CD47, CD54, CD55, CD58, CD59, CD62L or CD95, and, following internalisation, the T cell antigen will be presented on the surface of the cells making them a target for T cells. For the avoidance of doubt, an agent that comprises a binding partner for a particular target (eg CD70) will be used to prevent or treat a condition characterised by the presence of cells expressing that particular target (eg CD70).

Thus, the method may involve identifying a subject who has a condition or who is at risk of developing a condition characterised by any of CD70, CD74, CD22, HLA-DR, CD23, CD30, CD43, CD44, CD47, CD54, CD55, CD58, CD59, CD62L or CD95 (eg cancer), administering the agent according to the first aspect of the invention to the subject, and monitoring the levels of the cells expressing any of CD70, CD74, CD22, HLA-DR, CD23, CD30, CD43, CD44, CD47, CD54, CD55, CD58, CD59, CD62L or CD95 in the subject either by conducting tests to determine the number of cells expressing any of CD70, CD74, CD22, HLA-DR, CD23, CD30, CD43, CD44, CD47, CD54, CD55, CD58, CD59, CD62L or CD95 or by monitoring the clinical symptoms of the subject. Depending on the results of the monitoring step, it may be necessary to administer more of the agent.

The invention includes an agent according to the first aspect of the invention for use in preventing or treating a condition characterised by the presence of cells expressing any of CD70, CD74, CD22, HLA-DR, CD23, CD30, CD43, CD44, CD47, CD54, CD55, CD58, CD59, CD62L or CD95.

The invention includes the use of an agent according to the first aspect of the invention in the preparation of a medicament for preventing or treating a condition characterised by the presence of cells expressing any of CD70, CD74, CD22, HLA-DR, CD23, CD30, CD43, CD44, CD47, CD54, CD55, CD58, CD59, CD62L or CD95.

By a ‘condition characterised by the presence of cells expressing any of CD70, CD74, CD22, HLA-DR, CD23, CD30, CD43, CD44, CD47, CD54, CD55, CD58, CD59, CD62L or CD95’ we include any biological or medical condition or disorder in which at least part of the pathology is mediated by the presence of cells expressing any of CD70, CD74, CD22, HLA-DR, CD23, CD30, CD43, CD44, CD47, CD54, CD55, CD58, CD59, CD62L or CD95. The condition may be caused by the presence of the cells expressing any of CD70, CD74, CD22, HLA-DR, CD23, CD30, CD43, CD44, CD47, CD54, CD55, CD58, CD59, CD62L or CD95 or else the presence of the cells expressing any of CD70, CD74, CD22, HLA-DR, CD23, CD30, CD43, CD44, CD47, CD54, CD55, CD58, CD59, CD62L or CD95 may be an effect of the condition.

CD70 and CD74 are generally expressed by B cells, and so typically the condition is one where at least part of the pathology is mediated by B cells. For example, CD70 (CD27L) is a member of the tumour necrosis factor family aberrantly expressed on a number of hematologic malignancies and some carcinomas (eg renal cell carcinoma; Jilaveanu et al, Human Palhol 43(9): 1394). CD74 is expressed in parallel with MHC Class-II molecules and so is expressed on professional antigen presenting cells such as B-cells, monocytes, macrophages and dendritic cells. The condition may be one that affects one or more of these cells.

Cells expressing CD70 or CD74 are frequently implicated in lymphoma and several types of carcinoma. Thus, it is particularly preferred if the condition is a tumour (eg a malignant disease) and the cells expressing CD70 or CD74 are tumour cells or tumour associated tissue (eg tumour fibroblasts or tumour blood vessels). The condition may be any cancer such as breast cancer, ovarian cancer, endometrial cancer, cervical cancer, bladder cancer, renal cancer, melanoma, lung cancer, prostate cancer, testicular cancer, thyroid cancer, brain cancer, oesophageal cancer, gastric cancer, pancreatic cancer, colorectal cancer, liver cancer, leukaemia, myeloma, non-Hodgkin's lymphoma, Hodgkin's lymphoma, acute myeloid leukaemia, acute lymphoblastic leukaemia, chronic lymphoblastic leukaemia, lymphoproliferative disorder, myelodysplastic disorder, myeloproliferative disease and premalignant disease.

Cells expressing CD74 have also been associated with allergic and autoimmune disease, and so in a further preferred embodiment when the binding partner is for CD74, the condition is allergic or autoimmune disease. Examples include rheumatoid arthritis, systemic lupus erythematosus and immune thrombocytopenia purpura.

Conditions characterised by the presence of cells expressing any of the other targets disclosed herein, include those mentioned in the above discussion of those targets.

The table below provides cellular expression data for each of the targets disclosed herein and conditions associated with each target. Thus, when the agent of the invention comprises a binding partner for a particular target, the agent may be used to prevent or treat one of the conditions associated with that target as set out in the table below.

| | | Diseases associated with |
|---|---|---|
| Target | Cellular Expression Data | Target |
| HLA-DR | B-cells | B-cell Lymphomas (e.g. |
| | | Hodgkin Lymphoma, Non- |
| | | Hodgkin Lymphoma, B-cell |
| | | chronic lymphocytic |
| | | leukaemia) |
| | B-cells | Autoimmune diseases (e.g. |
| | | rheumatoid arthritis, systemic |
| | | lupus erythematosus, |
| | | autoimmune cytopenias) |
| | Myeloid Cells (Macrophages, | Myeloid Leukaemias |
| | Kupfer cells) | |
| | Antigen Presenting cells | |
| | (Dendritic Cells, Langerhans | |
| | cells) | |
| | Plasma Cells | Myeloma, Amyloid, |
| | | Plasmacytomas |
| | | Autoimmune diseases (e.g. |
| | | rheumatoid arthritis, systemic |
| | | lupus erythematosus, |
| | | autoimmune cytopenias) |
| CD74 | B-cells | B-cell Lymphomas (e.g. |
| | | Hodgkin Lymphoma, Non- |
| | | Hodgkin Lymphoma) |
| | B-cells | Autoimmune diseases (e.g. |
| | | rheumatoid arthritis, systemic |
| | | lupus erythematosus, |
| | | autoimmune cytopenias) |
| | Myeloid Cells (Macrophages, | Myeloid Leukaemias |
| | Kupfer cells) | |
| | Antigen Presenting cells | |
| | (Dendritic Cells, Langerhans | |
| | cells) | |
| | Plasma Cells | Myeloma, Amyloid, |
| | | Plasmacytomas |
| | | Autoimmune diseases (e.g. |
| | | rheumatoid arthritis, systemic |
| | | lupus erythematosus, |
| | | autoimmune cytopenias) |
| CD22 | B-cells | B-cell Lymphomas (e.g. |
| | | Hodgkin Lymphoma, Non- |
| | | Hodgkin Lymphoma, B-cell |
| | | chronic lymphocytic |
| | | leukaemia) |
| | B-cells | Autoimmune diseases (e.g. |
| | | rheumatoid arthritis, systemic |
| | | lupus erythematosus, |
| | | autoimmune cytopenias) |
| CD23 | B-cells | B-cell Lymphomas (e.g. |
| | | Hodgkin Lymphoma, Non- |
| | | Hodgkin Lymphoma, B-cell |
| | | chronic lymphocytic |
| | | leukaemia) |
| | B-cells | Autoimmune diseases (e.g. |
| | | rheumatoid arthritis, systemic |
| | | lupus erythematosus, |
| | | autoimmune cytopenias) |
| CD43 | T-cells | Autoimmune diseases (e.g. |
| | | diabetes melitus, |
| | | anutoimmune hepatitis). |
| | Monocytes | |
| | B-cells | B-cell lymphoblastic |
| | | lymphoma, Mucosa |
| | | associated lymphoid tissue |
| | | lymphoma |
| CD44 | Cancer Stem Cells | Many types of cancer |
| | | including Breast, Colorectal, |
| | | Ovarian, Head and Neck, |
| | | leukaemias and |
| | | gastrointestinal carcinomas. |
| | Squamous Cell Carcinoma | Head and Neck Cancers |
| CD47 | Wide tissue expression | |
| CD54 | Vacular Endothelium | Vasculitis, Kaposi's sarcoma |
| | T-cells | Autoimmune diseases (e.g. |
| | | diabetes melitus, |
| | | anutoimmune hepatitis). |
| | B-cells | B-cell lymphoblastic |
| | | lymphoma, Mucosa |
| | | associated lymphoid tissue |
| | | lymphoma |
| CD58 | Vacular Endothelium | Vasculitis, Kaposi's sarcoma |
| | T-cells | Autoimmune diseases (e.g. |
| | | diabetes melitus, |
| | | anutoimmune hepatitis). |
| | B-cells | B-cell lymphoblastic |
| | | lymphoma, Mucosa |
| | | associated lymphoid tissue |
| | | lymphoma |
| CD55 | Widespread Tissue | |
| | expression (Blood and | |
| | Epithelia) | |
| CD59 | Widespread Tissue | |
| | expression (Blood and | |
| | Epithelia) | |
| CD62L | B-cells | Chronic lymphocytic |
| | | leukaemia, |
| | T-cells | Adult T cell leukaemia |
| CD95 | Ubiquitous - can be | Many including ovarian, liver |
| | upregulated on many cell | and colorectal carcinoma, |
| | types | |
| CD30 | Activated T cells | Anaplastic large cell |
| | | lymphoma & embryonal |
| | | carcinoma |
| | Activated B cells | Classical Hodgkin |
| | | lymphoma |
| CD70 | B Cells | Hodgkin lymphoma, Non- |
| | | Hodgkin lymphoma |
| | Kidney | Renal cell carcinoma |
| | | Also pancreatic (25%), |
| | | larynx/pharynx (22%), |
| | | melanoma (16%), ovarian |
| | | (15%), lung (10%), and colon |
| | | (9%) |

Other conditions characterised by the presence of cells expressing any of CD70, CD74, CD22, HLA-DR, CD23, CD30, CD43, CD44, CD47, CD54, CD55, CD58, CD59, CD62L or CD95 can be readily determined by the skilled person. For example, the expression profile of any of CD70, CD74, CD22, HLA-DR, CD23, CD30, CD43, CD44, CD47, CD54, CD55, CD58, CD59, CD62L or CD95 on cells may be carried out on a biopsy sample (eg from a cancer patient) using routine assays for measuring nucleic acid (e.g. DNA or RNA transcripts) or protein levels. Transcriptomic or proteomic techniques may be used. Also, immunohistochemistry and immunofluorescence may be used to quantitate antigen expression in tissues.

By preventing or treating a condition we include the meaning of reducing or alleviating symptoms in a patient (i.e. palliative use), preventing symptoms from worsening or progressing, treating the disorder (e.g. by inhibition or elimination of the causative agent), or prevention of the condition or disorder in a subject who is free therefrom.

It will be appreciated that the agents of the invention lend themselves to personalised medicine in the clinic whereby the most appropriate agent to be administered to the patient is determined, and either selected or prepared in the clinic. For example, before the step of administering the agent to the subject, any of the following may be determined: (i) the MHC alleles of the subject and/or (ii) the T cell response (eg cytotoxic T cells response) of the subject to a T cell antigen. The MHC alleles of a subject can be assessed by serological assays at the antigen level or by using DNA assays at the genetic level. Determining whether a given antigen stimulates a specific T cell response (eg cytotoxic T cell response) in a subject can be done by contacting isolated peripheral mononuclear blood cells from the subject with the antigen and using standard assays for cell proliferation.

Thus the method of the fourth aspect of the invention may include the steps of (i) identifying a subject who has a condition, or who is at risk of developing a condition characterised by the presence of cells expressing any of CD70, CD74, CD22, HLA-DR, CD23, CD30, CD43, CD44, CD47, CD54, CD55, CD58, CD59, CD62L or CD95 (eg cancer), (ii) taking a sample from the subject, (iii) analysing the sample to identify the optimum T cell antigen preventing or treating the condition in that subject, (iii) preparing the agent of the invention, (iv) administering the agent to the subject, and (v) monitoring the levels of cells expressing any of CD70, CD74, CD22, HLA-DR, CD23, CD30, CD43, CD44, CD47, CD54, CD55, CD58, CD59, CD62L or CD95 in the subject either by conducting tests to determine the number of cells expressing any of CD70, CD74, CD22, HLA-DR, CD23, CD30, CD43, CD44, CD47, CD54, CD55, CD58, CD59, CD62L or CD95 or by monitoring the clinical symptoms of the subject.

It is appreciated that an apparatus may be used to select and optionally prepare the most appropriate agent to be used for a particular patient. For example, the apparatus may perform an automated analysis of one or more samples from the subject, and based on this analysis select and optionally prepare a tailor-made agent for that subject. Thus the apparatus may perform serological assays on the sample to determine a subject's MHC alleles and based on this test various peptides for their efficiency in eliciting a T cell response (eg cytotoxic T cell response), so as to identify the best T cell antigen for use in that patient. Similarly, the apparatus may carry out an expression profile of cells from the subject (eg from a biopsy sample) so as to determine a suitable binding partner for any of CD70, CD74, CD22, HLA-DR, CD23, CD30, CD43, CD44, CD47, CD54, CD55, CD58, CD59, CD62L or CD95.

By performing any one or more of these steps in the clinic an agent tailored for a particular subject can be prepared. For example, the agent can contain a T cell antigen that is known to bind to patient's MHC molecules and elicit a strong T cell response.

In one embodiment, the subject is administered a further therapeutic agent in addition to the agent according to the first aspect of the invention. For example, when administering the agent to prevent or treat a particular condition, a further therapeutic agent known to be useful for combating that condition may be administered. As an example, when the agent is for treating cancer, a further anti-cancer agent (eg anti-neoplastic chemotherapy) may be administered to the subject alongside the agent of the invention. Similarly, the further therapeutic agent may be one that is known to have therapeutic application in allergic disease, inflammatory disease, regenerative medicine and neuroregenerative disease.

It is appreciated that the further therapeutic agent may be administered at the same time as the agent of the invention (i.e. simultaneous administration optionally in a co-formulation) or at a different time to the agent of the invention (i.e. sequential administration).

The further therapeutic agent may be any one or more of a vaccine; an immuno stimulatory drug; an anti-cancer agent; an agent inhibiting an antibody response against the agent of the invention; and/or a protease inhibitor.

For example, in order to boost the effector immune response against the particular T cell antigen used, it may be desirable to vaccinate the subject with the T cell antigen; and/or administer immunostimulating agents such as IL-2, IL-7, IFNα, GM-CSF, metformin, lenalidomide; and/or administer anti-immunoregulatory agents such as Ipilimumab; all of which may be considered as further therapeutic agents. Similarly, the further therapeutic agent may be a live virus such as CMV that is used to stimulate an immune response against the T cell antigen. This may be performed by blood transfusion for example.

It is also appreciated that if the subject is one to whom is administered immunosuppressive agents, that these immunosuppressive agents are withdrawn from the subject (e.g. by suspending treatment) when or before being administered the agent of the invention.

Similarly, it may be desirable to employ methods aimed at circumventing any immunogenicity issues relating to the agent of the invention whereby an adverse antibody response is elicited in vivo. For example, the subject may also be administered one or more agents that are known to inhibit the activity of B cells, such as any of Rituximab, cyclophosphamide, Syk inhibitors, an anti-BAFF antibody (eg Belimumab), an anti-CD22 antibody, an anti-CD20 antibody and an anti-CD19 antibody, all of which may be considered as further therapeutic agents. In this case, it is particularly preferred if the inhibitor of B cells is administered to the subject prior to the agent of the invention, eg as a pre-treatment to ablate B cells.

The invention thus includes a composition comprising (i) an agent according to the first aspect of the invention and (ii) a further therapeutic agent, for use in preventing or treating a condition characterised by the presence of cells expressing any of CD70, CD74, CD22, HLA-DR, CD23, CD30, CD43, CD44, CD47, CD54, CD55, CD58, CD59, CD62L or CD95. Given that the agent of the invention and the further therapeutic agent may be administered simultaneously or sequentially, it will be appreciated that the invention includes an agent according to the first aspect of the invention for use in preventing or treating a condition characterised by the presence of cells expressing any of CD70, CD74, CD22, HLA-DR, CD23, CD30, CD43, CD44, CD47, CD54, CD55, CD58, CD59, CD62L or CD95 in a subject who is administered a further therapeutic agent. It also follows that the invention includes a therapeutic agent for use in preventing or treating a condition characterised by the presence of cells expressing any of CD70, CD74, CD22, HLA-DR, CD23, CD30, CD43, CD44, CD47, CD54, CD55, CD58, CD59, CD62L or CD95 in a subject who is administered an agent according to the first aspect of the invention.

Similarly, the invention includes a use of a composition comprising (i) an agent according to the first aspect of the invention and (ii) a further therapeutic agent, in the manufacture of a medicament for preventing or treating a condition characterised by the presence of cells expressing any of CD70, CD74, CD22, HLA-DR, CD23, CD30, CD43, CD44, CD47, CD54, CD55, CD58, CD59, CD62L or CD95. Again, given that the agent of the invention and the further therapeutic agent may be administered simultaneously or sequentially, it will be appreciated that the invention includes the use of a composition comprising an agent according to the first aspect of the invention in the manufacture of a medicament for preventing or treating a condition characterised by the presence of cells expressing any of CD70, CD74, CD22, HLA-DR, CD23, CD30, CD43, CD44, CD47, CD54, CD55, CD58, CD59, CD62L or CD95 in a subject who is administered a further therapeutic agent. It also follows that the invention includes the use of a therapeutic agent in the manufacture of a medicament for preventing or treating a condition characterised by the presence of cells expressing any of CD70, CD74, CD22, HLA-DR, CD23, CD30, CD43, CD44, CD47, CD54, CD55, CD58, CD59, CD62L or CD95 in a subject who is administered an agent according to the first aspect of the invention.

The invention also provides a composition comprising (i) an agent according to the first aspect of the invention and (ii) a further therapeutic agent suitable for preventing or treating the same condition characterised by the presence of cells expressing any of CD70, CD74, CD22, HLA-DR, CD23, CD30, CD43, CD44, CD47, CD54, CD55, CD58, CD59, CD62L or CD95. It is appreciated that the therapeutic agent mentioned in the immediately preceding two paragraphs may be agents suitable for treating the same condition characterised by the presence of unwanted cells, as treatable by the agents of the invention.

Whilst it is possible for the agent of the invention to be administered alone, it is preferable to present it as a pharmaceutical formulation, together with one or more acceptable carriers. The carrier(s) must be “acceptable” in the sense of being compatible with the therapeutic agent and not deleterious to the recipients thereof. Typically, the carriers will be water or saline which will be sterile and pyrogen free.

Where appropriate, the formulations may conveniently be presented in unit dosage form and may be prepared by any of the methods well known in the art of pharmacy. Such methods include the step of bringing into association the active ingredient (agent for treating or preventing a condition characterised by unwanted cells) with the carrier which constitutes one or more accessory ingredients. In general, the formulations are prepared by uniformly and intimately bringing into association the active ingredient with liquid carriers or finely divided solid carriers or both, and then, if necessary, shaping the product.

Formulations in accordance with the present invention suitable for oral administration may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. povidone, gelatin, hydroxypropylmethyl cellulose), lubricant, inert diluent, preservative, disintegrant (e.g. sodium starch glycolate, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

The agent of the invention can be administered in the form of a suppository or pessary, or they may be applied topically in the form of a lotion, solution, cream, ointment or dusting powder. The agent may also be transdermally administered, for example, by the use of a skin patch.

Preferred unit dosage formulations are those containing a daily dose or unit, daily sub-dose or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavouring agents.

The amount of the agent which is administered to the individual is an amount effective to combat the particular individual's condition. The amount may be determined by the physician.

Preferably, in the context of any aspect of the invention described herein, the subject to be treated is a human. Alternatively, the subject may be an animal, for example a domesticated animal (for example a dog or cat), laboratory animal (for example laboratory rodent, for example mouse, rat or rabbit) or an animal important in agriculture (i.e. livestock), for example horses, cattle, sheep or goats.

In a preferred embodiment of the invention, the T cell antigen in the agent is a peptide, and the agent is used to prevent or treat cancer.

In an embodiment, the binding partner of CD70 is an anti-CD70 antibody such as BU69 antibody (anti-CD70, Birmingham University) (Leucocyte Typing V (1995): edited by SF Schlossman, OUP, Oxford) and the T cell antigen comprises DYSNTHSTRYV (SEQ ID No: 9) or PDDYSNTHSTRYV (SEQ ID No: 108).

In an embodiment, the binding partner of CD74 is an anti-CD74 antibody such as anti-CD74 (eBiosciences) anti-human CD74 purified, Clone: LN2) and the T cell antigen comprises DYSNTHSTRYV (SEQ ID No: 9) or PDDYSNTHSTRYV (SEQ ID No: 108).

In an embodiment, the binding partner of CD70 is an anti-CD70 antibody such as BU69 antibody (anti-CD70, Birmingham University) (Leucocyte Typing V (1995): edited by SF Schlossman, OUP, Oxford) and the T cell antigen comprises PRSPTVFYNIPPMPLPPSQL (SEQ ID No: 49).

In an embodiment, the binding partner of CD22 is an anti-CD22 antibody such as anti-CD22 (BD Bioscience) anti-human CD22 purified, Clone: HIB22) and the T cell antigen comprises DYSNTHSTRYV (SEQ ID No: 9) or PDDYSNTHSTRYV (SEQ ID No: 108).

In an embodiment, the binding partner of CD23 is an anti-CD23 antibody such as anti-CD23 (BD Bioscience) anti-human CD23 purified, Clone: M-L233) and the T cell antigen comprises DYSNTHSTRYV (SEQ ID No: 9) or PDDYSNTHSTRYV (SEQ ID No: 108).

In an embodiment, the binding partner of CD30 is an anti-CD30 antibody such as anti-CD30 (BD Bioscience) anti-human CD30 purified, Clone: BerH8) and the T cell antigen comprises DYSNTHSTRYV (SEQ ID No: 9) or PDDYSNTHSTRYV (SEQ ID No: 108).

In an embodiment, the binding partner of CD43 is an anti-CD43 antibody such as anti-CD43 (BD Bioscience) anti-human CD43 purified, Clone: 1G10) and the T cell antigen comprises DYSNTHSTRYV (SEQ ID No: 9) or PDDYSNTHSTRYV (SEQ ID No: 108).

In an embodiment, the binding partner of CD44 is an anti-CD44 antibody such as anti-CD44 (BD Bioscience) anti-human CD44 purified, Clone: 515) and the T cell antigen comprises DYSNTHSTRYV (SEQ ID No: 9) or PDDYSNTHSTRYV (SEQ ID No: 108).

In an embodiment, the binding partner of CD47 is an anti-CD47 antibody such as anti-CD47 (BD Bioscience) anti-human CD47 purified, Clone: B6H12) and the T cell antigen comprises DYSNTHSTRYV (SEQ ID No: 9) or PDDYSNTHSTRYV (SEQ ID No: 108).

In an embodiment, the binding partner of CD54 is an anti-CD54 antibody such as anti-CD54 (BD Bioscience) anti-human CD54 purified, Clone: 28/CD54) and the T cell antigen comprises DYSNTHSTRYV (SEQ ID No: 9) or PDDYSNTHSTRYV (SEQ ID No: 108).

In an embodiment, the binding partner of CD55 is an anti-CD55 antibody such as anti-CD55 (BD Bioscience) anti-human CD55 purified, Clone: IA10) and the T cell antigen comprises DYSNTHSTRYV (SEQ ID No: 9) or PDDYSNTHSTRYV (SEQ ID No: 108).

In an embodiment, the binding partner of CD58 is an anti-CD58 antibody such as anti-CD58 (BD Bioscience) anti-human CD58 purified, Clone: 1C3 (AICD58.6)) and the T cell antigen comprises DYSNTHSTRYV (SEQ ID No: 9) or PDDYSNTHSTRYV (SEQ ID No: 108).

In an embodiment, the binding partner of CD59 is an anti-CD59 antibody such as anti-CD59 (BD Bioscience) anti-human CD59 purified, Clone: p282 (H19)) and the T cell antigen comprises DYSNTHSTRYV (SEQ ID No: 9) or PDDYSNTHSTRYV (SEQ ID No: 108).

In an embodiment, the binding partner of CD62L is an anti-CD62L antibody such as anti-CD62L (BD Bioscience) anti-human CD62L purified, Clone: SK11) and the T cell antigen comprises DYSNTHSTRYV (SEQ ID No: 9) or PDDYSNTHSTRYV (SEQ ID No: 108).

In an embodiment, the binding partner of CD95 is an anti-CD95 antibody such as anti-CD95 (BD Bioscience) anti-human CD95 purified, Clone: EOS9.1) and the T cell antigen comprises DYSNTHSTRYV (SEQ ID No: 9) or PDDYSNTHSTRYV (SEQ ID No: 108).

In an embodiment, the binding partner of HLA-DR is an anti-HLA-DR antibody such as anti-HLA-DR (BD Bioscience) anti-human HLA-DR purified, Clone: G46-6) and the T cell antigen comprises DYSNTHSTRYV (SEQ ID No: 9) or PDDYSNTHSTRYV (SEQ ID No: 108).

The invention will be described in further detail with the aid of the following Figures and Examples.

FIG. 1: Amino acid sequences of human CD70, CD74, HLA-DR, CD22, CD23, CD30, CD43, CD44, CD47, CD54, CD55, CD58, CD59, CD62L or CD95 (SEQ ID Nos: 93-107).

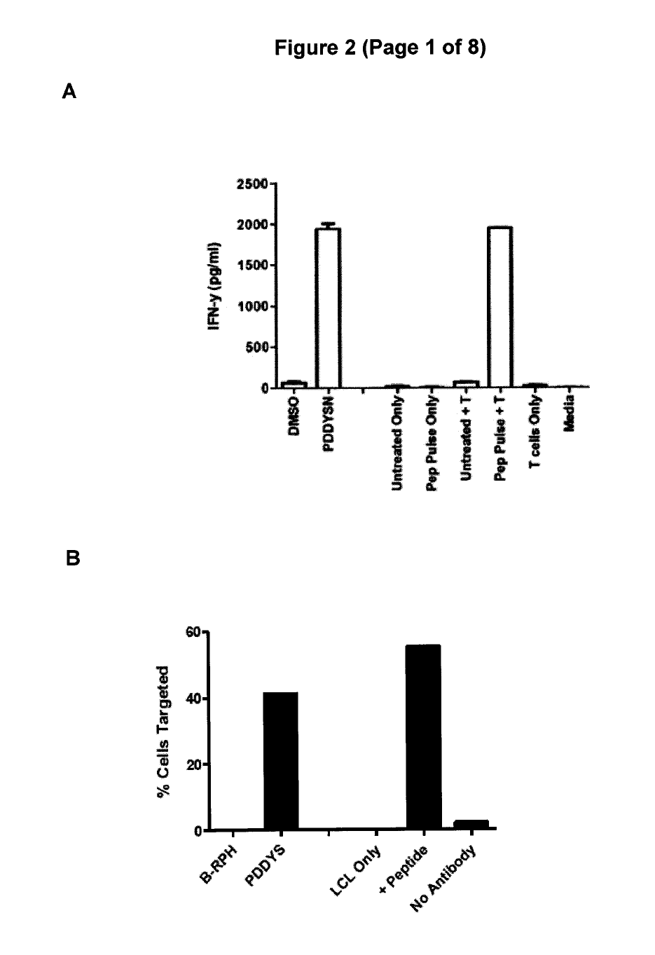

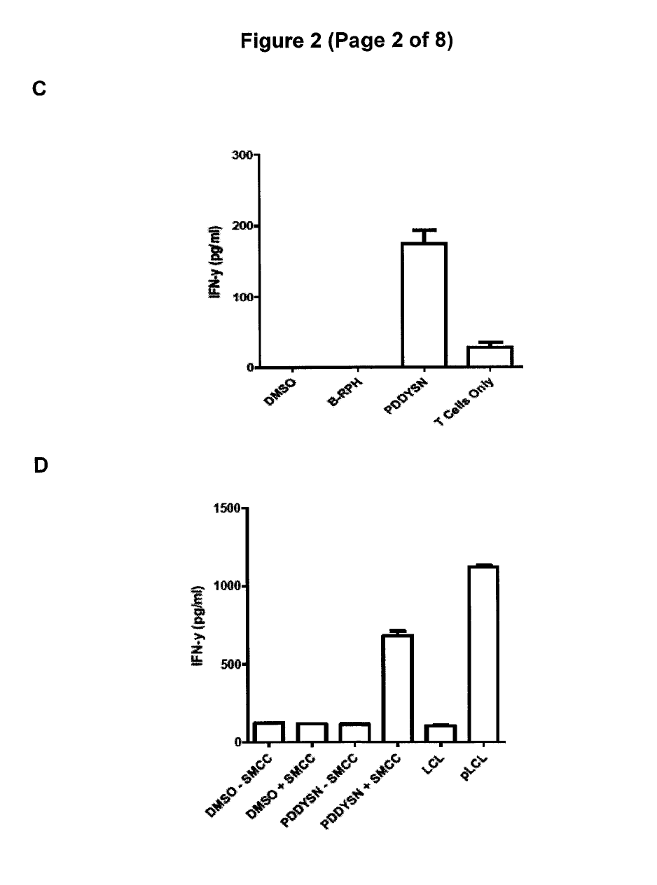

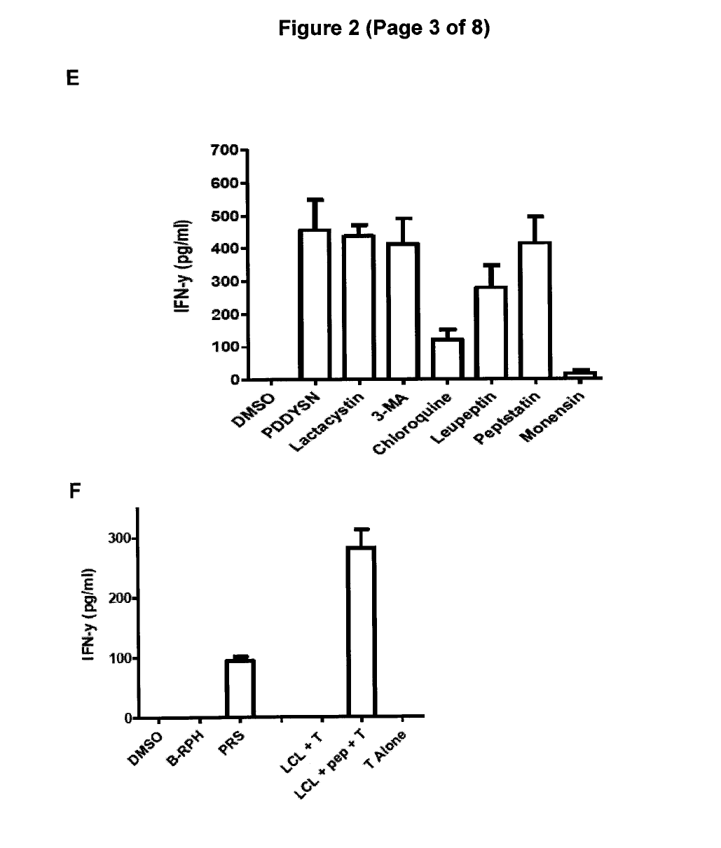

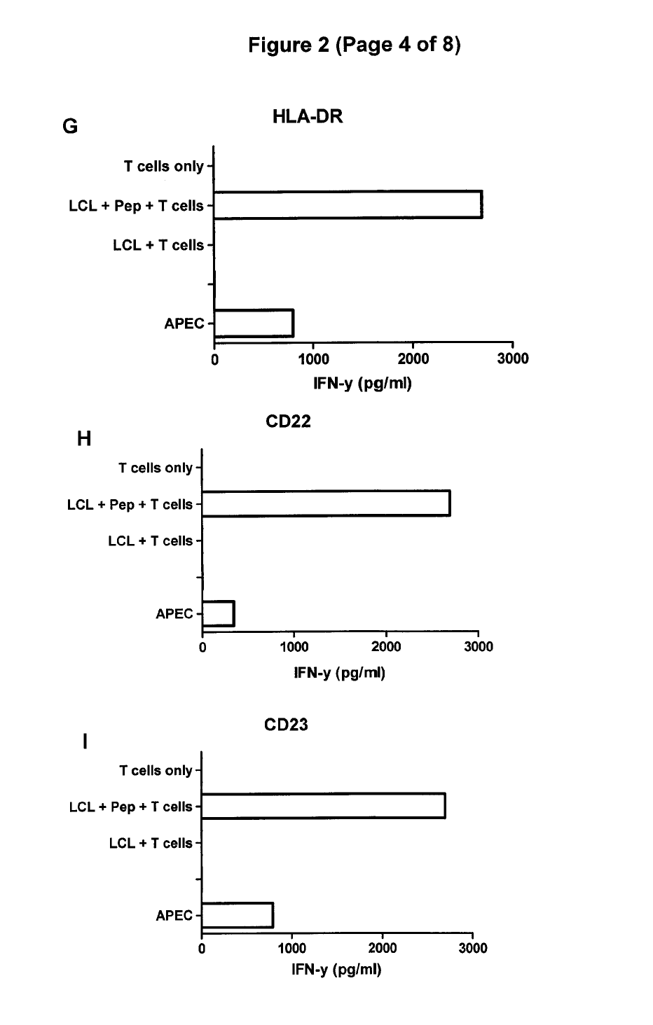

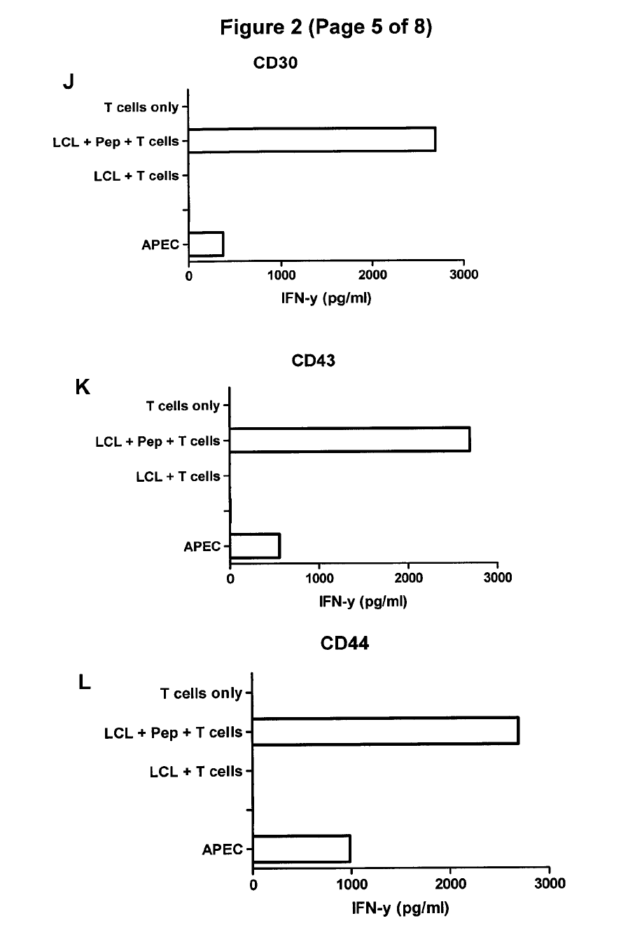

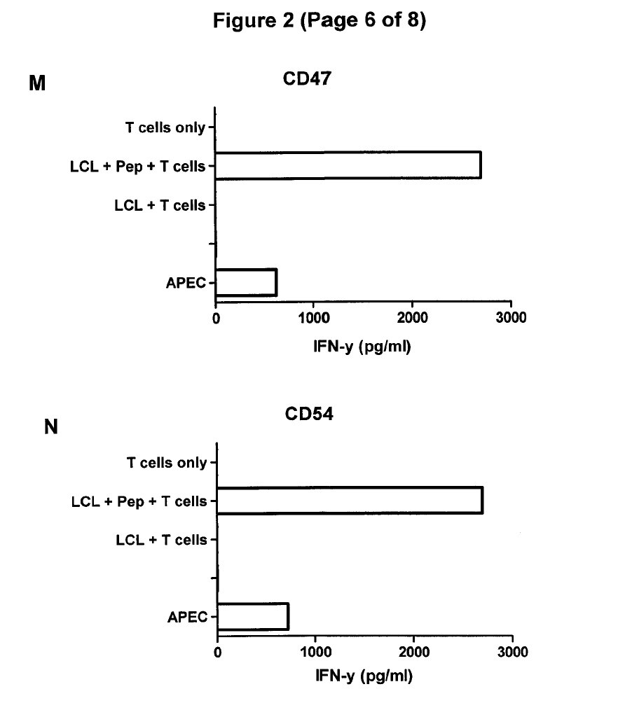

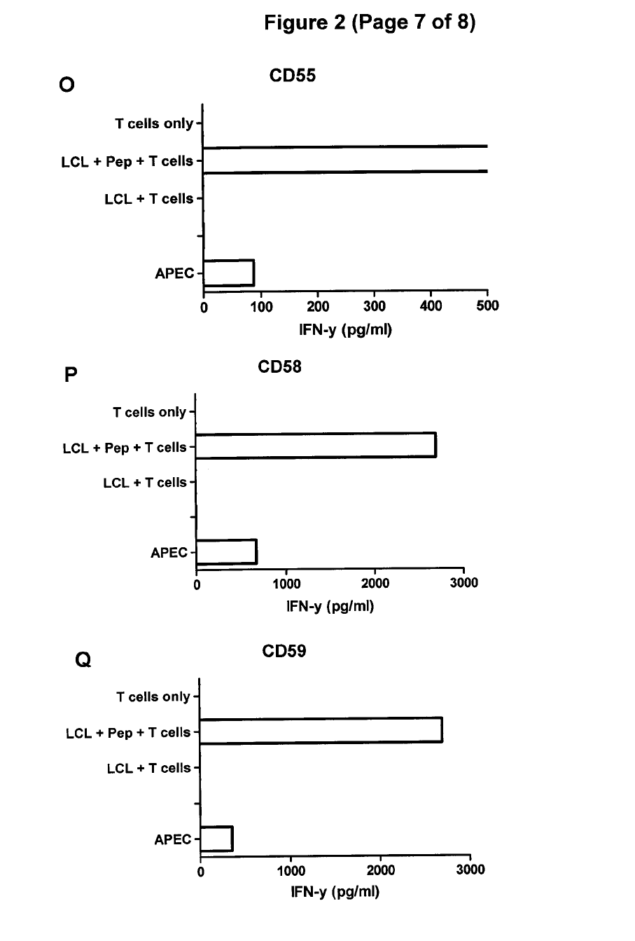

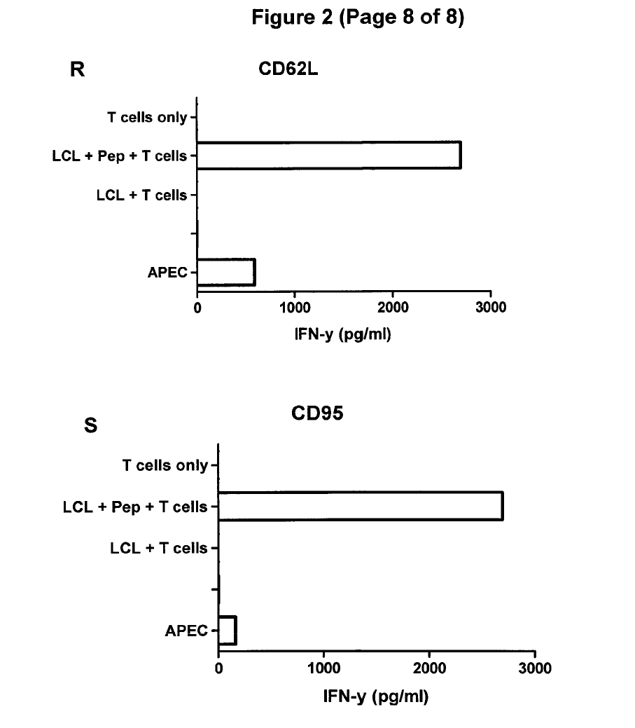

FIG. 2: Data demonstrating in vitro activity of redirected virus-specific T cells after internalisation of antibody peptide-epitope conjugate

(A) Recognition of a lymphoblastoid cell line by CD4+ cytomegalovirus-specific T cells through conjugation of the PDDYSNTHSTRYV (SEQ ID No: 108) peptide to BU69 antibody (anti-CD70, Birmingham University). Recognition of target cells is only present when the antibody is conjugated with the specific peptide. Controls demonstrate specificity of T cells for target cells only in the presence of exogenous peptide.

(B) Cytotoxicity of target cells labelled with BU69 conjugated with the viral peptide DYSNTHSTRYV at a ratio of 5 T cells to 1 target cell. The peptide-conjugated BU69 antibody mediates 40% cytotoxicity compared with 55% cytotoxicity of target cells exogenously pulsed with the viral peptide. There was very little toxicity seen when the BU69 antibody was conjugated with an irrelevant viral peptide.

(C) Recognition of a lymphoblastoid cell line by CD4+ cytomegalovirus-specific T cells through conjugation of the PDDYSNTHSTRYV peptide to anti-CD74 antibody (eBiosciences). Recognition of target cells is only present when the antibody is conjugated with the specific peptide. Controls demonstrate specificity of T cells for target cells only in the presence of exogenous peptide.

(D) Recognition of a lymphoblastoid cell line by CD4+ cytomegalovirus-specific T cells through conjugation of the PDDYSNTHSTRYV peptide to BU69 antibody in presence or absence of the heterobifunctional cross-linker sulfo-SMCC. Recognition of target cells is only present when the antibody is conjugated in the presence of the cross-linker compared with a lack of response when the cross-linker is not present. Controls demonstrate specificity of T cells for target cells only in the presence of exogenous peptide.

(E) Recognition of a lymphoblastoid cell line by CD4+ cytomegalovirus-specific T cells through conjugation of the PDDYSNTHSTRYV peptide to BU69 antibody in presence or absence of inhibitors of the MHC class I or class II antigen processing pathways. Recognition of target cells is reduced only when the cells are cultured in the presence of inhibitors of the MHC class II processing pathway (Chloroquine and Monensin) compared with the untreated control (PDDYSNTHSTRYV). There is no difference in the T cell response towards cells cultured in the presence of inhibitors of the MHC class I processing pathway.

(F) Recognition of a lymphoblastoid cell line by CD4+ Epstein Barr virus-specific T cells through conjugation of the cognate antigen PRSPTVFYNIPPMPLPPSQL peptide to BU69 antibody (anti-CD70, Birmingham University). Recognition of target cells is only present when the antibody is conjugated with the specific peptide. Controls demonstrate specificity of T cells for target cells only in the presence of exogenous peptide.

(G-S) Recognition of a lymphoblastoid cell line by CD4+ cytomegalovirus-specific T cells through recognition of the peptide antigen PDDYSNTHSTRYV conjugated to a secondary antibody. Target cells were first labelled with a primary antibody that could bind to proteins expressed on the surface of the target cells (e.g. CD22, CD23, HLA-DR etc). A secondary antibody (anti-mouse IgG) conjugated with the peptide PDDYSNTHSTRYV is then used to label the antibody bound to the target cells. Target cells labelled with the APEC complex are recognised by peptide-specific T cells as determined by production of IFN-y. Controls demonstrate specificity of T cells for target cells only in the presence of exogenous peptide.

### EXAMPLE 1

### Stimulation of T Cells by Antibody Peptide Epitope Conjugates (APECs)

We have shown that by targeting T cell antigens to particular cell surface targets, the T cell antigen can be internalised and presented on the surface of the cell such that a T cell response is initiated.

FIG. 2A demonstrates T cell recognition of target cells labelled with the agent. The anti-CD70 antibody is conjugated with a peptide (PDDYSNTHSTRYV) or without a peptide (DMSO) and used to label target cells. Cells labelled with the antibody without the peptide are not recognised by the T cells, demonstrated by the absence of IFN-y in the culture supernatant after overnight incubation of the target cells and the T cells. Cells labelled with the antibody conjugated with an immunogenic peptide are strongly recognised by the T cells due to the presence of IFN-y in the culture supernatant. These results suggest that the peptide has been released from the antibody and presented at the cell surface in complex with MHC class II molecules.

The control cells demonstrate that there is no IFN-y release by the target cells alone or in response to incubation with the immunogenic peptide. Also, there is no IFN-y release by T cells in the absence of the immunogenic peptide but once the target cells have been labelled with exogenous immunogenic peptide, there is strong recognition of the T cells demonstrating that the T cells are peptide specific and do not recognise any other peptides naturally expressed by the target cell. Finally, there is no spontaneous release of IFN-y by the T cells throughout the time in culture.

In FIG. 2B, target cells are labelled with an anti-CD70 APEC containing either a control peptide (Biotin-RPHERNFGTVL) or a test peptide (PDDYSNTHSTRYV). The labelled target cells are incubated with peptide-specific T cells for 6 hours and stained with an anti-CD20 antibody for flow cytometric analysis. Target cells labelled with the CD70-PDDYSNTHSTRYV APEC are recognised by the T cells and there is a reduction in the number of target cells left in the well after 6 hours compared with the target cells labelled with the Biotin-RPHERNFGTVL. The result here demonstrates an indirect method of T cell cytotoxicity directed against target cells labelled with APEC.

The control cells were either peptide pulsed or left untreated and cultured with T cells for 6 hours. Analysis on the flow cytometer demonstrated that target cells loaded with exogenous peptide were targeted by the T cells whereas target cells left untreated were ignored by the T cells.

In FIG. 2C target cells are labelled with an anti-CD74 APEC containing either no peptide (DMSO), a control peptide (Biotin-RPHERNFGTVL) or the test peptide (PDDYSNTHSTRYV). After culturing with peptide-specific T cells, target cells labelled with the APEC containing the test peptide were recognised, as demonstrated by the release of IFN-y, whereas the target cells labelled with the APEC without a peptide or with a control peptide were not recognised.

In FIG. 2D anti-CD70 APEC were generated, using DMSO as a no peptide control and the test peptide PDDYSNTHSTRYV, with or without the addition of sulfo-SMCC. This was to demonstrate that the conjugation of the peptide to the antibody is reliant on the hetero-bifunctional cross linker and not via any other chemical interaction. Target cells were labelled with the APEC and cultured with T cells for 16 hours. The supernatant of the cell culture was assayed for the presence of IFN-y. When the conjugation is done in the absence of sulfo-SMCC there is no recognition of the target cells labelled with the control or test APEC suggesting that there is no peptide presented at the surface of the cell. When the conjugation is done in the presence of sulfo-SMCC there is a T cell response to the target cells labelled with the test APEC (PDDYSNTHSTRYV) and no T cell response to the target cells labelled with the control APEC (DMSO). This result demonstrates the requirement for SMCC during conjugation of the peptide to the antibody to generate the APEC.

In FIG. 2E target cells were labelled with an anti-CD70-PDDYSNTHSTRYV APEC in the presence of inhibitors of the HLA class I and class II processing pathways. After addition of T cells to the labelled target cells and subsequent culture for 6 hours, the supernatant was assayed for the presence of IFN-y. Addition of lactacystin, pepstatin or 3-methyladenine (inhibitors of the class I processing pathway) demonstrate a similar level of T cell recognition compared to the cells cultured in the absence of inhibitors (PDDYSNTHSTRYV). Addition of monensin, chloroquine and leupeptin (inhibitors of various aspects of the class II processing pathway) demonstrate a decrease in the amount of IFN-y produced suggesting that the APEC is processed via the HLA class II processing pathway.

In FIG. 2F anti-CD70 APEC were generated using the EBV-derived peptide PRSPTVFYNIPPMPLPPSQL and target cells labelled with the APEC. Peptide-specific T cells were added to the target cells and cultured together for 16 hours and the supernatant assayed for the presence of IFN-y. Target cells labelled with the test APEC (PRSPTVFYNIPPMPLPPSQL) were recognised by T cells whereas target cells labelled with control APEC (DMSO or Biotin-RPHERNFGTVL) were not recognised by the T cells.

Control cells pulsed with exogenous peptide were recognised strongly by the T cells where as untreated target cells were not recognised by the T cells. There was no spontaneous release of IFN-y by T cells when they were cultured alone.

In FIGS. 2G-S, anti-mouse IgG secondary antibody was conjugated with the CMV-derived peptide PDDYSNTHSTRYV. Target cells were first labelled with primary antibodies targeting various cell surface molecules and then labelled a second time using the peptide-conjugated secondary antibody. Peptide-specific T cells were added to the target cells and cultured together for 16 hours and the supernatant assayed for the presence of IFN-y. Target cells labelled with the test APEC after staining with the different primary antibodies were recognised by T cells.

Control cells pulsed with exogenous peptide were recognised strongly by the T cells where as untreated target cells were not recognised by the T cells. There was no spontaneous release of IFN-y by T cells when they were cultured alone.

### EXAMPLE 2

### Standard Operating Procedure for Chemical Conjugation of Cysteinylated Peptide to Antibody

1. Cysteinylated peptides dissolved in DMSO to final concentration of 10 mg/ml.

2. Weigh 1 mg Sulfosuccinimidyl 4-[N-maleimidomethyl]cyclohexane-1-carboxylate (Sulfo-SMCC) and dissolve in 200 μl phosphate buffered saline (PBS).

a. Other heterobifunctional cross-linkers could be used in place of Sulfo-SMCC e.g. Sulfosuccinimidyl 6-(3′-[2-pyridyldithio]-propionamido) hexanoate (Sulfo-LC-SPDP) and N-[β-Maleimidopropionic acid] hydrazide, trifluoroacetic acid salt (BMPH) amongst others.

3. Add 50 μl antibody (10 mg/ml, 500 μg antibody) to dissolved Sulfo-SMCC and incubate at room temperature for 30 minutes.

4. Wash a ZebaSpin Desalting column (7 kDa molecular weight) (Thermo Fisher) by firstly spinning the column at 1,500 g for 1 minute to remove the ethanol (storage buffer).

5. Add 300 μl PBS and spin at 1,500 g for 1 minute. Remove eluate and repeat a further two times.

6. Add 125 μl antibody-SMCC to column, mix well and incubate for 2 minutes.

7. To elute the bound antibody, centrifuge at 1,500 g for 2 minute and collect eluate.

8. Add 5 μl peptide, previously dissolved in DMSO, to the SMCC-activated antibody and incubate at room temperature for 30 minutes.

9. Wash a Protein G column (GE Healthcare) by firstly spinning the column at 13,000 rpm for 30 seconds to remove the ethanol (storage buffer).

10. Add 500 μl PBS and mix protein G beads well before spinning at 13,000 rpm for 30 seconds. Remove eluate and repeat wash a further two times.

11. Add antibody-SMCC to protein G column, mix well and incubate for 5 minutes. Centrifuge at 13,000 rpm for 30 seconds and remove eluate.

12. Wash antibody by adding 500 μl PBS and mixing the beads well before spinning at 13,000 rpm for 30 seconds and removing eluate. Repeat this step a further two times.

13. To elute the bound antibody, add 125 μl 0.1M citric acid to the beads and incubate for 2 minutes at room temperature. Place column in a 1.5 ml eppendorf and spin at 13,000 rpm for 30 seconds and collect eluate.

14. Repeat elution a second time for total elution volume of 250 ul.

15. Add 750 μl 0.2M Na2HCO3 to increase the pH to ˜7. Leave at room temperature for 10 minutes. Antibody-peptide conjugate can now be used to stain cells.

16. Store antibody at 4° C.

```
<160> NUMBER OF SEQ ID NOS: 112
<210> SEQ ID NO 1
<211> LENGTH: 16
<212> TYPE: PRT
<213> ORGANISM: Cytomegalovirus
<400> SEQUENCE: 1
Pro Gln Tyr Ser Glu His Pro Thr Phe Thr Ser Gln Tyr Arg Ile Gln
1 5 10 15
<210> SEQ ID NO 2
<211> LENGTH: 16
<212> TYPE: PRT
<213> ORGANISM: Cytomegalovirus
<400> SEQUENCE: 2
Phe Thr Ser Gln Tyr Arg Ile Gln Gly Lys Leu Glu Tyr Arg His Thr
1 5 10 15
<210> SEQ ID NO 3
<211> LENGTH: 15
<212> TYPE: PRT
<213> ORGANISM: Cytomegalovirus
<400> SEQUENCE: 3
Leu Leu Gln Thr Gly Ile His Val Arg Val Ser Gln Pro Ser Leu
1 5 10 15
<210> SEQ ID NO 4
<211> LENGTH: 15
<212> TYPE: PRT
<213> ORGANISM: Cytomegalovirus
<400> SEQUENCE: 4
Asn Pro Gln Pro Phe Met Arg Pro His Glu Arg Asn Gly Phe Thr
1 5 10 15
<210> SEQ ID NO 5
<211> LENGTH: 15
<212> TYPE: PRT
<213> ORGANISM: Cytomegalovirus
<400> SEQUENCE: 5
Glu Pro Asp Val Tyr Tyr Thr Ser Ala Phe Val Phe Pro Thr Lys
1 5 10 15
<210> SEQ ID NO 6
<211> LENGTH: 15
<212> TYPE: PRT
<213> ORGANISM: Cytomegalovirus
<400> SEQUENCE: 6
Ile Ile Lys Pro Gly Lys Ile Ser His Ile Met Leu Asp Val Ala
1 5 10 15
<210> SEQ ID NO 7
<211> LENGTH: 15
<212> TYPE: PRT
<213> ORGANISM: Cytomegalovirus
<400> SEQUENCE: 7
Ala Gly Ile Leu Ala Arg Asn Leu Val Pro Met Val Ala Thr Val
1 5 10 15
<210> SEQ ID NO 8
<211> LENGTH: 15
<212> TYPE: PRT
<213> ORGANISM: Cytomegalovirus
<400> SEQUENCE: 8
Lys Tyr Gln Glu Phe Phe Trp Asp Ala Asn Asp Ile Tyr Arg Ile
1 5 10 15
<210> SEQ ID NO 9
<211> LENGTH: 11
<212> TYPE: PRT
<213> ORGANISM: Cytomegalovirus
<400> SEQUENCE: 9
Asp Tyr Ser Asn Thr His Ser Thr Arg Tyr Val
1 5 10
<210> SEQ ID NO 10
<211> LENGTH: 15
<212> TYPE: PRT
<213> ORGANISM: Cytomegalovirus
<400> SEQUENCE: 10
Cys Met Leu Thr Ile Thr Thr Ala Arg Ser Lys Tyr Pro Tyr His
1 5 10 15
<210> SEQ ID NO 11
<211> LENGTH: 15
<212> TYPE: PRT
<213> ORGANISM: Cytomegalovirus
<400> SEQUENCE: 11
Val Phe Glu Thr Ser Gly Gly Leu Val Val Phe Trp Gln Gly Ile
1 5 10 15
<210> SEQ ID NO 12
<211> LENGTH: 20
<212> TYPE: PRT
<213> ORGANISM: Cytomegalovirus
<400> SEQUENCE: 12
Val Arg Val Asp Met Val Arg His Arg Ile Lys Glu His Met Leu Lys
1 5 10 15
Lys Tyr Thr Gln
                        20
<210> SEQ ID NO 13
<211> LENGTH: 20
<212> TYPE: PRT
<213> ORGANISM: Cytomegalovirus
<400> SEQUENCE: 13
Asn Tyr Ile Val Pro Glu Asp Lys Arg Glu Met Trp Met Ala Cys Ile
1 5 10 15
Lys Glu Leu His
                        20
<210> SEQ ID NO 14
<211> LENGTH: 12
<212> TYPE: PRT
<213> ORGANISM: Cytomegalovirus
<400> SEQUENCE: 14
His Glu Leu Leu Val Leu Val Lys Lys Ala Gln Leu
1 5 10
<210> SEQ ID NO 15
<211> LENGTH: 15
<212> TYPE: PRT
<213> ORGANISM: Epstein Barr virus
<400> SEQUENCE: 15
Arg Arg Pro Gln Lys Arg Pro Ser Cys Ile Gly Cys Lys Gly Thr
1 5 10 15
<210> SEQ ID NO 16
<211> LENGTH: 15
<212> TYPE: PRT
<213> ORGANISM: Epstein Barr virus
<400> SEQUENCE: 16
Arg Pro Phe Phe His Pro Val Gly Glu Ala Asp Tyr Phe Glu Tyr
1 5 10 15
<210> SEQ ID NO 17
<211> LENGTH: 20
<212> TYPE: PRT
<213> ORGANISM: Epstein Barr virus
<400> SEQUENCE: 17
Val Pro Pro Gly Ala Ile Glu Gln Gly Pro Ala Asp Asp Pro Gly Glu
1 5 10 15
Gly Pro Ser Thr
                        20
<210> SEQ ID NO 18
<211> LENGTH: 25
<212> TYPE: PRT
<213> ORGANISM: Epstein Barr virus
<400> SEQUENCE: 18
Ile Glu Gln Gly Pro Thr Asp Asp Pro Gly Glu Gly Pro Ser Thr Gly
1 5 10 15
Pro Arg Gly Gln Gly Asp Gly Gly Arg
                        20 25
<210> SEQ ID NO 19
<211> LENGTH: 15
<212> TYPE: PRT
<213> ORGANISM: Epstein Barr virus
<400> SEQUENCE: 19
Asp Gly Gly Arg Arg Lys Lys Gly Gly Trp Phe Gly Arg His Arg
1 5 10 15
<210> SEQ ID NO 20
<211> LENGTH: 20
<212> TYPE: PRT
<213> ORGANISM: Epstein Barr virus
<400> SEQUENCE: 20
Ser Asn Pro Lys Phe Glu Asn Ile Ala Glu Gly Leu Arg Val Leu Leu
1 5 10 15
Ala Arg Ser His
                        20
<210> SEQ ID NO 21
<211> LENGTH: 15
<212> TYPE: PRT
<213> ORGANISM: Epstein Barr virus
<400> SEQUENCE: 21
Asn Pro Lys Phe Glu Asn Ile Ala Glu Gly Leu Arg Ala Leu Leu
1 5 10 15
<210> SEQ ID NO 22
<211> LENGTH: 20
<212> TYPE: PRT
<213> ORGANISM: Epstein Barr virus
<400> SEQUENCE: 22
Glu Asn Ile Ala Glu Gly Leu Arg Val Leu Leu Ala Arg Ser His Val
1 5 10 15
Glu Arg Thr Thr
                        20
<210> SEQ ID NO 23
<211> LENGTH: 20
<212> TYPE: PRT
<213> ORGANISM: Epstein Barr virus
<400> SEQUENCE: 23
Ile Ala Glu Gly Leu Arg Ala Leu Leu Ala Arg Ser His Val Glu Arg
1 5 10 15
Thr Thr Asp Glu
                        20
<210> SEQ ID NO 24
<211> LENGTH: 15
<212> TYPE: PRT
<213> ORGANISM: Epstein Barr virus
<400> SEQUENCE: 24
Leu Arg Ala Leu Leu Ala Arg Ser His Val Glu Arg Thr Thr Asp
1 5 10 15
<210> SEQ ID NO 25
<211> LENGTH: 25
<212> TYPE: PRT
<213> ORGANISM: Epstein Barr virus
<400> SEQUENCE: 25
Glu Glu Gly Asn Trp Val Ala Gly Val Phe Val Tyr Gly Gly Ser Lys
1 5 10 15
Thr Ser Leu Tyr Asn Leu Arg Arg Gly
                        20 25
<210> SEQ ID NO 26
<211> LENGTH: 20
<212> TYPE: PRT
<213> ORGANISM: Epstein Barr virus
<400> SEQUENCE: 26
Val Tyr Gly Gly Ser Lys Thr Ser Leu Tyr Asn Leu Arg Arg Gly Thr
1 5 10 15
Ala Leu Ala Ile
                        20
<210> SEQ ID NO 27
<211> LENGTH: 14
<212> TYPE: PRT
<213> ORGANISM: Epstein Barr virus
<400> SEQUENCE: 27
Thr Ser Leu Tyr Asn Leu Arg Arg Gly Thr Ala Leu Ala Ile
1 5 10
<210> SEQ ID NO 28
<211> LENGTH: 13
<212> TYPE: PRT
<213> ORGANISM: Epstein Barr virus
<400> SEQUENCE: 28
Tyr Asn Leu Arg Arg Gly Thr Ala Leu Ala Ile Pro Gln
1 5 10
<210> SEQ ID NO 29
<211> LENGTH: 16
<212> TYPE: PRT
<213> ORGANISM: Epstein Barr virus
<400> SEQUENCE: 29
Asn Leu Arg Arg Gly Arg Thr Ala Leu Ala Ile Pro Gln Cys Arg Leu
1 5 10 15
<210> SEQ ID NO 30
<211> LENGTH: 25
<212> TYPE: PRT
<213> ORGANISM: Epstein Barr virus
<400> SEQUENCE: 30
Glu Glu Gly Asn Trp Val Ala Gly Val Phe Val Tyr Gly Gly Ser Lys
1 5 10 15
Thr Ser Leu Tyr Asn Leu Arg Arg Gly
                        20 25
<210> SEQ ID NO 31
<211> LENGTH: 15
<212> TYPE: PRT
<213> ORGANISM: Epstein Barr virus
<400> SEQUENCE: 31
Ala Ile Pro Gln Cys Arg Leu Thr Pro Leu Ser Arg Leu Pro Phe
1 5 10 15
<210> SEQ ID NO 32
<211> LENGTH: 15
<212> TYPE: PRT
<213> ORGANISM: Epstein Barr virus
<400> SEQUENCE: 32
Pro Gln Cys Arg Leu Thr Pro Leu Ser Arg Leu Pro Phe Gly Met
1 5 10 15
<210> SEQ ID NO 33
<211> LENGTH: 18
<212> TYPE: PRT
<213> ORGANISM: Epstein Barr virus
<400> SEQUENCE: 33
Ala Pro Gly Pro Gly Pro Gln Pro Leu Arg Glu Ser Ile Val Cys Tyr
1 5 10 15
Phe Met
<210> SEQ ID NO 34
<211> LENGTH: 20
<212> TYPE: PRT
<213> ORGANISM: Epstein Barr virus
<400> SEQUENCE: 34
Pro Gln Pro Gly Pro Leu Arg Glu Ser Ile Val Cys Tyr Phe Met Val
1 5 10 15
Phe Leu Gln Thr
                        20
<210> SEQ ID NO 35
<211> LENGTH: 20
<212> TYPE: PRT
<213> ORGANISM: Epstein Barr virus
<400> SEQUENCE: 35
Pro Gly Pro Leu Arg Glu Ser Ile Val Cys Tyr Phe Met Val Phe Leu
1 5 10 15
Gln Thr His Ile
                        20
<210> SEQ ID NO 36
<211> LENGTH: 20
<212> TYPE: PRT
<213> ORGANISM: Epstein Barr virus
<400> SEQUENCE: 36
Leu Arg Glu Ser Ile Val Cys Tyr Phe Met Val Phe Leu Gln Thr His
1 5 10 15
Ile Phe Ala Glu
                        20
<210> SEQ ID NO 37
<211> LENGTH: 25
<212> TYPE: PRT
<213> ORGANISM: Epstein Barr virus
<400> SEQUENCE: 37
Leu Arg Glu Ser Ile Val Cys Tyr Phe Met Val Phe Leu Gln Thr His
1 5 10 15
Ile Phe Ala Glu Val Leu Lys Asp Ala
                        20 25
<210> SEQ ID NO 38
<211> LENGTH: 13
<212> TYPE: PRT
<213> ORGANISM: Epstein Barr virus
<400> SEQUENCE: 38
Tyr Phe Met Val Phe Leu Gln Thr His Ile Phe Ala Glu
1 5 10
<210> SEQ ID NO 39
<211> LENGTH: 15
<212> TYPE: PRT
<213> ORGANISM: Epstein Barr virus
<400> SEQUENCE: 39
Met Val Phe Leu Gln Thr His Ile Phe Ala Glu Val Leu Lys Asp
1 5 10 15
<210> SEQ ID NO 40
<211> LENGTH: 19
<212> TYPE: PRT
<213> ORGANISM: Epstein Barr virus
<400> SEQUENCE: 40
Val Phe Leu Gln Thr His Ile Phe Ala Glu Val Leu Lys Asp Ala Ile
1 5 10 15
Lys Asp Leu
<210> SEQ ID NO 41
<211> LENGTH: 20
<212> TYPE: PRT
<213> ORGANISM: Epstein Barr virus
<400> SEQUENCE: 41
Val Leu Lys Asp Ala Ile Lys Asp Leu Val Met Thr Lys Pro Ala Pro
1 5 10 15
Thr Cys Asn Ile
                        20
<210> SEQ ID NO 42
<211> LENGTH: 25
<212> TYPE: PRT
<213> ORGANISM: Epstein Barr virus
<400> SEQUENCE: 42
Pro Thr Cys Asn Ile Lys Val Thr Val Cys Ser Phe Asp Asp Gly Val
1 5 10 15
Asp Leu Pro Pro Trp Phe Pro Pro Met
                        20 25
<210> SEQ ID NO 43
<211> LENGTH: 20
<212> TYPE: PRT
<213> ORGANISM: Epstein Barr virus
<400> SEQUENCE: 43
Arg Val Thr Val Cys Ser Phe Asp Asp Gly Val Asp Leu Pro Pro Trp
1 5 10 15
Phe Pro Pro Met
                        20
<210> SEQ ID NO 44
<211> LENGTH: 13
<212> TYPE: PRT
<213> ORGANISM: Epstein Barr virus
<400> SEQUENCE: 44
Pro Pro Trp Phe Pro Pro Met Val Glu Gly Ala Ala Ala
1 5 10
<210> SEQ ID NO 45
<211> LENGTH: 20
<212> TYPE: PRT
<213> ORGANISM: Epstein Barr virus
<400> SEQUENCE: 45
Gly Gln Thr Tyr His Leu Ile Val Asp Thr Leu Ala Leu His Gly Gly
1 5 10 15
Gln Thr Tyr His
                        20
<210> SEQ ID NO 46
<211> LENGTH: 20
<212> TYPE: PRT
<213> ORGANISM: Epstein Barr virus
<400> SEQUENCE: 46
Ile Pro Leu Thr Ile Phe Val Gly Glu Asn Thr Gly Val Pro Pro Pro
1 5 10 15
Leu Pro Pro Pro
                        20
<210> SEQ ID NO 47
<211> LENGTH: 20
<212> TYPE: PRT
<213> ORGANISM: Epstein Barr virus
<400> SEQUENCE: 47
Met Arg Met Leu Trp Met Ala Asn Tyr Ile Val Arg Gln Ser Arg Gly
1 5 10 15
Asp Arg Gly Leu
                        20
<210> SEQ ID NO 48
<211> LENGTH: 19
<212> TYPE: PRT
<213> ORGANISM: Epstein Barr virus
<400> SEQUENCE: 48
Leu Pro Pro Ala Thr Leu Val Pro Pro Arg Pro Thr Arg Pro Thr Thr
1 5 10 15
Leu Pro Pro
<210> SEQ ID NO 49
<211> LENGTH: 20
<212> TYPE: PRT
<213> ORGANISM: Epstein Barr virus
<400> SEQUENCE: 49
Pro Arg Ser Pro Thr Val Phe Tyr Asn Ile Pro Pro Met Pro Leu Pro
1 5 10 15
Pro Ser Gln Leu
                        20
<210> SEQ ID NO 50
<211> LENGTH: 11
<212> TYPE: PRT
<213> ORGANISM: Epstein Barr virus
<400> SEQUENCE: 50
Thr Val Phe Tyr Asn Ile Pro Pro Met Pro Leu
1 5 10
<210> SEQ ID NO 51
<211> LENGTH: 20
<212> TYPE: PRT
<213> ORGANISM: Epstein Barr virus
<400> SEQUENCE: 51
Pro Ala Gln Pro Pro Pro Gly Val Ile Asn Asp Gln Gln Leu His His
1 5 10 15
Leu Pro Ser Gly
                        20
<210> SEQ ID NO 52
<211> LENGTH: 20
<212> TYPE: PRT
<213> ORGANISM: Epstein Barr virus
<400> SEQUENCE: 52
Glu Asp Leu Pro Cys Ile Val Ser Arg Gly Gly Pro Lys Val Lys Arg
1 5 10 15
Pro Pro Ile Phe
                        20
<210> SEQ ID NO 53
<211> LENGTH: 20
<212> TYPE: PRT
<213> ORGANISM: Epstein Barr virus
<400> SEQUENCE: 53
Gly Pro Trp Val Pro Glu Gln Trp Met Phe Gln Gly Ala Pro Pro Ser
1 5 10 15
Gln Gly Thr Pro
                        20
<210> SEQ ID NO 54
<211> LENGTH: 19
<212> TYPE: PRT
<213> ORGANISM: Epstein Barr virus
<400> SEQUENCE: 54
Gln Val Ala Asp Val Val Arg Ala Pro Gly Val Pro Ala Met Gln Pro
1 5 10 15
Gln Tyr Phe
<210> SEQ ID NO 55
<211> LENGTH: 14
<212> TYPE: PRT
<213> ORGANISM: Epstein Barr virus
<400> SEQUENCE: 55
Asn Arg Gly Trp Met Gln Arg Ile Arg Arg Arg Arg Arg Arg
1 5 10
<210> SEQ ID NO 56
<211> LENGTH: 21
<212> TYPE: PRT
<213> ORGANISM: Epstein Barr virus
<400> SEQUENCE: 56
Pro His Asp Ile Thr Tyr Pro Tyr Thr Ala Arg Asn Ile Arg Asp Ala
1 5 10 15
Ala Cys Arg Ala Val
                        20
<210> SEQ ID NO 57
<211> LENGTH: 15
<212> TYPE: PRT
<213> ORGANISM: Epstein Barr virus
<400> SEQUENCE: 57
Ile Leu Cys Phe Val Met Ala Ala Arg Gln Arg Leu Gln Asp Ile
1 5 10 15
<210> SEQ ID NO 58
<211> LENGTH: 15
<212> TYPE: PRT
<213> ORGANISM: Epstein Barr virus
<400> SEQUENCE: 58
Ser Asp Asp Glu Leu Pro Tyr Ile Asp Pro Asn Met Glu Pro Val
1 5 10 15
<210> SEQ ID NO 59
<211> LENGTH: 15
<212> TYPE: PRT
<213> ORGANISM: Epstein Barr virus
<400> SEQUENCE: 59
Gln Gln Arg Pro Val Met Phe Val Ser Arg Val Pro Ala Lys Lys
1 5 10 15
<210> SEQ ID NO 60
<211> LENGTH: 15
<212> TYPE: PRT
<213> ORGANISM: Epstein Barr virus
<400> SEQUENCE: 60
Gln Lys Arg Ala Ala Pro Pro Thr Val Ser Pro Ser Asp Thr Gly
1 5 10 15
<210> SEQ ID NO 61
<211> LENGTH: 15
<212> TYPE: PRT
<213> ORGANISM: Epstein Barr virus
<400> SEQUENCE: 61
Pro Pro Ala Ala Gly Pro Pro Ala Ala Gly Pro Arg Ile Leu Ala
1 5 10 15
<210> SEQ ID NO 62
<211> LENGTH: 15
<212> TYPE: PRT
<213> ORGANISM: Epstein Barr virus
<400> SEQUENCE: 62
Pro Pro Val Val Arg Met Phe Met Arg Glu Arg Gln Leu Pro Gln
1 5 10 15
<210> SEQ ID NO 63
<211> LENGTH: 15
<212> TYPE: PRT
<213> ORGANISM: Epstein Barr virus
<400> SEQUENCE: 63
Pro Gln Cys Phe Trp Glu Met Arg Ala Gly Arg Glu Ile Thr Gln
1 5 10 15
<210> SEQ ID NO 64
<211> LENGTH: 20
<212> TYPE: PRT
<213> ORGANISM: Epstein Barr virus
<400> SEQUENCE: 64
Pro Ala Pro Gln Ala Pro Tyr Gln Gly Tyr Gln Glu Pro Pro Ala Pro
1 5 10 15
Gln Ala Pro Tyr
                        20
<210> SEQ ID NO 65
<211> LENGTH: 15
<212> TYPE: PRT
<213> ORGANISM: Epstein Barr virus
<400> SEQUENCE: 65
Pro Ser Met Pro Phe Ala Ser Asp Tyr Ser Gln Gly Ala Phe Thr
1 5 10 15
<210> SEQ ID NO 66
<211> LENGTH: 16
<212> TYPE: PRT
<213> ORGANISM: Epstein Barr virus
<400> SEQUENCE: 66
Ala Gln Glu Ile Leu Ser Asp Asn Ser Glu Ile Ser Val Phe Pro Lys
1 5 10 15
<210> SEQ ID NO 67
<211> LENGTH: 20
<212> TYPE: PRT
<213> ORGANISM: Epstein Barr virus
<400> SEQUENCE: 67
Gly Pro Pro Arg Pro Pro Leu Gly Pro Pro Leu Ser Ser Ser Ile Gly
1 5 10 15
Leu Ala Leu Leu
                        20
<210> SEQ ID NO 68
<211> LENGTH: 15
<212> TYPE: PRT
<213> ORGANISM: Epstein Barr virus
<400> SEQUENCE: 68
Leu Trp Arg Leu Gly Ala Thr Ile Trp Gln Leu Leu Ala Phe Phe
1 5 10 15
<210> SEQ ID NO 69
<211> LENGTH: 20
<212> TYPE: PRT
<213> ORGANISM: Epstein Barr virus
<400> SEQUENCE: 69
Leu Ile Trp Met Tyr Tyr His Gly Pro Arg His Thr Asp Glu His His
1 5 10 15
His Asp Asp Ser
                        20
<210> SEQ ID NO 70
<211> LENGTH: 20
<212> TYPE: PRT
<213> ORGANISM: Epstein Barr virus
<400> SEQUENCE: 70
Gln Ala Thr Asp Asp Ser Ser His Glu Ser Asp Ser Asn Ser Asn Glu
1 5 10 15
Gly Arg His His
                        20
<210> SEQ ID NO 71
<211> LENGTH: 20
<212> TYPE: PRT
<213> ORGANISM: Epstein Barr virus
<400> SEQUENCE: 71
Ser Ser His Glu Ser Asp Ser Asn Ser Asn Glu Gly Arg His His Leu
1 5 10 15
Leu Val Ser Gly
                        20
<210> SEQ ID NO 72
<211> LENGTH: 16
<212> TYPE: PRT
<213> ORGANISM: Epstein Barr virus
<400> SEQUENCE: 72
Ser Gly His Glu Ser Asp Ser Asn Ser Asn Glu Gly Arg His His His
1 5 10 15
<210> SEQ ID NO 73
<211> LENGTH: 15
<212> TYPE: PRT
<213> ORGANISM: Epstein Barr virus
<400> SEQUENCE: 73
Thr Asp Gly Gly Gly Gly His Ser His Asp Ser Gly His Gly Gly
1 5 10 15
<210> SEQ ID NO 74
<211> LENGTH: 15
<212> TYPE: PRT
<213> ORGANISM: Epstein Barr virus
<400> SEQUENCE: 74
Asp Tyr Gln Pro Leu Gly Thr Gln Asp Gln Ser Leu Tyr Leu Gly
1 5 10 15
<210> SEQ ID NO 75
<211> LENGTH: 15
<212> TYPE: PRT
<213> ORGANISM: Epstein Barr virus
<400> SEQUENCE: 75
Ser Thr Val Val Thr Ala Thr Gly Leu Ala Leu Ser Leu Leu Leu
1 5 10 15
<210> SEQ ID NO 76
<211> LENGTH: 14
<212> TYPE: PRT
<213> ORGANISM: Epstein Barr virus
<400> SEQUENCE: 76
Ser Ser Tyr Ala Ala Ala Gln Arg Lys Leu Leu Thr Pro Val
1 5 10
<210> SEQ ID NO 77
<211> LENGTH: 20
<212> TYPE: PRT
<213> ORGANISM: Epstein Barr virus
<400> SEQUENCE: 77
Val Thr Phe Phe Ala Ile Cys Leu Thr Trp Arg Ile Glu Asp Pro Pro
1 5 10 15
Phe Asn Ser Ile
                        20
<210> SEQ ID NO 78
<211> LENGTH: 20
<212> TYPE: PRT
<213> ORGANISM: Epstein Barr virus
<400> SEQUENCE: 78
Ile Cys Leu Thr Trp Arg Ile Glu Asp Pro Pro Phe Asn Ser Ile Leu
1 5 10 15
Phe Ala Leu Leu
                        20
<210> SEQ ID NO 79
<211> LENGTH: 20
<212> TYPE: PRT
<213> ORGANISM: Epstein Barr virus
<400> SEQUENCE: 79
Val Leu Val Met Leu Val Leu Leu Ile Leu Ala Tyr Arg Arg Arg Trp
1 5 10 15
Arg Arg Leu Thr
                        20
<210> SEQ ID NO 80
<211> LENGTH: 14
<212> TYPE: PRT
<213> ORGANISM: Epstein Barr virus
<400> SEQUENCE: 80
Ser Thr Glu Phe Ile Pro Asn Leu Phe Cys Met Leu Leu Leu
1 5 10
<210> SEQ ID NO 81
<211> LENGTH: 20
<212> TYPE: PRT
<213> ORGANISM: Epstein Barr virus
<400> SEQUENCE: 81
Thr Tyr Gly Pro Val Phe Met Ser Leu Gly Gly Leu Leu Thr Met Val
1 5 10 15
Ala Gly Ala Val
                        20
<210> SEQ ID NO 82
<211> LENGTH: 19
<212> TYPE: PRT
<213> ORGANISM: Epstein Barr virus
<400> SEQUENCE: 82
Ala Gly Leu Thr Leu Ser Leu Leu Val Ile Cys Ser Tyr Leu Phe Ile
1 5 10 15
Ser Arg Gly
<210> SEQ ID NO 83
<211> LENGTH: 12
<212> TYPE: PRT
<213> ORGANISM: Epstein Barr virus
<400> SEQUENCE: 83
Pro Tyr Tyr Val Val Asp Leu Ser Val Arg Gly Met
1 5 10
<210> SEQ ID NO 84
<211> LENGTH: 13
<212> TYPE: PRT
<213> ORGANISM: Epstein Barr virus
<400> SEQUENCE: 84
Thr Val Val Leu Arg Tyr His Val Leu Leu Glu Glu Ile
1 5 10
<210> SEQ ID NO 85
<211> LENGTH: 15
<212> TYPE: PRT
<213> ORGANISM: Epstein Barr virus
<400> SEQUENCE: 85
Glu Leu Glu Ile Lys Arg Tyr Lys Asn Arg Val Ala Ser Arg Lys
1 5 10 15
<210> SEQ ID NO 86
<211> LENGTH: 15
<212> TYPE: PRT
<213> ORGANISM: Epstein Barr virus
<400> SEQUENCE: 86
Lys Ser Ser Glu Asn Asp Arg Leu Arg Leu Leu Leu Lys Gln Met
1 5 10 15
<210> SEQ ID NO 87
<211> LENGTH: 20
<212> TYPE: PRT
<213> ORGANISM: Epstein Barr virus
<400> SEQUENCE: 87
Leu Asp Leu Phe Gly Gln Leu Thr Pro His Thr Lys Ala Val Tyr Gln
1 5 10 15
Pro Arg Gly Ala
                        20
<210> SEQ ID NO 88
<211> LENGTH: 15
<212> TYPE: PRT
<213> ORGANISM: Epstein Barr virus
<400> SEQUENCE: 88
Phe Gly Gln Leu Thr Pro His Thr Lys Ala Val Tyr Gln Pro Arg
1 5 10 15
<210> SEQ ID NO 89
<211> LENGTH: 15
<212> TYPE: PRT
<213> ORGANISM: Epstein Barr virus
<400> SEQUENCE: 89
Val Tyr Phe Gln Asp Val Phe Gly Thr Met Trp Cys His His Ala
1 5 10 15
<210> SEQ ID NO 90
<211> LENGTH: 21
<212> TYPE: PRT
<213> ORGANISM: Epstein Barr virus
<400> SEQUENCE: 90
Asp Asn Cys Asn Ser Thr Asn Ile Thr Ala Val Val Arg Ala Gln Gly
1 5 10 15
Leu Asp Val Thr Leu
                        20
<210> SEQ ID NO 91
<211> LENGTH: 15
<212> TYPE: PRT
<213> ORGANISM: Epstein Barr virus
<400> SEQUENCE: 91
Ala Trp Cys Leu Glu Gln Lys Arg Gln Asn Met Val Leu Arg Glu
1 5 10 15
<210> SEQ ID NO 92
<211> LENGTH: 15
<212> TYPE: PRT
<213> ORGANISM: Epstein Barr virus
<400> SEQUENCE: 92
Asp Asn Glu Ile Phe Leu Thr Lys Lys Met Thr Glu Val Cys Gln
1 5 10 15
<210> SEQ ID NO 93
<211> LENGTH: 193
<212> TYPE: PRT
<213> ORGANISM: Homo sapiens
<220> FEATURE:
<223> OTHER INFORMATION: CD70
<400> SEQUENCE: 93
Met Pro Glu Glu Gly Ser Gly Cys Ser Val Arg Arg Arg Pro Tyr Gly
1 5 10 15
Cys Val Leu Arg Ala Ala Leu Val Pro Leu Val Ala Gly Leu Val Ile
                        20 25 30
Cys Leu Val Val Cys Ile Gln Arg Phe Ala Gln Ala Gln Gln Gln Leu
                35 40 45
Pro Leu Glu Ser Leu Gly Trp Asp Val Ala Glu Leu Gln Leu Asn His
        50 55 60
Thr Gly Pro Gln Gln Asp Pro Arg Leu Tyr Trp Gln Gly Gly Pro Ala
65 70 75 80
Leu Gly Arg Ser Phe Leu His Gly Pro Glu Leu Asp Lys Gly Gln Leu
                                85 90 95
Arg Ile His Arg Asp Gly Ile Tyr Met Val His Ile Gln Val Thr Leu
                        100 105 110
Ala Ile Cys Ser Ser Thr Thr Ala Ser Arg His His Pro Thr Thr Leu
                115 120 125
Ala Val Gly Ile Cys Ser Pro Ala Ser Arg Ser Ile Ser Leu Leu Arg
        130 135 140
Leu Ser Phe His Gln Gly Cys Thr Ile Ala Ser Gln Arg Leu Thr Pro
145 150 155 160
Leu Ala Arg Gly Asp Thr Leu Cys Thr Asn Leu Thr Gly Thr Leu Leu
                                165 170 175
Pro Ser Arg Asn Thr Asp Glu Thr Phe Phe Gly Val Gln Trp Val Arg
                        180 185 190
Pro
<210> SEQ ID NO 94
<211> LENGTH: 296
<212> TYPE: PRT
<213> ORGANISM: Homo sapiens
<220> FEATURE:
<223> OTHER INFORMATION: CD74
<400> SEQUENCE: 94
Met His Arg Arg Arg Ser Arg Ser Cys Arg Glu Asp Gln Lys Pro Val
1 5 10 15
Met Asp Asp Gln Arg Asp Leu Ile Ser Asn Asn Glu Gln Leu Pro Met
                        20 25 30
Leu Gly Arg Arg Pro Gly Ala Pro Glu Ser Lys Cys Ser Arg Gly Ala
                35 40 45
Leu Tyr Thr Gly Phe Ser Ile Leu Val Thr Leu Leu Leu Ala Gly Gln
        50 55 60
Ala Thr Thr Ala Tyr Phe Leu Tyr Gln Gln Gln Gly Arg Leu Asp Lys
65 70 75 80
Leu Thr Val Thr Ser Gln Asn Leu Gln Leu Glu Asn Leu Arg Met Lys
                                85 90 95
Leu Pro Lys Pro Pro Lys Pro Val Ser Lys Met Arg Met Ala Thr Pro
                        100 105 110
Leu Leu Met Gln Ala Leu Pro Met Gly Ala Leu Pro Gln Gly Pro Met
                115 120 125
Gln Asn Ala Thr Lys Tyr Gly Asn Met Thr Glu Asp His Val Met His
        130 135 140
Leu Leu Gln Asn Ala Asp Pro Leu Lys Val Tyr Pro Pro Leu Lys Gly
145 150 155 160
Ser Phe Pro Glu Asn Leu Arg His Leu Lys Asn Thr Met Glu Thr Ile
                                165 170 175
Asp Trp Lys Val Phe Glu Ser Trp Met His His Trp Leu Leu Phe Glu
                        180 185 190
Met Ser Arg His Ser Leu Glu Gln Lys Pro Thr Asp Ala Pro Pro Lys
                195 200 205
Val Leu Thr Lys Cys Gln Glu Glu Val Ser His Ile Pro Ala Val His
        210 215 220
Pro Gly Ser Phe Arg Pro Lys Cys Asp Glu Asn Gly Asn Tyr Leu Pro
225 230 235 240
Leu Gln Cys Tyr Gly Ser Ile Gly Tyr Cys Trp Cys Val Phe Pro Asn
                                245 250 255
Gly Thr Glu Val Pro Asn Thr Arg Ser Arg Gly His His Asn Cys Ser
                        260 265 270
Glu Ser Leu Glu Leu Glu Asp Pro Ser Ser Gly Leu Gly Val Thr Lys
                275 280 285
Gln Asp Leu Gly Pro Val Pro Met
        290 295
<210> SEQ ID NO 95
<211> LENGTH: 254
<212> TYPE: PRT
<213> ORGANISM: Homo sapiens
<220> FEATURE:
<223> OTHER INFORMATION: HLA-DR alpha
<400> SEQUENCE: 95
Met Ala Ile Ser Gly Val Pro Val Leu Gly Phe Phe Ile Ile Ala Val
1 5 10 15
Leu Met Ser Ala Gln Glu Ser Trp Ala Ile Lys Glu Glu His Val Ile
                        20 25 30
Ile Gln Ala Glu Phe Tyr Leu Asn Pro Asp Gln Ser Gly Glu Phe Met
                35 40 45
Phe Asp Phe Asp Gly Asp Glu Ile Phe His Val Asp Met Ala Lys Lys
        50 55 60
Glu Thr Val Trp Arg Leu Glu Glu Phe Gly Arg Phe Ala Ser Phe Glu
65 70 75 80
Ala Gln Gly Ala Leu Ala Asn Ile Ala Val Asp Lys Ala Asn Leu Glu
                                85 90 95
Ile Met Thr Lys Arg Ser Asn Tyr Thr Pro Ile Thr Asn Val Pro Pro
                        100 105 110
Glu Val Thr Val Leu Thr Asn Ser Pro Val Glu Leu Arg Glu Pro Asn
                115 120 125
Val Leu Ile Cys Phe Ile Asp Lys Phe Thr Pro Pro Val Val Asn Val
        130 135 140
Thr Trp Leu Arg Asn Gly Lys Pro Val Thr Thr Gly Val Ser Glu Thr
145 150 155 160
Val Phe Leu Pro Arg Glu Asp His Leu Phe Arg Lys Phe His Tyr Leu
                                165 170 175
Pro Phe Leu Pro Ser Thr Glu Asp Val Tyr Asp Cys Arg Val Glu His
                        180 185 190
Trp Gly Leu Asp Glu Pro Leu Leu Lys His Trp Glu Phe Asp Ala Pro
                195 200 205
Ser Pro Leu Pro Glu Thr Thr Glu Asn Val Val Cys Ala Leu Gly Leu
        210 215 220
Thr Val Gly Leu Val Gly Ile Ile Ile Gly Thr Ile Phe Ile Ile Lys
225 230 235 240
Gly Val Arg Lys Ser Asn Ala Ala Glu Arg Arg Gly Pro Leu
                                245 250
<210> SEQ ID NO 96
<211> LENGTH: 480
<212> TYPE: PRT
<213> ORGANISM: Homo sapiens
<220> FEATURE:
<223> OTHER INFORMATION: CD22
<400> SEQUENCE: 96
Met His Leu Leu Gly Pro Trp Leu Leu Leu Leu Val Leu Glu Tyr Leu
1 5 10 15
Ala Phe Ser Asp Ser Ser Lys Trp Val Phe Glu His Pro Glu Thr Leu
                        20 25 30
Tyr Ala Trp Glu Gly Ala Cys Val Trp Ile Pro Cys Thr Tyr Arg Ala
                35 40 45
Leu Asp Gly Asp Leu Glu Ser Phe Ile Leu Phe His Asn Pro Glu Tyr
        50 55 60
Asn Lys Asn Thr Ser Lys Phe Asp Gly Thr Arg Leu Tyr Glu Ser Thr
65 70 75 80
Lys Asp Gly Lys Val Pro Ser Glu Gln Lys Arg Val Gln Phe Leu Gly
                                85 90 95
Asp Lys Asn Lys Asn Cys Thr Leu Ser Ile His Pro Val His Leu Asn
                        100 105 110
Asp Ser Gly Gln Leu Gly Leu Arg Met Glu Ser Lys Thr Glu Lys Trp
                115 120 125
Met Glu Arg Ile His Leu Asn Val Ser Glu Arg Pro Phe Pro Pro His
        130 135 140
Ile Gln Leu Pro Pro Glu Ile Gln Glu Ser Gln Glu Val Thr Leu Thr
145 150 155 160
Cys Leu Leu Asn Phe Ser Cys Tyr Gly Tyr Pro Ile Gln Leu Gln Trp
                                165 170 175
Leu Leu Glu Gly Val Pro Met Arg Gln Ala Ala Val Thr Ser Thr Ser
                        180 185 190
Leu Thr Ile Lys Ser Val Phe Thr Arg Ser Glu Leu Lys Phe Ser Pro
                195 200 205
Gln Trp Ser His His Gly Lys Ile Val Thr Cys Gln Leu Gln Asp Ala
        210 215 220
Asp Gly Lys Phe Leu Ser Asn Asp Thr Val Gln Leu Asn Val Lys His
225 230 235 240
Pro Pro Lys Lys Val Thr Thr Val Ile Gln Asn Pro Met Pro Ile Arg
                                245 250 255
Glu Gly Asp Thr Val Thr Leu Ser Cys Asn Tyr Asn Ser Ser Asn Pro
                        260 265 270
Ser Val Thr Arg Tyr Glu Trp Lys Pro His Gly Ala Trp Glu Glu Pro
                275 280 285
Ser Leu Gly Val Leu Lys Ile Gln Asn Val Gly Trp Asp Asn Thr Thr
        290 295 300
Ile Ala Cys Ala Ala Cys Asn Ser Trp Cys Ser Trp Ala Ser Pro Val
305 310 315 320
Ala Leu Asn Val Gln Tyr Ala Pro Arg Asp Val Arg Val Arg Lys Ile
                                325 330 335
Lys Pro Leu Ser Glu Ile His Ser Gly Asn Ser Val Ser Leu Gln Cys
                        340 345 350
Asp Phe Ser Ser Ser His Pro Lys Glu Val Gln Phe Phe Trp Glu Lys
                355 360 365
Asn Gly Arg Leu Leu Gly Lys Glu Ser Gln Leu Asn Phe Asp Ser Ile
        370 375 380
Ser Pro Glu Asp Ala Gly Ser Tyr Ser Cys Trp Val Asn Asn Ser Ile
385 390 395 400
Gly Gln Thr Ala Ser Lys Ala Trp Thr Leu Glu Val Leu Tyr Ala Pro
                                405 410 415
Arg Arg Leu Arg Val Ser Met Ser Pro Gly Asp Gln Val Met Glu Gly
                        420 425 430
Lys Ser Ala Thr Leu Thr Cys Glu Ser Asp Ala Asn Pro Pro Val Ser
                435 440 445
His Tyr Thr Trp Phe Asp Trp Asn Asn Gln Ser Leu Pro Tyr His Ser
        450 455 460
Gln Lys Leu Arg Leu Glu Pro Val Lys Val Gln His Ser Gly Ala Tyr
465 470 475 480
<210> SEQ ID NO 97
<211> LENGTH: 321
<212> TYPE: PRT
<213> ORGANISM: Homo sapiens
<220> FEATURE:
<223> OTHER INFORMATION: CD23
<400> SEQUENCE: 97
Met Glu Glu Gly Gln Tyr Ser Glu Ile Glu Glu Leu Pro Arg Arg Arg
1 5 10 15
Cys Cys Arg Arg Gly Thr Gln Ile Val Leu Leu Gly Leu Val Thr Ala
                        20 25 30
Ala Leu Trp Ala Gly Leu Leu Thr Leu Leu Leu Leu Trp His Trp Asp
                35 40 45
Thr Thr Gln Ser Leu Lys Gln Leu Glu Glu Arg Ala Ala Arg Asn Val
        50 55 60
Ser Gln Val Ser Lys Asn Leu Glu Ser His His Gly Asp Gln Met Ala
65 70 75 80
Gln Lys Ser Gln Ser Thr Gln Ile Ser Gln Glu Leu Glu Glu Leu Arg
                                85 90 95
Ala Glu Gln Gln Arg Leu Lys Ser Gln Asp Leu Glu Leu Ser Trp Asn
                        100 105 110
Leu Asn Gly Leu Gln Ala Asp Leu Ser Ser Phe Lys Ser Gln Glu Leu
                115 120 125
Asn Glu Arg Asn Glu Ala Ser Asp Leu Leu Glu Arg Leu Arg Glu Glu
        130 135 140
Val Thr Lys Leu Arg Met Glu Leu Gln Val Ser Ser Gly Phe Val Cys
145 150 155 160
Asn Thr Cys Pro Glu Lys Trp Ile Asn Phe Gln Arg Lys Cys Tyr Tyr
                                165 170 175
Phe Gly Lys Gly Thr Lys Gln Trp Val His Ala Arg Tyr Ala Cys Asp
                        180 185 190
Asp Met Glu Gly Gln Leu Val Ser Ile His Ser Pro Glu Glu Gln Asp
                195 200 205
Phe Leu Thr Lys His Ala Ser His Thr Gly Ser Trp Ile Gly Leu Arg
        210 215 220
Asn Leu Asp Leu Lys Gly Glu Phe Ile Trp Val Asp Gly Ser His Val
225 230 235 240
Asp Tyr Ser Asn Trp Ala Pro Gly Glu Pro Thr Ser Arg Ser Gln Gly
                                245 250 255
Glu Asp Cys Val Met Met Arg Gly Ser Gly Arg Trp Asn Asp Ala Phe
                        260 265 270
Cys Asp Arg Lys Leu Gly Ala Trp Val Cys Asp Arg Leu Ala Thr Cys
                275 280 285
Thr Pro Pro Ala Ser Glu Gly Ser Ala Glu Ser Met Gly Pro Asp Ser
        290 295 300
Arg Pro Asp Pro Asp Gly Arg Leu Pro Thr Pro Ser Ala Pro Leu His
305 310 315 320
Ser
<210> SEQ ID NO 98
<211> LENGTH: 595
<212> TYPE: PRT
<213> ORGANISM: Homo sapiens
<220> FEATURE:
<223> OTHER INFORMATION: CD30
<400> SEQUENCE: 98
Met Arg Val Leu Leu Ala Ala Leu Gly Leu Leu Phe Leu Gly Ala Leu
1 5 10 15
Arg Ala Phe Pro Gln Asp Arg Pro Phe Glu Asp Thr Cys His Gly Asn
                        20 25 30
Pro Ser His Tyr Tyr Asp Lys Ala Val Arg Arg Cys Cys Tyr Arg Cys
                35 40 45
Pro Met Gly Leu Phe Pro Thr Gln Gln Cys Pro Gln Arg Pro Thr Asp
        50 55 60
Cys Arg Lys Gln Cys Glu Pro Asp Tyr Tyr Leu Asp Glu Ala Asp Arg
65 70 75 80
Cys Thr Ala Cys Val Thr Cys Ser Arg Asp Asp Leu Val Glu Lys Thr
                                85 90 95
Pro Cys Ala Trp Asn Ser Ser Arg Val Cys Glu Cys Arg Pro Gly Met
                        100 105 110
Phe Cys Ser Thr Ser Ala Val Asn Ser Cys Ala Arg Cys Phe Phe His
                115 120 125
Ser Val Cys Pro Ala Gly Met Ile Val Lys Phe Pro Gly Thr Ala Gln
        130 135 140
Lys Asn Thr Val Cys Glu Pro Ala Ser Pro Gly Val Ser Pro Ala Cys
145 150 155 160
Ala Ser Pro Glu Asn Cys Lys Glu Pro Ser Ser Gly Thr Ile Pro Gln
                                165 170 175
Ala Lys Pro Thr Pro Val Ser Pro Ala Thr Ser Ser Ala Ser Thr Met
                        180 185 190
Pro Val Arg Gly Gly Thr Arg Leu Ala Gln Glu Ala Ala Ser Lys Leu
                195 200 205
Thr Arg Ala Pro Asp Ser Pro Ser Ser Val Gly Arg Pro Ser Ser Asp
        210 215 220
Pro Gly Leu Ser Pro Thr Gln Pro Cys Pro Glu Gly Ser Gly Asp Cys
225 230 235 240
Arg Lys Gln Cys Glu Pro Asp Tyr Tyr Leu Asp Glu Ala Gly Arg Cys
                                245 250 255
Thr Ala Cys Val Ser Cys Ser Arg Asp Asp Leu Val Glu Lys Thr Pro
                        260 265 270
Cys Ala Trp Asn Ser Ser Arg Thr Cys Glu Cys Arg Pro Gly Met Ile
                275 280 285
Cys Ala Thr Ser Ala Thr Asn Ser Cys Ala Arg Cys Val Pro Tyr Pro
        290 295 300
Ile Cys Ala Ala Glu Thr Val Thr Lys Pro Gln Asp Met Ala Glu Lys
305 310 315 320
Asp Thr Thr Phe Glu Ala Pro Pro Leu Gly Thr Gln Pro Asp Cys Asn
                                325 330 335
Pro Thr Pro Glu Asn Gly Glu Ala Pro Ala Ser Thr Ser Pro Thr Gln
                        340 345 350
Ser Leu Leu Val Asp Ser Gln Ala Ser Lys Thr Leu Pro Ile Pro Thr
                355 360 365
Ser Ala Pro Val Ala Leu Ser Ser Thr Gly Lys Pro Val Leu Asp Ala
        370 375 380
Gly Pro Val Leu Phe Trp Val Ile Leu Val Leu Val Val Val Val Gly
385 390 395 400
Ser Ser Ala Phe Leu Leu Cys His Arg Arg Ala Cys Arg Lys Arg Ile
                                405 410 415
Arg Gln Lys Leu His Leu Cys Tyr Pro Val Gln Thr Ser Gln Pro Lys
                        420 425 430
Leu Glu Leu Val Asp Ser Arg Pro Arg Arg Ser Ser Thr Gln Leu Arg
                435 440 445
Ser Gly Ala Ser Val Thr Glu Pro Val Ala Glu Glu Arg Gly Leu Met
        450 455 460
Ser Gln Pro Leu Met Glu Thr Cys His Ser Val Gly Ala Ala Tyr Leu
465 470 475 480
Glu Ser Leu Pro Leu Gln Asp Ala Ser Pro Ala Gly Gly Pro Ser Ser
                                485 490 495
Pro Arg Asp Leu Pro Glu Pro Arg Val Ser Thr Glu His Thr Asn Asn
                        500 505 510
Lys Ile Glu Lys Ile Tyr Ile Met Lys Ala Asp Thr Val Ile Val Gly
                515 520 525
Thr Val Lys Ala Glu Leu Pro Glu Gly Arg Gly Leu Ala Gly Pro Ala
        530 535 540
Glu Pro Glu Leu Glu Glu Glu Leu Glu Ala Asp His Thr Pro His Tyr
545 550 555 560
Pro Glu Gln Glu Thr Glu Pro Pro Leu Gly Ser Cys Ser Asp Val Met
                                565 570 575
Leu Ser Val Glu Glu Glu Gly Lys Glu Asp Pro Leu Pro Thr Ala Ala
                        580 585 590
Ser Gly Lys
                595
<210> SEQ ID NO 99
<211> LENGTH: 400
<212> TYPE: PRT
<213> ORGANISM: Homo sapiens
<220> FEATURE:
<223> OTHER INFORMATION: CD43
<400> SEQUENCE: 99
Met Ala Thr Leu Leu Leu Leu Leu Gly Val Leu Val Val Ser Pro Asp
1 5 10 15
Ala Leu Gly Ser Thr Thr Ala Val Gln Thr Pro Thr Ser Gly Glu Pro
                        20 25 30
Leu Val Ser Thr Ser Glu Pro Leu Ser Ser Lys Met Tyr Thr Thr Ser
                35 40 45
Ile Thr Ser Asp Pro Lys Ala Asp Ser Thr Gly Asp Gln Thr Ser Ala
        50 55 60
Leu Pro Pro Ser Thr Ser Ile Asn Glu Gly Ser Pro Leu Trp Thr Ser
65 70 75 80
Ile Gly Ala Ser Thr Gly Ser Pro Leu Pro Glu Pro Thr Thr Tyr Gln
                                85 90 95
Glu Val Ser Ile Lys Met Ser Ser Val Pro Gln Glu Thr Pro His Ala
                        100 105 110
Thr Ser His Pro Ala Val Pro Ile Thr Ala Asn Ser Leu Gly Ser His
                115 120 125
Thr Val Thr Gly Gly Thr Ile Thr Thr Asn Ser Pro Glu Thr Ser Ser
        130 135 140
Arg Thr Ser Gly Ala Pro Val Thr Thr Ala Ala Ser Ser Leu Glu Thr
145 150 155 160
Ser Arg Gly Thr Ser Gly Pro Pro Leu Thr Met Ala Thr Val Ser Leu
                                165 170 175
Glu Thr Ser Lys Gly Thr Ser Gly Pro Pro Val Thr Met Ala Thr Asp
                        180 185 190
Ser Leu Glu Thr Ser Thr Gly Thr Thr Gly Pro Pro Val Thr Met Thr
                195 200 205
Thr Gly Ser Leu Glu Pro Ser Ser Gly Ala Ser Gly Pro Gln Val Ser
        210 215 220
Ser Val Lys Leu Ser Thr Met Met Ser Pro Thr Thr Ser Thr Asn Ala
225 230 235 240
Ser Thr Val Pro Phe Arg Asn Pro Asp Glu Asn Ser Arg Gly Met Leu
                                245 250 255
Pro Val Ala Val Leu Val Ala Leu Leu Ala Val Ile Val Leu Val Ala
                        260 265 270
Leu Leu Leu Leu Trp Arg Arg Arg Gln Lys Arg Arg Thr Gly Ala Leu
                275 280 285
Val Leu Ser Arg Gly Gly Lys Arg Asn Gly Val Val Asp Ala Trp Ala
        290 295 300
Gly Pro Ala Gln Val Pro Glu Glu Gly Ala Val Thr Val Thr Val Gly
305 310 315 320
Gly Ser Gly Gly Asp Lys Gly Ser Gly Phe Pro Asp Gly Glu Gly Ser
                                325 330 335
Ser Arg Arg Pro Thr Leu Thr Thr Phe Phe Gly Arg Arg Lys Ser Arg
                        340 345 350
Gln Gly Ser Leu Ala Met Glu Glu Leu Lys Ser Gly Ser Gly Pro Ser
                355 360 365
Leu Lys Gly Glu Glu Glu Pro Leu Val Ala Ser Glu Asp Gly Ala Val
        370 375 380
Asp Ala Pro Ala Pro Asp Glu Pro Glu Gly Gly Asp Gly Ala Ala Pro
385 390 395 400
<210> SEQ ID NO 100
<211> LENGTH: 742
<212> TYPE: PRT
<213> ORGANISM: Homo sapiens
<220> FEATURE:
<223> OTHER INFORMATION: CD44
<400> SEQUENCE: 100
Met Asp Lys Phe Trp Trp His Ala Ala Trp Gly Leu Cys Leu Val Pro
1 5 10 15
Leu Ser Leu Ala Gln Ile Asp Leu Asn Ile Thr Cys Arg Phe Ala Gly
                        20 25 30
Val Phe His Val Glu Lys Asn Gly Arg Tyr Ser Ile Ser Arg Thr Glu
                35 40 45
Ala Ala Asp Leu Cys Lys Ala Phe Asn Ser Thr Leu Pro Thr Met Ala
        50 55 60
Gln Met Glu Lys Ala Leu Ser Ile Gly Phe Glu Thr Cys Arg Tyr Gly
65 70 75 80
Phe Ile Glu Gly His Val Val Ile Pro Arg Ile His Pro Asn Ser Ile
                                85 90 95
Cys Ala Ala Asn Asn Thr Gly Val Tyr Ile Leu Thr Ser Asn Thr Ser
                        100 105 110
Gln Tyr Asp Thr Tyr Cys Phe Asn Ala Ser Ala Pro Pro Glu Glu Asp
                115 120 125
Cys Thr Ser Val Thr Asp Leu Pro Asn Ala Phe Asp Gly Pro Ile Thr
        130 135 140
Ile Thr Ile Val Asn Arg Asp Gly Thr Arg Tyr Val Gln Lys Gly Glu
145 150 155 160
Tyr Arg Thr Asn Pro Glu Asp Ile Tyr Pro Ser Asn Pro Thr Asp Asp
                                165 170 175
Asp Val Ser Ser Gly Ser Ser Ser Glu Arg Ser Ser Thr Ser Gly Gly
                        180 185 190
Tyr Ile Phe Tyr Thr Phe Ser Thr Val His Pro Ile Pro Asp Glu Asp
                195 200 205
Ser Pro Trp Ile Thr Asp Ser Thr Asp Arg Ile Pro Ala Thr Thr Leu
        210 215 220
Met Ser Thr Ser Ala Thr Ala Thr Glu Thr Ala Thr Lys Arg Gln Glu
225 230 235 240
Thr Trp Asp Trp Phe Ser Trp Leu Phe Leu Pro Ser Glu Ser Lys Asn
                                245 250 255
His Leu His Thr Thr Thr Gln Met Ala Gly Thr Ser Ser Asn Thr Ile
                        260 265 270
Ser Ala Gly Trp Glu Pro Asn Glu Glu Asn Glu Asp Glu Arg Asp Arg
                275 280 285
His Leu Ser Phe Ser Gly Ser Gly Ile Asp Asp Asp Glu Asp Phe Ile
        290 295 300
Ser Ser Thr Ile Ser Thr Thr Pro Arg Ala Phe Asp His Thr Lys Gln
305 310 315 320
Asn Gln Asp Trp Thr Gln Trp Asn Pro Ser His Ser Asn Pro Glu Val
                                325 330 335
Leu Leu Gln Thr Thr Thr Arg Met Thr Asp Val Asp Arg Asn Gly Thr
                        340 345 350
Thr Ala Tyr Glu Gly Asn Trp Asn Pro Glu Ala His Pro Pro Leu Ile
                355 360 365
His His Glu His His Glu Glu Glu Glu Thr Pro His Ser Thr Ser Thr
        370 375 380
Ile Gln Ala Thr Pro Ser Ser Thr Thr Glu Glu Thr Ala Thr Gln Lys
385 390 395 400
Glu Gln Trp Phe Gly Asn Arg Trp His Glu Gly Tyr Arg Gln Thr Pro
                                405 410 415
Lys Glu Asp Ser His Ser Thr Thr Gly Thr Ala Ala Ala Ser Ala His
                        420 425 430
Thr Ser His Pro Met Gln Gly Arg Thr Thr Pro Ser Pro Glu Asp Ser
                435 440 445
Ser Trp Thr Asp Phe Phe Asn Pro Ile Ser His Pro Met Gly Arg Gly
        450 455 460
His Gln Ala Gly Arg Arg Met Asp Met Asp Ser Ser His Ser Ile Thr
465 470 475 480
Leu Gln Pro Thr Ala Asn Pro Asn Thr Gly Leu Val Glu Asp Leu Asp
                                485 490 495
Arg Thr Gly Pro Leu Ser Met Thr Thr Gln Gln Ser Asn Ser Gln Ser
                        500 505 510
Phe Ser Thr Ser His Glu Gly Leu Glu Glu Asp Lys Asp His Pro Thr
                515 520 525
Thr Ser Thr Leu Thr Ser Ser Asn Arg Asn Asp Val Thr Gly Gly Arg
        530 535 540
Arg Asp Pro Asn His Ser Glu Gly Ser Thr Thr Leu Leu Glu Gly Tyr
545 550 555 560
Thr Ser His Tyr Pro His Thr Lys Glu Ser Arg Thr Phe Ile Pro Val
                                565 570 575
Thr Ser Ala Lys Thr Gly Ser Phe Gly Val Thr Ala Val Thr Val Gly
                        580 585 590
Asp Ser Asn Ser Asn Val Asn Arg Ser Leu Ser Gly Asp Gln Asp Thr
                595 600 605
Phe His Pro Ser Gly Gly Ser His Thr Thr His Gly Ser Glu Ser Asp
        610 615 620
Gly His Ser His Gly Ser Gln Glu Gly Gly Ala Asn Thr Thr Ser Gly
625 630 635 640
Pro Ile Arg Thr Pro Gln Ile Pro Glu Trp Leu Ile Ile Leu Ala Ser
                                645 650 655
Leu Leu Ala Leu Ala Leu Ile Leu Ala Val Cys Ile Ala Val Asn Ser
                        660 665 670
Arg Arg Arg Cys Gly Gln Lys Lys Lys Leu Val Ile Asn Ser Gly Asn
                675 680 685
Gly Ala Val Glu Asp Arg Lys Pro Ser Gly Leu Asn Gly Glu Ala Ser
        690 695 700
Lys Ser Gln Glu Met Val His Leu Val Asn Lys Glu Ser Ser Glu Thr
705 710 715 720
Pro Asp Gln Phe Met Thr Ala Asp Glu Thr Arg Asn Leu Gln Asn Val
                                725 730 735
Asp Met Lys Ile Gly Val
                        740
<210> SEQ ID NO 101
<211> LENGTH: 323
<212> TYPE: PRT
<213> ORGANISM: Homo sapiens
<220> FEATURE:
<223> OTHER INFORMATION: CD47
<400> SEQUENCE: 101
Met Trp Pro Leu Val Ala Ala Leu Leu Leu Gly Ser Ala Cys Cys Gly
1 5 10 15
Ser Ala Gln Leu Leu Phe Asn Lys Thr Lys Ser Val Glu Phe Thr Phe
                        20 25 30
Cys Asn Asp Thr Val Val Ile Pro Cys Phe Val Thr Asn Met Glu Ala
                35 40 45
Gln Asn Thr Thr Glu Val Tyr Val Lys Trp Lys Phe Lys Gly Arg Asp
        50 55 60
Ile Tyr Thr Phe Asp Gly Ala Leu Asn Lys Ser Thr Val Pro Thr Asp
65 70 75 80
Phe Ser Ser Ala Lys Ile Glu Val Ser Gln Leu Leu Lys Gly Asp Ala
                                85 90 95
Ser Leu Lys Met Asp Lys Ser Asp Ala Val Ser His Thr Gly Asn Tyr
                        100 105 110
Thr Cys Glu Val Thr Glu Leu Thr Arg Glu Gly Glu Thr Ile Ile Glu
                115 120 125
Leu Lys Tyr Arg Val Val Ser Trp Phe Ser Pro Asn Glu Asn Ile Leu
        130 135 140
Ile Val Ile Phe Pro Ile Phe Ala Ile Leu Leu Phe Trp Gly Gln Phe
145 150 155 160
Gly Ile Lys Thr Leu Lys Tyr Arg Ser Gly Gly Met Asp Glu Lys Thr
                                165 170 175
Ile Ala Leu Leu Val Ala Gly Leu Val Ile Thr Val Ile Val Ile Val
                        180 185 190
Gly Ala Ile Leu Phe Val Pro Gly Glu Tyr Ser Leu Lys Asn Ala Thr
                195 200 205
Gly Leu Gly Leu Ile Val Thr Ser Thr Gly Ile Leu Ile Leu Leu His
        210 215 220
Tyr Tyr Val Phe Ser Thr Ala Ile Gly Leu Thr Ser Phe Val Ile Ala
225 230 235 240
Ile Leu Val Ile Gln Val Ile Ala Tyr Ile Leu Ala Val Val Gly Leu
                                245 250 255
Ser Leu Cys Ile Ala Ala Cys Ile Pro Met His Gly Pro Leu Leu Ile
                        260 265 270
Ser Gly Leu Ser Ile Leu Ala Leu Ala Gln Leu Leu Gly Leu Val Tyr
                275 280 285
Met Lys Phe Val Ala Ser Asn Gln Lys Thr Ile Gln Pro Pro Arg Lys
        290 295 300
Ala Val Glu Glu Pro Leu Asn Ala Phe Lys Glu Ser Lys Gly Met Met
305 310 315 320
Asn Asp Glu
<210> SEQ ID NO 102
<211> LENGTH: 532
<212> TYPE: PRT
<213> ORGANISM: Homo sapiens
<220> FEATURE:
<223> OTHER INFORMATION: CD54
<400> SEQUENCE: 102
Met Ala Pro Ser Ser Pro Arg Pro Ala Leu Pro Ala Leu Leu Val Leu
1 5 10 15
Leu Gly Ala Leu Phe Pro Gly Pro Gly Asn Ala Gln Thr Ser Val Ser
                        20 25 30
Pro Ser Lys Val Ile Leu Pro Arg Gly Gly Ser Val Leu Val Thr Cys
                35 40 45
Ser Thr Ser Cys Asp Gln Pro Lys Leu Leu Gly Ile Glu Thr Pro Leu
        50 55 60
Pro Lys Lys Glu Leu Leu Leu Pro Gly Asn Asn Arg Lys Val Tyr Glu
65 70 75 80
Leu Ser Asn Val Gln Glu Asp Ser Gln Pro Met Cys Tyr Ser Asn Cys
                                85 90 95
Pro Asp Gly Gln Ser Thr Ala Lys Thr Phe Leu Thr Val Tyr Trp Thr
                        100 105 110
Pro Glu Arg Val Glu Leu Ala Pro Leu Pro Ser Trp Gln Pro Val Gly
                115 120 125
Lys Asn Leu Thr Leu Arg Cys Gln Val Glu Gly Gly Ala Pro Arg Ala
        130 135 140
Asn Leu Thr Val Val Leu Leu Arg Gly Glu Lys Glu Leu Lys Arg Glu
145 150 155 160
Pro Ala Val Gly Glu Pro Ala Glu Val Thr Thr Thr Val Leu Val Arg
                                165 170 175
Arg Asp His His Gly Ala Asn Phe Ser Cys Arg Thr Glu Leu Asp Leu
                        180 185 190
Arg Pro Gln Gly Leu Glu Leu Phe Glu Asn Thr Ser Ala Pro Tyr Gln
                195 200 205
Leu Gln Thr Phe Val Leu Pro Ala Thr Pro Pro Gln Leu Val Ser Pro
        210 215 220
Arg Val Leu Glu Val Asp Thr Gln Gly Thr Val Val Cys Ser Leu Asp
225 230 235 240
Gly Leu Phe Pro Val Ser Glu Ala Gln Val His Leu Ala Leu Gly Asp
                                245 250 255
Gln Arg Leu Asn Pro Thr Val Thr Tyr Gly Asn Asp Ser Phe Ser Ala
                        260 265 270
Lys Ala Ser Val Ser Val Thr Ala Glu Asp Glu Gly Thr Gln Arg Leu
                275 280 285
Thr Cys Ala Val Ile Leu Gly Asn Gln Ser Gln Glu Thr Leu Gln Thr
        290 295 300
Val Thr Ile Tyr Ser Phe Pro Ala Pro Asn Val Ile Leu Thr Lys Pro
305 310 315 320
Glu Val Ser Glu Gly Thr Glu Val Thr Val Lys Cys Glu Ala His Pro
                                325 330 335
Arg Ala Lys Val Thr Leu Asn Gly Val Pro Ala Gln Pro Leu Gly Pro
                        340 345 350
Arg Ala Gln Leu Leu Leu Lys Ala Thr Pro Glu Asp Asn Gly Arg Ser
                355 360 365
Phe Ser Cys Ser Ala Thr Leu Glu Val Ala Gly Gln Leu Ile His Lys
        370 375 380
Asn Gln Thr Arg Glu Leu Arg Val Leu Tyr Gly Pro Arg Leu Asp Glu
385 390 395 400
Arg Asp Cys Pro Gly Asn Trp Thr Trp Pro Glu Asn Ser Gln Gln Thr
                                405 410 415
Pro Met Cys Gln Ala Trp Gly Asn Pro Leu Pro Glu Leu Lys Cys Leu
                        420 425 430
Lys Asp Gly Thr Phe Pro Leu Pro Ile Gly Glu Ser Val Thr Val Thr
                435 440 445
Arg Asp Leu Glu Gly Thr Tyr Leu Cys Arg Ala Arg Ser Thr Gln Gly
        450 455 460
Glu Val Thr Arg Lys Val Thr Val Asn Val Leu Ser Pro Arg Tyr Glu
465 470 475 480
Ile Val Ile Ile Thr Val Val Ala Ala Ala Val Ile Met Gly Thr Ala
                                485 490 495
Gly Leu Ser Thr Tyr Leu Tyr Asn Arg Gln Arg Lys Ile Lys Lys Tyr
                        500 505 510
Arg Leu Gln Gln Ala Gln Lys Gly Thr Pro Met Lys Pro Asn Thr Gln
                515 520 525
Ala Thr Pro Pro
        530
<210> SEQ ID NO 103
<211> LENGTH: 381
<212> TYPE: PRT
<213> ORGANISM: Homo sapiens
<220> FEATURE:
<223> OTHER INFORMATION: CD55
<400> SEQUENCE: 103
Met Thr Val Ala Arg Pro Ser Val Pro Ala Ala Leu Pro Leu Leu Gly
1 5 10 15
Glu Leu Pro Arg Leu Leu Leu Leu Val Leu Leu Cys Leu Pro Ala Val
                        20 25 30
Trp Gly Asp Cys Gly Leu Pro Pro Asp Val Pro Asn Ala Gln Pro Ala
                35 40 45
Leu Glu Gly Arg Thr Ser Phe Pro Glu Asp Thr Val Ile Thr Tyr Lys
        50 55 60
Cys Glu Glu Ser Phe Val Lys Ile Pro Gly Glu Lys Asp Ser Val Ile
65 70 75 80
Cys Leu Lys Gly Ser Gln Trp Ser Asp Ile Glu Glu Phe Cys Asn Arg
                                85 90 95
Ser Cys Glu Val Pro Thr Arg Leu Asn Ser Ala Ser Leu Lys Gln Pro
                        100 105 110
Tyr Ile Thr Gln Asn Tyr Phe Pro Val Gly Thr Val Val Glu Tyr Glu
                115 120 125
Cys Arg Pro Gly Tyr Arg Arg Glu Pro Ser Leu Ser Pro Lys Leu Thr
        130 135 140
Cys Leu Gln Asn Leu Lys Trp Ser Thr Ala Val Glu Phe Cys Lys Lys
145 150 155 160
Lys Ser Cys Pro Asn Pro Gly Glu Ile Arg Asn Gly Gln Ile Asp Val
                                165 170 175
Pro Gly Gly Ile Leu Phe Gly Ala Thr Ile Ser Phe Ser Cys Asn Thr
                        180 185 190
Gly Tyr Lys Leu Phe Gly Ser Thr Ser Ser Phe Cys Leu Ile Ser Gly
                195 200 205
Ser Ser Val Gln Trp Ser Asp Pro Leu Pro Glu Cys Arg Glu Ile Tyr
        210 215 220
Cys Pro Ala Pro Pro Gln Ile Asp Asn Gly Ile Ile Gln Gly Glu Arg
225 230 235 240
Asp His Tyr Gly Tyr Arg Gln Ser Val Thr Tyr Ala Cys Asn Lys Gly
                                245 250 255
Phe Thr Met Ile Gly Glu His Ser Ile Tyr Cys Thr Val Asn Asn Asp
                        260 265 270
Glu Gly Glu Trp Ser Gly Pro Pro Pro Glu Cys Arg Gly Lys Ser Leu
                275 280 285
Thr Ser Lys Val Pro Pro Thr Val Gln Lys Pro Thr Thr Val Asn Val
        290 295 300
Pro Thr Thr Glu Val Ser Pro Thr Ser Gln Lys Thr Thr Thr Lys Thr
305 310 315 320
Thr Thr Pro Asn Ala Gln Ala Thr Arg Ser Thr Pro Val Ser Arg Thr
                                325 330 335
Thr Lys His Phe His Glu Thr Thr Pro Asn Lys Gly Ser Gly Thr Thr
                        340 345 350
Ser Gly Thr Thr Arg Leu Leu Ser Gly His Thr Cys Phe Thr Leu Thr
                355 360 365
Gly Leu Leu Gly Thr Leu Val Thr Met Gly Leu Leu Thr
        370 375 380
<210> SEQ ID NO 104
<211> LENGTH: 250
<212> TYPE: PRT
<213> ORGANISM: Homo sapiens
<220> FEATURE:
<223> OTHER INFORMATION: CD58
<400> SEQUENCE: 104
Met Val Ala Gly Ser Asp Ala Gly Arg Ala Leu Gly Val Leu Ser Val
1 5 10 15
Val Cys Leu Leu His Cys Phe Gly Phe Ile Ser Cys Phe Ser Gln Gln
                        20 25 30
Ile Tyr Gly Val Val Tyr Gly Asn Val Thr Phe His Val Pro Ser Asn
                35 40 45
Val Pro Leu Lys Glu Val Leu Trp Lys Lys Gln Lys Asp Lys Val Ala
        50 55 60
Glu Leu Glu Asn Ser Glu Phe Arg Ala Phe Ser Ser Phe Lys Asn Arg
65 70 75 80
Val Tyr Leu Asp Thr Val Ser Gly Ser Leu Thr Ile Tyr Asn Leu Thr
                                85 90 95
Ser Ser Asp Glu Asp Glu Tyr Glu Met Glu Ser Pro Asn Ile Thr Asp
                        100 105 110
Thr Met Lys Phe Phe Leu Tyr Val Leu Glu Ser Leu Pro Ser Pro Thr
                115 120 125
Leu Thr Cys Ala Leu Thr Asn Gly Ser Ile Glu Val Gln Cys Met Ile
        130 135 140
Pro Glu His Tyr Asn Ser His Arg Gly Leu Ile Met Tyr Ser Trp Asp
145 150 155 160
Cys Pro Met Glu Gln Cys Lys Arg Asn Ser Thr Ser Ile Tyr Phe Lys
                                165 170 175
Met Glu Asn Asp Leu Pro Gln Lys Ile Gln Cys Thr Leu Ser Asn Pro
                        180 185 190
Leu Phe Asn Thr Thr Ser Ser Ile Ile Leu Thr Thr Cys Ile Pro Ser
                195 200 205
Ser Gly His Ser Arg His Arg Tyr Ala Leu Ile Pro Ile Pro Leu Ala
        210 215 220
Val Ile Thr Thr Cys Ile Val Leu Tyr Met Asn Gly Ile Leu Lys Cys
225 230 235 240
Asp Arg Lys Pro Asp Arg Thr Asn Ser Asn
                                245 250
<210> SEQ ID NO 105
<211> LENGTH: 128
<212> TYPE: PRT
<213> ORGANISM: Homo sapiens
<220> FEATURE:
<223> OTHER INFORMATION: CD59
<400> SEQUENCE: 105
Met Gly Ile Gln Gly Gly Ser Val Leu Phe Gly Leu Leu Leu Val Leu
1 5 10 15
Ala Val Phe Cys His Ser Gly His Ser Leu Gln Cys Tyr Asn Cys Pro
                        20 25 30
Asn Pro Thr Ala Asp Cys Lys Thr Ala Val Asn Cys Ser Ser Asp Phe
                35 40 45
Asp Ala Cys Leu Ile Thr Lys Ala Gly Leu Gln Val Tyr Asn Lys Cys
        50 55 60
Trp Lys Phe Glu His Cys Asn Phe Asn Asp Val Thr Thr Arg Leu Arg
65 70 75 80
Glu Asn Glu Leu Thr Tyr Tyr Cys Cys Lys Lys Asp Leu Cys Asn Phe
                                85 90 95
Asn Glu Gln Leu Glu Asn Gly Gly Thr Ser Leu Ser Glu Lys Thr Val
                        100 105 110
Leu Leu Leu Val Thr Pro Phe Leu Ala Ala Ala Trp Ser Leu His Pro
                115 120 125
<210> SEQ ID NO 106
<211> LENGTH: 372
<212> TYPE: PRT
<213> ORGANISM: Homo sapiens
<220> FEATURE:
<223> OTHER INFORMATION: CD62L
<400> SEQUENCE: 106
Met Ile Phe Pro Trp Lys Cys Gln Ser Thr Gln Arg Asp Leu Trp Asn
1 5 10 15
Ile Phe Lys Leu Trp Gly Trp Thr Met Leu Cys Cys Asp Phe Leu Ala
                        20 25 30
His His Gly Thr Asp Cys Trp Thr Tyr His Tyr Ser Glu Lys Pro Met
                35 40 45
Asn Trp Gln Arg Ala Arg Arg Phe Cys Arg Asp Asn Tyr Thr Asp Leu
        50 55 60
Val Ala Ile Gln Asn Lys Ala Glu Ile Glu Tyr Leu Glu Lys Thr Leu
65 70 75 80
Pro Phe Ser Arg Ser Tyr Tyr Trp Ile Gly Ile Arg Lys Ile Gly Gly
                                85 90 95
Ile Trp Thr Trp Val Gly Thr Asn Lys Ser Leu Thr Glu Glu Ala Glu
                        100 105 110
Asn Trp Gly Asp Gly Glu Pro Asn Asn Lys Lys Asn Lys Glu Asp Cys
                115 120 125
Val Glu Ile Tyr Ile Lys Arg Asn Lys Asp Ala Gly Lys Trp Asn Asp
        130 135 140
Asp Ala Cys His Lys Leu Lys Ala Ala Leu Cys Tyr Thr Ala Ser Cys
145 150 155 160
Gln Pro Trp Ser Cys Ser Gly His Gly Glu Cys Val Glu Ile Ile Asn
                                165 170 175
Asn Tyr Thr Cys Asn Cys Asp Val Gly Tyr Tyr Gly Pro Gln Cys Gln
                        180 185 190
Phe Val Ile Gln Cys Glu Pro Leu Glu Ala Pro Glu Leu Gly Thr Met
                195 200 205
Asp Cys Thr His Pro Leu Gly Asn Phe Ser Phe Ser Ser Gln Cys Ala
        210 215 220
Phe Ser Cys Ser Glu Gly Thr Asn Leu Thr Gly Ile Glu Glu Thr Thr
225 230 235 240
Cys Gly Pro Phe Gly Asn Trp Ser Ser Pro Glu Pro Thr Cys Gln Val
                                245 250 255
Ile Gln Cys Glu Pro Leu Ser Ala Pro Asp Leu Gly Ile Met Asn Cys
                        260 265 270
Ser His Pro Leu Ala Ser Phe Ser Phe Thr Ser Ala Cys Thr Phe Ile
                275 280 285
Cys Ser Glu Gly Thr Glu Leu Ile Gly Lys Lys Lys Thr Ile Cys Glu
        290 295 300
Ser Ser Gly Ile Trp Ser Asn Pro Ser Pro Ile Cys Gln Lys Leu Asp
305 310 315 320
Lys Ser Phe Ser Met Ile Lys Glu Gly Asp Tyr Asn Pro Leu Phe Ile
                                325 330 335
Pro Val Ala Val Met Val Thr Ala Phe Ser Gly Leu Ala Phe Ile Ile
                        340 345 350
Trp Leu Ala Arg Arg Leu Lys Lys Gly Lys Lys Ser Lys Arg Ser Met
                355 360 365
Asn Asp Pro Tyr
        370
<210> SEQ ID NO 107
<211> LENGTH: 335
<212> TYPE: PRT
<213> ORGANISM: Homo sapiens
<220> FEATURE:
<223> OTHER INFORMATION: CD95
<400> SEQUENCE: 107
Met Leu Gly Ile Trp Thr Leu Leu Pro Leu Val Leu Thr Ser Val Ala
1 5 10 15
Arg Leu Ser Ser Lys Ser Val Asn Ala Gln Val Thr Asp Ile Asn Ser
                        20 25 30
Lys Gly Leu Glu Leu Arg Lys Thr Val Thr Thr Val Glu Thr Gln Asn
                35 40 45
Leu Glu Gly Leu His His Asp Gly Gln Phe Cys His Lys Pro Cys Pro
        50 55 60
Pro Gly Glu Arg Lys Ala Arg Asp Cys Thr Val Asn Gly Asp Glu Pro
65 70 75 80
Asp Cys Val Pro Cys Gln Glu Gly Lys Glu Tyr Thr Asp Lys Ala His
                                85 90 95
Phe Ser Ser Lys Cys Arg Arg Cys Arg Leu Cys Asp Glu Gly His Gly
                        100 105 110
Leu Glu Val Glu Ile Asn Cys Thr Arg Thr Gln Asn Thr Lys Cys Arg
                115 120 125
Cys Lys Pro Asn Phe Phe Cys Asn Ser Thr Val Cys Glu His Cys Asp
        130 135 140
Pro Cys Thr Lys Cys Glu His Gly Ile Ile Lys Glu Cys Thr Leu Thr
145 150 155 160
Ser Asn Thr Lys Cys Lys Glu Glu Gly Ser Arg Ser Asn Leu Gly Trp
                                165 170 175
Leu Cys Leu Leu Leu Leu Pro Ile Pro Leu Ile Val Trp Val Lys Arg
                        180 185 190
Lys Glu Val Gln Lys Thr Cys Arg Lys His Arg Lys Glu Asn Gln Gly
                195 200 205
Ser His Glu Ser Pro Thr Leu Asn Pro Glu Thr Val Ala Ile Asn Leu
        210 215 220
Ser Asp Val Asp Leu Ser Lys Tyr Ile Thr Thr Ile Ala Gly Val Met
225 230 235 240
Thr Leu Ser Gln Val Lys Gly Phe Val Arg Lys Asn Gly Val Asn Glu
                                245 250 255
Ala Lys Ile Asp Glu Ile Lys Asn Asp Asn Val Gln Asp Thr Ala Glu
                        260 265 270
Gln Lys Val Gln Leu Leu Arg Asn Trp His Gln Leu His Gly Lys Lys
                275 280 285
Glu Ala Tyr Asp Thr Leu Ile Lys Asp Leu Lys Lys Ala Asn Leu Cys
        290 295 300
Thr Leu Ala Glu Lys Ile Gln Thr Ile Ile Leu Lys Asp Ile Thr Ser
305 310 315 320
Asp Ser Glu Asn Ser Asn Phe Arg Asn Glu Ile Gln Ser Leu Val
                                325 330 335
<210> SEQ ID NO 108
<211> LENGTH: 13
<212> TYPE: PRT
<213> ORGANISM: Artificial sequence
<220> FEATURE:
<223> OTHER INFORMATION: T cell antigen
<400> SEQUENCE: 108
Pro Asp Asp Tyr Ser Asn Thr His Ser Thr Arg Tyr Val
1 5 10
<210> SEQ ID NO 109
<211> LENGTH: 11
<212> TYPE: PRT
<213> ORGANISM: Artificial Sequence
<220> FEATURE:
<223> OTHER INFORMATION: biotin labelled control peptide
<400> SEQUENCE: 109
Arg Pro His Glu Arg Asn Phe Gly Thr Val Leu
1 5 10
<210> SEQ ID NO 110
<211> LENGTH: 15
<212> TYPE: PRT
<213> ORGANISM: Artificial Sequence
<220> FEATURE:
<223> OTHER INFORMATION: PADRE MHC Class-II epitope
<220> FEATURE:
<221> NAME/KEY: SITE
<222> LOCATION: 1.13
<223> OTHER INFORMATION: wherein the alanine is d-alanine
<220> FEATURE:
<221> NAME/KEY: MISC_FEATURE
<222> LOCATION: (14)..(14)
<223> OTHER INFORMATION: X is Acp (6-Aminocaproic acid)
<400> SEQUENCE: 110
Ala Lys Ala Val Ala Ala Trp Thr Leu Lys Ala Ala Ala Xaa Cys
1 5 10 15
<210> SEQ ID NO 111
<211> LENGTH: 847
<212> TYPE: PRT
<213> ORGANISM: Homo sapiens
<220> FEATURE:
<223> OTHER INFORMATION: Beta form of CD22
<400> SEQUENCE: 111
Met His Leu Leu Gly Pro Trp Leu Leu Leu Leu Val Leu Glu Tyr Leu
1 5 10 15
Ala Phe Ser Asp Ser Ser Lys Trp Val Phe Glu His Pro Glu Thr Leu
                        20 25 30
Tyr Ala Trp Glu Gly Ala Cys Val Trp Ile Pro Cys Thr Tyr Arg Ala
                35 40 45
Leu Asp Gly Asp Leu Glu Ser Phe Ile Leu Phe His Asn Pro Glu Tyr
        50 55 60
Asn Lys Asn Thr Ser Lys Phe Asp Gly Thr Arg Leu Tyr Glu Ser Thr
65 70 75 80
Lys Asp Gly Lys Val Pro Ser Glu Gln Lys Arg Val Gln Phe Leu Gly
                                85 90 95
Asp Lys Asn Lys Asn Cys Thr Leu Ser Ile His Pro Val His Leu Asn
                        100 105 110
Asp Ser Gly Gln Leu Gly Leu Arg Met Glu Ser Lys Thr Glu Lys Trp
                115 120 125
Met Glu Arg Ile His Leu Asn Val Ser Glu Arg Pro Phe Pro Pro His
        130 135 140
Ile Gln Leu Pro Pro Glu Ile Gln Glu Ser Gln Glu Val Thr Leu Thr
145 150 155 160
Cys Leu Leu Asn Phe Ser Cys Tyr Gly Tyr Pro Ile Gln Leu Gln Trp
                                165 170 175
Leu Leu Glu Gly Val Pro Met Arg Gln Ala Ala Val Thr Ser Thr Ser
                        180 185 190
Leu Thr Ile Lys Ser Val Phe Thr Arg Ser Glu Leu Lys Phe Ser Pro
                195 200 205
Gln Trp Ser His His Gly Lys Ile Val Thr Cys Gln Leu Gln Asp Ala
        210 215 220
Asp Gly Lys Phe Leu Ser Asn Asp Thr Val Gln Leu Asn Val Lys His
225 230 235 240
Thr Pro Lys Leu Glu Ile Lys Val Thr Pro Ser Asp Ala Ile Val Arg
                                245 250 255
Glu Gly Asp Ser Val Thr Met Thr Cys Glu Val Ser Ser Ser Asn Pro
                        260 265 270
Glu Tyr Thr Thr Val Ser Trp Leu Lys Asp Gly Thr Ser Leu Lys Lys
                275 280 285
Gln Asn Thr Phe Thr Leu Asn Leu Arg Glu Val Thr Lys Asp Gln Ser
        290 295 300
Gly Lys Tyr Cys Cys Gln Val Ser Asn Asp Val Gly Pro Gly Arg Ser
305 310 315 320
Glu Glu Val Phe Leu Gln Val Gln Tyr Ala Pro Glu Pro Ser Thr Val
                                325 330 335
Gln Ile Leu His Ser Pro Ala Val Glu Gly Ser Gln Val Glu Phe Leu
                        340 345 350
Cys Met Ser Leu Ala Asn Pro Leu Pro Thr Asn Tyr Thr Trp Tyr His
                355 360 365
Asn Gly Lys Glu Met Gln Gly Arg Thr Glu Glu Lys Val His Ile Pro
        370 375 380
Lys Ile Leu Pro Trp His Ala Gly Thr Tyr Ser Cys Val Ala Glu Asn
385 390 395 400
Ile Leu Gly Thr Gly Gln Arg Gly Pro Gly Ala Glu Leu Asp Val Gln
                                405 410 415
Tyr Pro Pro Lys Lys Val Thr Thr Val Ile Gln Asn Pro Met Pro Ile
                        420 425 430
Arg Glu Gly Asp Thr Val Thr Leu Ser Cys Asn Tyr Asn Ser Ser Asn
                435 440 445
Pro Ser Val Thr Arg Tyr Glu Trp Lys Pro His Gly Ala Trp Glu Glu
        450 455 460
Pro Ser Leu Gly Val Leu Lys Ile Gln Asn Val Gly Trp Asp Asn Thr
465 470 475 480
Thr Ile Ala Cys Ala Ala Cys Asn Ser Trp Cys Ser Trp Ala Ser Pro
                                485 490 495
Val Ala Leu Asn Val Gln Tyr Ala Pro Arg Asp Val Arg Val Arg Lys
                        500 505 510
Ile Lys Pro Leu Ser Glu Ile His Ser Gly Asn Ser Val Ser Leu Gln
                515 520 525
Cys Asp Phe Ser Ser Ser His Pro Lys Glu Val Gln Phe Phe Trp Glu
        530 535 540
Lys Asn Gly Arg Leu Leu Gly Lys Glu Ser Gln Leu Asn Phe Asp Ser
545 550 555 560
Ile Ser Pro Glu Asp Ala Gly Ser Tyr Ser Cys Trp Val Asn Asn Ser
                                565 570 575
Ile Gly Gln Thr Ala Ser Lys Ala Trp Thr Leu Glu Val Leu Tyr Ala
                        580 585 590
Pro Arg Arg Leu Arg Val Ser Met Ser Pro Gly Asp Gln Val Met Glu
                595 600 605
Gly Lys Ser Ala Thr Leu Thr Cys Glu Ser Asp Ala Asn Pro Pro Val
        610 615 620
Ser His Tyr Thr Trp Phe Asp Trp Asn Asn Gln Ser Leu Pro Tyr His
625 630 635 640
Ser Gln Lys Leu Arg Leu Glu Pro Val Lys Val Gln His Ser Gly Ala
                                645 650 655
Tyr Trp Cys Gln Gly Thr Asn Ser Val Gly Lys Gly Arg Ser Pro Leu
                        660 665 670
Ser Thr Leu Thr Val Tyr Tyr Ser Pro Glu Thr Ile Gly Arg Arg Val
                675 680 685
Ala Val Gly Leu Gly Ser Cys Leu Ala Ile Leu Ile Leu Ala Ile Cys
        690 695 700
Gly Leu Lys Leu Gln Arg Arg Trp Lys Arg Thr Gln Ser Gln Gln Gly
705 710 715 720
Leu Gln Glu Asn Ser Ser Gly Gln Ser Phe Phe Val Arg Asn Lys Lys
                                725 730 735
Val Arg Arg Ala Pro Leu Ser Glu Gly Pro His Ser Leu Gly Cys Tyr
                        740 745 750
Asn Pro Met Met Glu Asp Gly Ile Ser Tyr Thr Thr Leu Arg Phe Pro
                755 760 765
Glu Met Asn Ile Pro Arg Thr Gly Asp Ala Glu Ser Ser Glu Met Gln
        770 775 780
Arg Pro Pro Pro Asp Cys Asp Asp Thr Val Thr Tyr Ser Ala Leu His
785 790 795 800
Lys Arg Gln Val Gly Asp Tyr Glu Asn Val Ile Pro Asp Phe Pro Glu
                                805 810 815
Asp Glu Gly Ile His Tyr Ser Glu Leu Ile Gln Phe Gly Val Gly Glu
                        820 825 830
Arg Pro Gln Ala Gln Glu Asn Val Asp Tyr Val Ile Leu Lys His
                835 840 845
<210> SEQ ID NO 112
<211> LENGTH: 237
<212> TYPE: PRT
<213> ORGANISM: Homo sapiens
<220> FEATURE:
<223> OTHER INFORMATION: Isoform 2 of CD58
<400> SEQUENCE: 112
Met Val Ala Gly Ser Asp Ala Gly Arg Ala Leu Gly Val Leu Ser Val
1 5 10 15
Val Cys Leu Leu His Cys Phe Gly Phe Ile Ser Cys Phe Ser Gln Gln
                        20 25 30
Ile Tyr Gly Val Val Tyr Gly Asn Val Thr Phe His Val Pro Ser Asn
                35 40 45
Val Pro Leu Lys Glu Val Leu Trp Lys Lys Gln Lys Asp Lys Val Ala
        50 55 60
Glu Leu Glu Asn Ser Glu Phe Arg Ala Phe Ser Ser Phe Lys Asn Arg
65 70 75 80
Val Tyr Leu Asp Thr Val Ser Gly Ser Leu Thr Ile Tyr Asn Leu Thr
                                85 90 95
Ser Ser Asp Glu Asp Glu Tyr Glu Met Glu Ser Pro Asn Ile Thr Asp
                        100 105 110
Thr Met Lys Phe Phe Leu Tyr Val Leu Glu Ser Leu Pro Ser Pro Thr
                115 120 125
Leu Thr Cys Ala Leu Thr Asn Gly Ser Ile Glu Val Gln Cys Met Ile
        130 135 140
Pro Glu His Tyr Asn Ser His Arg Gly Leu Ile Met Tyr Ser Trp Asp
145 150 155 160
Cys Pro Met Glu Gln Cys Lys Arg Asn Ser Thr Ser Ile Tyr Phe Lys
                                165 170 175
Met Glu Asn Asp Leu Pro Gln Lys Ile Gln Cys Thr Leu Ser Asn Pro
                        180 185 190
Leu Phe Asn Thr Thr Ser Ser Ile Ile Leu Thr Thr Cys Ile Pro Ser
                195 200 205
Ser Gly His Ser Arg His Arg Tyr Ala Leu Ile Pro Ile Pro Leu Ala
        210 215 220
Val Ile Thr Thr Cys Ile Val Leu Tyr Met Asn Val Leu
225 230 235
```

What is claimed is:

1. An agent comprising:
(i) a T cell antigen, and
(ii) a binding partner for any of CD22, CD23, CD30, CD74, CD70, CD43, CD44, CD47, CD54, CD58, CD62L, CD95, HLA-DR, CD59, CD55,
wherein, following binding of the agent to a cell that expresses any of CD22, CD23, CD30, CD74, CD70, CD43, CD44, CD47, CD54, CD58, CD62L, CD95, HLA-DR, CD59, CD55, the agent is internalised and the T cell antigen is presented on the surface of the cell in a form that can be recognised by a T cell; optionally the T cell antigen can be released intracellularly, optionally by an intracellular protease, within the cell that expresses any of CD22, CD23, CD30, CD74, CD70, CD43, CD44, CD47, CD54, CD58, CD62L, CD95, HLA-DR, CD59, CD55; optionally the agent is internalised and the T cell antigen is presented on the surface of the cell by binding to a MHC molecule or Group I CD1 molecule.

2. (canceled)

3. (canceled)

4. (canceled)

5. An agent according to claim 1 wherein the binding partner for any of CD22, CD23, CD30, CD74, CD70, CD43, CD44, CD47, CD54, CD58, CD62L, CD95, HLA-DR, CD59, CD55, is any of an antibody, a hormone, a growth factor, a cytokine, or a receptor ligand.

6. (canceled)

7. An agent according to claim 5, wherein the antibody is an anti-CD70 antibody.

8. An agent according to claim 5, wherein the antibody is an anti-CD74 antibody.

9. An agent according claim 1, wherein the T cell antigen is one that is capable of eliciting an existing T cell response in a subjects optionally the T cell antigen is any of a peptide, a polypeptide, a phosphopeptide or a lipid.

10. (canceled)

11. An agent according to claim 9, wherein the antigen is a viral-derived antigen; optionally the antigen is derived from any of Epstein Barr virus cytomegalovirus, Varicella Zoster virus Herpes simplex virus, adenovirus, rhinovirus, influenza virus, or derived from a vaccine such as tetanus toxoid.

12. (canceled)

13. An agent according to claim 1, wherein the T cell antigen is an MHC Class II restricted antigen, or an antigen that is capable of binding to a group I CD1 molecule.

14. (canceled)

15. A pharmaceutical composition, comprising an agent according to claim 1, and a pharmaceutically acceptable carrier, diluent or excipient.

16. A method of preventing or treating a condition characterised by the presence of cells expressing any of CD22, CD23, CD30, CD74, CD70, CD43, CD44, CD47, CD54, CD58, CD62L, CD95, HLA-DR, CD59, CD55, the method comprising administering an agent according to claim 1; optionally further comprising administering a further therapeutic agent to the subject.

17. (canceled)

18. (canceled)

19. A method according to claim 16, wherein before the step of administering the agent to the subject, one or both of (i) the MHC alleles of the subject, and (ii) the cytotoxic T cell response of the subject to a T cell antigen, is determined.

20. (canceled)

21. A composition or kit comprising (i) an agent according to claim 1 and (ii) a further therapeutic agent; optionally for use in preventing or treating a condition characterised by the presence of cells expressing any of CD22, CD23, CD30, CD74, CD70, CD43, CD44, CD47, CD54, CD58, CD62L, CD95, HLA-DR, CD59, CD55.

22. (canceled)

23. (canceled)

24. (canceled)

25. (canceled)

26. (canceled)

27. (canceled)

28. A method according to claim 16, wherein the condition characterised by the presence of cells expressing any of CD22, CD23, CD30, CD74, CD70, CD43, CD44, CD47, CD54, CD58, CD62L, CD95, HLA-DR, CD59, CD55, is any of a tumour (benign or malignant) or an autoimmune condition.

29. A method according to claim 16, wherein the condition characterised by the presence of cells expressing any of CD22, CD23, CD30, CD74, CD70, CD43, CD44, CD47, CD54, CD58, CD62L, CD95, HLA-DR, CD59, CD55, is a tumour, and the T cell antigen in the agent is a peptide.

30. (canceled)

31. A method according claim 16, wherein the further therapeutic agent is any one or more of a vaccine, an immunostimulatory drug, a live virus, an anti-cancer agent, an inhibitor of an antibody response against the agent of the invention, and a protease inhibitor.
